(19) United States
(12) Patent Application Publication
(10) Pub. No.: US 2025/0342907 A1
(43) Pub. Date: Nov. 6, 2025

(54) Identification of Fate-Determining Genes and Application of reconstructed Hematopoietic hierarchy

(71) Applicant: SHENZHEN PEOPLE’S HOSPITAL, Shenzhen, Guangdong (CN)

(72) Inventor: Yongjian YUE, Shenzhen (CN)

(21) Appl. No.: 19/273,122

(22) Filed: Jul. 17, 2025

Related U.S. Application Data
(63) Continuation of application No. PCT/CN2024/070855, filed on Jan. 5, 2024.

(30) Foreign Application Priority Data
Jan. 18, 2023 (CN) 202310055372.3
Jan. 19, 2023 (CN) 202310059162.1
Jan. 19, 2023 (CN) 202310060346.X
Dec. 8, 2023 (CN) 202311679968.7

(51) Int. Cl. G16B 20/00; G16B 45/00

(52) U.S. Cl. CPC G16B 20/00; G16B 45/00

(57) ABSTRACT

The identification of differentiation stages, differentiation trajectories, and expression profiles of hematopoietic progenitor cells and fate-determining factors, and the application thereof are provided. The enrichment and identification of rare hematopoietic progenitor cells, as well as the identification of and their fate-determining genes, are also provided. A method for reconstructing hematopoietic hierarchy is provided, which includes fate-determining factors, differentiation trajectories, and patterns within lineage commitment processes.

### CROSS-REFERENCE TO RELATED APPLICATIONS

The present application is a continuation of International Application No. PCT/CN2024/070855, with an international filing date of Jan. 1, 2024, which is based upon and claims priority to Chinese application numbers 202310055372.3, filed on Jan. 18, 2023; 202310059162.1, filed on Jan. 19, 2023; 202311679968.7, filed on Dec. 8, 2023; and 202310060346.X, filed on Jan. 19, 2023. The entire content of these applications is incorporated herein by reference.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: one 4,620 Byte XML file named “Sequence listing.xml,” dated Jul. 16, 2025.

### TECHNICAL FIELD

The present application relates to the field of biomedical technology, specifically to methods for enriching and identifying hematopoietic progenitor cells. These methods can identify hematopoietic progenitor cell lineages and characteristics at various differentiation stages and directions, and also identify the fate-determining genes and marker genes' characteristic expression profiles of progenitor subpopulations within each lineage, aiming to reconstruct the differentiation lineage tree of progenitor cells (hematopoietic hierarchy). By utilizing the progenitor cell lineage features or gene expression profile characteristics, in combination with the characteristics of hematopoietic hierarchy, the present application enables the uses in areas such as progenitor cell detection, isolation, differentiation control, induced culture, lineage tracking, reprogramming and the like.

### BACKGROUND

Identification of Hematopoietic Progenitor Cells Lineage and Definition and Fate-determining Factors

Various cells of human immune and blood systems originate and differentiate from hematopoietic stem cells (HSCs). During the differentiation and maturation process of HSCs (lineage commitment), they differentiate into precursor progenitor cells of various blood cell lineages, called hematopoietic progenitor cells (HPCs), and the lineage differentiation mainly includes myeloid progenitor cells and lymphoid progenitor cells. Human hematopoietic stem cells are scarce, CD34-positive hematopoietic stem cells are less than one in ten thousand cells in peripheral blood. Moreover, their subpopulations are complex and diverse, making it difficult to obtain enough cells for research, and it is challenging to clearly identify the differentiation stages, molecular characteristics, and differentiation pathways, so this has become a major obstacle and challenge for research and translational applications. The traditional definition and identification of hematopoietic stem cells are mainly based on surface markers such as CD34, CD90, CD45, CD135, CD117, etc. Functional studies are largely based on experimental methods like flow cytometry sorting of stem cells followed by in vivo transplantation and in vitro culture. However, both in vitro stem cell culture research and in vivo animal model experiments have significant limitations due to different experimental factors. Moreover, because the factors that influence the differentiation direction of myeloid and lymphoid lineages in hematopoietic stem cells are almost entirely different, studies typically focus on inducing a single lineage or its function at a time, making it difficult to accurately and comprehensively reflect the functions and differentiation states of the entire hematopoietic stem cell lineage. With the advancement of stem cell detection technologies and the emergence of single-cell sequencing, new definitions and classifications of HSCs have been proposed. It is known that HSC populations exhibit significant heterogeneity, with different progenitor cell subpopulations showing differentiation and functional heterogeneity. Therefore, the identification, classification, and function studies of HSCs have always been subjects of great debate. Investigation on hematopoietic stem cells continues to face persistent challenges since their initial discovery in 1961. In particular, the key factors determining the fate of hematopoietic stem cells and progenitor cells during maturation and differentiation, as well as the mechanisms behind these decisions, remain unclear.

In summary, due to the unclear and ambiguous of definitions, differentiation, fate determination and lineage commitment of hematopoietic stem and progenitor cells, it has not even been possible to effectively isolate and enrich specific rare subpopulations. This makes it difficult to precisely control the differentiation of individual progenitor cell types, especially with regard to accurately manipulating progenitor cells at various stages of lineage differentiation. The complexity of progenitor cell lineages and the unknown determine factors present key obstacles in the research and application of hematopoietic stem and progenitor cells for gene engineering, cell engineering, and culture induction.

2. The Fate Determining Transcription Factors of Hematopoietic Progenitor Cell Lineages and their Application Value

Elucidating the expression of HSCs and HPCs lineages, especially fate-determining factors governing lineage commitment enables cellular reprogramming, precise control of differentiation trajectories, and suppression of aberrant progenitor cell proliferation, can enable precise induction and transplantation of progenitor cell subpopulations, and apply to disease treatment.

Since 2006, when Shinya Yamanaka elucidated and established the method for inducing stem cell differentiation using the transcription factors Oct3/4, Sox2, c-Myc, and Klf4, induced pluripotent stem cell (iPSC) technology has become increasingly mature. For example, in 2014, Jonah Riddell et al. induced reprogramming to generate hematopoietic stem cells using six transcription factors. In 2016, Cedric Ghevaert et al. overexpressed the transcription factors GATA1, FLI1, and TAL1 in human pluripotent stem cells (hPSCs), generating a population of progenitor cells with directed differentiation potential into megakaryocytes and erythrocytes. In 2022, the NHS Blood and Transplant Center in UK, in collaboration with the Bristol University, the Cambridge University, and other institutions, initiated the world's first clinical trial of artificial red blood cells. Furthermore, by inhibiting key transcription factors, transcription factors can also be applied to disease treatment and intervention. For example, in 2013, R. Pattabiraman et al. found that the transcription factor MYB is a potential target for leukemia therapy; in 2021, Fanny Gonzales et al. discovered that inhibiting RUNX1 could control the progression of acute myeloid leukemia, providing novel therapeutic and application value. Therefore, these studies suggested that the intervening, transfecting, and activating key fate-determining factors of lineage cells, especially transcription factors, can be applied to induction of stem cells, reprogram, manipulation of progenitor cells and may be used to cancer treatment.

### 3. Hematopoietic Progenitor Cell Lineage Tree and the Hierarchy of Hematopoiesis

Regarding the immune and blood lineage tree, traditional differentiation models (hierarchy of hematopoiesis) suggest that HSCs initially differentiation into common lymphoid progenitor cells (CLP) and common myeloid progenitor cells (CMP), and CLPs further differentiate into T cells, B cells, and NK cells, while CMPs differentiate into erythrocytes, monocytes, and neutrophils. Based on in vitro functional and in vivo transplantation experiments, various hematopoietic hierarchy have been established. However, the current existing hierarchy of hematopoiesis (lineage tree) have below limitations:
It is unclear why certain subpopulations of myeloid stem cells can differentiate into multiple or other lineage cells;
It is still unclear whether megakaryocyte-erythroid progenitor cells arise from myeloid progenitors or multipotent progenitors, and their differentiation pathways remain undefined;
The intermediate stages of progenitor cell differentiation, as well as the upstream and downstream sources of various progenitor cell lineages, are highly disputed and have not reached a consensus;
It is still unclear which factors regulate and determine the appearance of differentiation branches during lineage commitment.

Clearly, the maturation and differentiation process of hematopoietic stem cell lineage (blood lineage) has the following characteristics:
Including different differentiation stages;
Differentiation branches progressively emerge, eventually forming different types of cells;
The fate-determining factors involved in different lineage branches are diversity, and key transcription factors determine the fate of progenitor cells and control their differentiation direction during lineage commitment.

In summary, the hierarchy of hematopoiesis remains controversial due to the unclear definition and unrevealed fate-determining factors of HPCs. Many fate-determining genes of hematopoietic stem cells have been identified, and methods for reprogramming and cell detection have been extensively reported. However, due to the lineage characteristics, gene characteristics, and fate-determining factors at each specific differentiation stage and pathway remain unclear, this presents a critical barrier to the application of hematopoietic progenitor cells. By elucidating the stages characteristic, pathways and directions, branch points, and lineage specific fate-determining factors of HPC differentiation, it can enable precise manipulating of HPC lineage commitment and hematopoiesis at different differentiation time, branch points and stages. This can also help inhibit excessive differentiation of stem cells and induce stem cell reprogramming. Ultimately, this will advance applications in the treatment of hematological and immunological diseases.

### SUMMARY

The main problems addressed by the present application are as follows:

To establish an efficient method for the enrichment and identification of rare hematopoietic stem cells and progenitor cells.

To accurately identify and redefine the progenitor cells of HPCs in peripheral blood, as well as their marker genes and fate-determining genes.

To reconstruct the hierarchy of hematopoiesis. The present breakthrough lies in solving the identification of progenitor cells, progenitor cell markers, key fate-determining genes of progenitor cells, and their differentiation stages (positions), hierarchy, and spatiotemporal characteristics during differentiation, which enables applications in various fields.

The application of marker genes, fate-determining genes, and hierarchical characteristics of hematopoietic progenitor cells in HSC detection, fate determination, induction culture, reprogramming, specific sorting, localization and lineage tracing. Ultimately, this enables spatiotemporally defined gene expression profiling and applications, cell type detection and application, as well as the spatiotemporal control and application of progenitor cells.

Methods for determining the fate-determining genes of HSCs, reprogramming, and cell detection have been widely reported; however, the key innovative point of the present application, distinguishing it from previous methods, is the integration of progenitor cell characteristics, expression profile features, and the hematopoietic hierarchy. By integrating these three features, the present invention enables novel applications, including progenitor cell detection, localization, reprogramming, fate control, treatment, and more.

In simple, based on the reconstructed hematopoietic hierarchy, combined with the cell lineage characteristics and gene expression profiles, this invention achieves precise spatiotemporal localization of each progenitor subpopulation's stage, differentiation pathway (differentiation trajectory), and direction, unlocking entirely new application value and scenarios. By controlling the switch genes (fate-determining genes) and pathways (branching, nodes, and positions within the hierarchy), the detection and control of progenitor cell differentiation stages, pathways (trajectory), and directions become possible. Because gene expression profiles exhibit distinct stage-specificity and encode information about a cell's differentiation stage and pathway (features previously unobtainable due to undefined pathways or an incorrect hierarchy), this invention, leveraging a precisely defined hematopoietic hierarchy and employing an innovative approach distinct from conventional cell detection methods, enables not only the determination of a cell population's type, quantity, and proportional composition, but also the accurate detection of the specific differentiation stage and trajectory of individual cells. By utilizing characteristic gene expression profiles combined with the hematopoietic hierarchy (serving as a navigational map for detection), it facilitates various applications for spatiotemporally defined cell detection, isolation, or enrichment.

### First Aspect

The present invention provided isolated hematopoietic progenitor cell populations.

The isolated hematopoietic progenitor cell populations provided by the present invention include the following subpopulations:
Common lymphoid progenitor subpopulation (CLPs),
NK progenitor subpopulation (Pro-NK),
T progenitor subpopulation (Pro-T),
B progenitor subpopulation (Pro-B),
Plasma progenitor subpopulation (Pro-Plasma),
Neutrophil and monocyte progenitor subpopulation (NMPs),
Megakaryocyte-erythroid lineage progenitor subpopulation (GAPs),
Megakaryocyte-erythroid progenitor subpopulation (MEPs),
Megakaryocyte-erythroid precursor progenitor subpopulation (Pro-ME),
Mast cell and basophil progenitor subpopulation (MBPs),
Eosinophil progenitor subpopulation (Pro-Eosinophil),
Monocyte-macrophage progenitor subpopulation (Pro-Mac),
Monocyte-dendritic cell progenitor subpopulation (Pro-DC).

Each subpopulation expresses specific genes and has distinct characteristics. For example:

The Common Lymphoid Progenitor subpopulation (CLPs) expresses genes such as SPINK2, HOPX, HOXA9, RUNX2, and others, while showing low or absent expression of CNRIP1, FCER1A, GATA1, and S100A10.

The NK Progenitor subpopulation (Pro-NK) expresses genes like GNLY, NKG7, CD247, CCL5, and others, with low or absent expression of IL7R and GATA3.

The T Progenitor subpopulation (Pro-T) expresses genes such as TCF7, IL7R, GATA3, KLRB1, and others, with low or absent expression of GNLY, FCGR3A, and GZMA.

The B Progenitor subpopulation (Pro-B) expresses genes such as CD19, MS4A1, FCER2, and others, with low or absent expression of CD27.

The Plasma Progenitor subpopulation (Pro-Plasma) expresses genes such as CD27, CD38, IGKC, IGHA1, and others, with absent expression of MS4A1 and FCER2.

Additionally, the Neutrophil and Monocyte Progenitor subpopulation (NMPs) expresses genes like CSF3R, MPO, MGST1, MYB, CDK4, and others, with low or absent expression of GATA2, SLC40A1, and others. The Megakaryocyte-Erythroid Lineage Progenitor subpopulation (GAPs) expresses genes like GATA2, NFE2, LYL1, MYB, and others, with absent expression of GATA1 and KLF1.

### Further Details on Specific Progenitor Populations:

The Megakaryocyte-Erythroid Progenitor subpopulation (MEPs) expresses genes such as GATA2, NFE2, LYL1, MYB, GATA1, KLF1, and others, with absent expression of CSF3R.

The Megakaryocyte-Erythroid Precursor Progenitor subpopulation (Pro-ME) expresses genes like HBD, MCM2, MCM6, and others, with low or absent expression of FLT3, SPINK2, HOPX, and others.

The Mast Cell and Basophil Progenitor subpopulation (MBPs) expresses genes such as TPSAB1, LMO4, HDC, MS4A2, KIT, and others, with absent expression of HBD.

The Eosinophil Progenitor subpopulation (Pro-Eosinophil) expresses genes like CLC, HDC, and others, with absent expression of MS4A2, TPSB2, and others.

The Monocyte-Macrophage Progenitor subpopulation (Pro-Mac) expresses genes such as EGR1, SPI1, KLF4, CEBPB, and others, with low or absent expression of CLEC9A, THBD, IRF8, and others.

The Monocyte-Dendritic Cell Progenitor subpopulation (Pro-DC) expresses genes like CLEC9A, ANPEP, IRF8, SPI1, and others, with absent expression of FCGR3A, CSF1R, and MAFB.

In this invention, the HPC populations were re-identified. The expression of specific genes enables the identification of each progenitor subpopulation. The specific gene expression or absence patterns are not limited to the listed genes but include other genes with similar expression profiles and cell lineage specificity.

The specific expression levels of genes—whether expressed, absent, or expressed at very low levels—within each of the aforementioned cell subpopulations are subject to minor variations depending on sample size and detection methodology. However, such variations do not compromise the defining characteristic that these subpopulations are collectively identified/redefined based on a multi-gene signature.

In one embodiment, the MEPs, Pro-ME, and MBPs all express specific genes, including CNRIP1, GATA1, KLF1, MYB, CDK4, KIT, and others, while showing low or absent expression of FLT3, SPINK2, HOPX, and CSF3R.

In another embodiment, the HPC Population also includes the Early Multipotent Hematopoietic Progenitor Subpopulation (MPCs), expressing genes such as AVP and CSF3R.

In a preferred embodiment, the MPCs can differentiate into three distinct directions:

Myeloid progenitor directions of megakaryocyte-erythroid lineage, expressing markers like GATA1, GATA2, and KLF1.

Lymphoid progenitor lineage, expressing markers like MME, CCR7, and IGHM.

Neutrophil and monocyte progenitor lineage, expressing markers like MPO.

In another preferred embodiment, the differentiation process (lineage commitment) of myeloid progenitor cells includes multiple stages, with the GAPs and NMPs emerging from the first priming stage. The GAPs progress through stages committing to MEPs, Pro-ME, and MBPs, while NMPs differentiate further into neutrophils and various types of Pro-Mac and Pro-DC progenitor cells.

In another preferred embodiment, the lymphoid progenitor lineage commitment process is divided into three stages:

First stage: Committed into the common lymphoid progenitor subpopulation (CLPs).

Second stage: Committed into Pro-NK, Pro-T, and Pro-B progenitor subpopulations

The third stage: The Pro-NK, Pro-T, and Pro-B progenitor subpopulations further differentiate into precursor cells of different types of lymphocytes.

In this invention, the characteristics of the HPC subpopulations include, but are not limited to, the expression profiles, types, quantities and proportions, differentiation directions, differentiation stages, differentiation pathways (trajectories), and the branches and nodes in these differentiation pathways of each progenitor subpopulation.

### Second Aspect

This invention provided a method for the preparation and identification of the aforementioned HPC populations. The method for preparing and identifying the HPC population provided by this invention includes the following steps: adding a LIN-negative removal system to a blood sample to remove non-hematopoietic stem cells from the sample, thereby obtaining the HPC population; capturing and sequencing single cells from the HPC population to obtain single-cell transcriptome data; performing unsupervised clustering analysis on the single-cell transcriptome data to identify the HPC subpopulations; wherein the LIN-negative removal system includes at least one of the following antibodies: CD3 antibody, CD19 antibody, CD56 antibody, CD11B antibody, CD16 antibody, CD36 antibody, CD66b antibody, CD61 antibody, and glycophorin A antibody.

In one preferred embodiment, the LIN-negative removal system may include one or more removal reagents or any combination thereof, such as first removal reagent, second removal reagent, third removal reagent, etc. Optionally, the removal reagents may include one or more of the following antibodies, or any combination thereof: CD3 antibody, CD19 antibody, CD56 antibody, CD11B antibody, CD16 antibody, CD36 antibody, CD66b antibody, CD61 antibody, and glycophorin A antibody.

In a specific embodiment, the first removal reagent may include the following antibodies: CD3 antibody, CD19 antibody, CD56 antibody, and glycophorin A antibody.

In another specific embodiment, the second removal reagent may include the following antibodies: CD11B antibody, CD14 antibody, or CD16 antibody.

In yet another specific embodiment, the third removal reagent may include the following antibodies: CD14 antibody, CD61 antibody, CD36 or CD41 antibody, CD66b antibody, and other antibodies.

Optionally, the multiple removal reagents may be added successively or simultaneously to the peripheral blood sample in any order.

In a preferred embodiment, the method may use the RosetteSep method in combination with the LIN-negative removal system to remove non-hematopoietic stem cells from peripheral blood, or may use immunomagnetic bead methods or flow cytometry sorting methods combined with the LIN-negative removal system to remove non-hematopoietic stem cells from peripheral blood.

In this invention, the blood sample may include peripheral blood, bone marrow blood, umbilical cord blood, or other common stem cell blood samples. The peripheral blood sample may include adult peripheral blood samples from the subject or peripheral blood samples mobilized by G-CSF.

In this invention, the identification included the re-identification and definition of the progenitor cell subpopulations in each lineage and differentiation stage using the expression profiles of transcription factors and characteristic marker genes. In a specific embodiment, the identification results contained newly identified genes and known marker genes with new lineage features and expression characteristics. In another specific embodiment, one or more characteristic gene combinations obtained by the identification method of the present invention can be used for re-identification and definition of progenitor cell subpopulations of various lineages at different differentiation stages and directions.

Preferably, the preparation method of this invention uses a negative enrichment method, which not only significantly increases the proportion of CD34-positive hematopoietic stem cells but also effectively reduces the loss of progenitor cell subpopulations, ensuring the integrity of subpopulations of progenitor cells at different differentiation stages.

Preferably, the present invention utilized adult peripheral blood as the sample source to enrich HSCs, distinguishing them from in vitro cultures, and obtained authentic progenitor cell subpopulations at various differentiation stages.

Preferably, this invention combined the detection of peripheral blood HSCs and G-CSF-mobilized peripheral blood HSCs, increasing detection proportion and obtaining a larger quantity of hematopoietic stem cells and progenitor subpopulations at different differentiation stages.

Preferably, the invention innovatively employed transcription factors to define and identify progenitor cell subpopulations, distinguishing them from surface protein marker genes, enabled more accurate definition and identification of progenitor cell differentiation stages and directions.

Preferably, the method of this invention also revealed the lineage evolution pathways and processes of hematopoietic stem cell lineage formation. The lineage evolution pathways included the differentiation directions, stages, paths, as well as the branches and nodes in the differentiation trajectories of various HPC subpopulations. The lineage evolution process encompassed the upstream and downstream progenitor cell subpopulations in the differentiation (lineage commitment) process.

### Third Aspect

This invention provided the expression profiles of fate-determining genes and marker genes for the HPC subpopulations of each lineage, which exhibited stage-specific and direction-specific differentiation characteristics.

The marker genes include, but are not limited to, the genes described in the “First aspect” above. The fate-determining genes include: the HPC group fate-determining genes, the common lymphoid progenitor subpopulation (CLPs) fate-determining genes, the NK progenitor subpopulation (Pro-NK) fate-determining genes, the T progenitor subpopulation (Pro-T) fate-determining genes, the B progenitor subpopulation (Pro-B) fate-determining genes, the plasma progenitor subpopulation (Pro-Plasma) fate-determining genes, the neutrophil and monocyte progenitor subpopulation (NMPs) fate-determining genes, the megakaryocyte-erythroid lineage progenitor (GAPs) fate-determining genes, the megakaryocyte-erythroid progenitor (MEPs) fate-determining genes, the megakaryocyte-erythroid precursor progenitor (Pro-ME) fate-determining genes, the mast and basophil progenitor subpopulation (MBPs) fate-determining genes, the eosinophil progenitor subpopulation (Pro-Eosinophil) fate-determining genes, the monocyte-macrophage progenitor subpopulation (Pro-Mac) fate-determining genes, and the monocyte-dendritic cell progenitor subpopulation (Pro-DC) fate-determining genes.

The fate-determining genes for the HPC group include the following genes: SOX4, CDK6, SERPINB1, FOXP1, SPI1, XBP1, ETV6, BCL11A, RUNX1, ERG, LMO2, CD82, CYTL1, EGFL7, NRIP1, IMPDH2, LY6E, ITGA4, SPINT2, EIF1, PPIA, PPIB, HMGB1, CD74, PFN1, TXN, ZFP36L2, CD37, HSP90AA1, and TMSB4X.

The fate-determining genes for the CLPs include the following genes: HOPX, DDIT4, HOXA9, and RUNX2.

The fate-determining genes for the Pro-NK include the following genes: DDIT4, HOPX, TBX21, and ID2.

The fate-determining genes for the Pro-T include the following genes: TCF7, GATA3, BCL11B, and DDIT4.

The fate-determining genes for the Pro-B include the PAX5 gene.

The fate-determining genes for the Pro-Plasma include the following genes: PRDM1 and IRF4.

The fate-determining genes for the NMPs include the following genes: MYB, CDK4, and CEBPA.

The fate-determining genes for the GAPs include the following genes: GATA2, NFE2, LYL1, and MYB.

The fate-determining genes for the MEPs include the following genes: GATA2, NFE2, LYL1, MYB, GATA1, KLF1, ZBTB16, TAL1, CDK4, and TESPA1.

The fate-determining genes for the Pro-ME include the CDK4 gene.

The fate-determining genes for the MBPs include the following genes: LMO4, CDK4, and MITF.

The fate-determining gene for the Pro-Eosinophil includes the ETV6 gene.

The fate-determining genes for the Pro-Mac include the following genes: SPI1, KLF4, CEBPB, EGR1, EGR2, CEBPA, MAFB, BCL6, and NR4A1.

The fate-determining genes for the Pro-DC include the following genes: SPI1, KLF4, IRF8, DDIT4, and BCL6.

The fate-determining genes included, but were not limited to, the genes listed above. They also included other genes with similar expression patterns, cell lineage specificity, or characteristic features.

Preferably, the genes contained newly identified genes as well as known genes with novel lineage-specific or expression-specific characteristics. These genes collectively participated in determining the differentiation stage and direction of progenitor cells. By activating or inhibiting one or more genes, precise control over the differentiation stage and direction of progenitor cells could be achieved.

In this invention, the expression profile possessed directionality, stage specificity, and characteristics. The directionality referred to the three main lineage commitment directions of the HPCs during the initial differentiation process: CLP, GAP, and NMP, as well as subsequent differentiation stages, branches, nodes, and paths (trajectories). The stage-specificity referred to the distinct differentiation stages characteristics across lineages, which exhibit continuous or stepwise characteristics. The expression profile characteristics described the transcriptional and functional attributes of marker genes and fate-determining genes, including presence or absence of gene expression, the expression level, the spatial distribution, dynamic changes in expression, and those corresponding to differentiation stage and direction in progenitor cells. Based on the expression profiles of specific, stage-specific, or absent genes—either individually or in combination with other known marker genes—accurate identification of the differentiation stages and directions of HPC lineages was achieved.

### Fourth Aspect

This invention provided a method for constructing the expression profile described in the “Third aspect” above.

The method for constructing the expression profile provided by this invention included the following steps: detecting the expression, expression levels, and characteristics of the marker genes (the genes described in the “First aspect”) and fate-determining genes (the genes described in the “Third aspect”) of the HPC subpopulations, and combining this with the characteristics of the HPC subpopulations to obtain the gene expression profile of the HPC subpopulations and their characteristics.

Specifically, the characteristics of the expression profile correspond to the characteristics of the HPC subpopulations. Based on the expression profile, it is possible to identify the differentiation stages, differentiation direction, and differentiation trajectories of the progenitor cell lineages. The dynamic changes (temporal), expression locations (spatial), and states of the expression profile can be clarified through the features of the progenitor cell lineages.

### Fifth Aspect

The present invention provided a method for identifying differentiation stages and fate-determining genes of HPCs, which comprising the following steps:
a) Enriching and identifying HPC populations at various differentiation stages and lineages;
b) Establishing expression profiles for each progenitor subpopulation;
c) Constructing dynamic correlations between fate-determining gene expression signatures and the progenitor subpopulations characteristics, wherein localization correlations include: gene expression levels, activation/inhibition states, and the differentiation direction, stage, and trajectory of progenitor cells;
d) Distinguishing differentiation stages, trajectories, branch points, and lineage hierarchy features among progenitor subpopulations of three distinct lineages—CLP, GAP, and NMP;
e) Constructing a trilineage hematopoietic hierarchy to identify differentiation stages and fate-determining genes of HPCs.

Step (1) included identifying intermediate transitional progenitor subpopulations (GAP and NMP) and their specific marker genes.

The method further comprised determining differentiation trajectories of each progenitor subpopulation and spatiotemporal features of fate-determining genes within the hematopoietic hierarchy.

Characteristic features of the method step (3) included identifying fate-determining genes activated or inhibited at different differentiation stages of progenitor cells.

Step (4) included mapping lineage directions and differentiation stages of each progenitor subpopulation within the hematopoietic hierarchy.

The method also included:

Identifying fate-determining genes regulating differentiation stages and directions of each progenitor subpopulation, and defining their spatiotemporal expression patterns and dynamic activation/inhibition features.

### Sixth Aspect

This invention provided a reconstructed hematopoietic hierarchy of human HPCs.

The hematopoietic hierarchy characteristics include:

The lineage features of said HPC populations include progenitor subpopulations that initially differentiate into three distinct lineages: CLP, GAP, and NMP.

The hematopoietic hierarchy of human HPCs contained two distinct stages in the megakaryocyte-erythroid lineage differentiation direction;

Late-stage differentiation of megakaryocyte-erythroid progenitors in the hematopoietic hierarchy exhibited two branches: a Pro-ME branch and a MBP branch;

The MBPs in the hematopoietic hierarchy demonstrated completely distinct differentiation branches and trajectories compared to NMPs;

The neutrophil differentiation trajectory in the human hematopoietic hierarchy is shortest with few stages, enabling rapid differentiation from progenitors to abundant mature neutrophils;

The HPC hierarchy identified transcription factors and fate-determining genes with lineage-specific and stage-specific characteristics.

The hematopoietic hierarchy clarified that the lineage commitment for megakaryocyte-erythroid lineages was continuous, while the lineage commitment of lymphoid lineages exhibited a stepwise progression.

The human hematopoietic hierarchy characterized two B-cell progenitor profiles: B-cell progenitors and plasma cell progenitors;

The hematopoietic hierarchy included features such as the differentiation stages, directions, and branches, and nodes, along with the corresponding expression profile features of characteristic genes and differentiation regulation features.

### Seventh Aspect

The present invention provided applications of the marker genes and fate-determining genes described in the first and third aspects for distinguishing or identifying hematopoietic progenitor cell subpopulations.

One or more gene combinations of HPC subpopulations were used to:
i. detect the quantity, state, and differentiation stage of single-lineage target progenitor cell subpopulations; or
ii. isolate and prepare high-purity target progenitor cell subpopulations.

Innovatively, unlike conventional cell detection methods, this approach not only determined cell types, quantities, and compositional ratios but also precisely identified the differentiation stage and direction of the cells.

### Eighth Aspect

The present invention provided applications of the marker genes and fate-determining genes described in the first and third aspects for sorting, enriching, or capturing HPC subpopulations, or detecting the number, status, or preparing high-purity progenitor cell subpopulations.

In a specific embodiment, the surface markers are selected from the genes described in the “First Aspect” or “Third Aspect”.

In a specific embodiment, the blood sample includes, but is not limited to, blood samples from healthy individuals, blood samples from individuals with blood diseases, immune or infection states, or from different physiological or pathological conditions.

In a specific embodiment, the detection results of the progenitor cell subpopulation's number and status may be used for diagnosing blood-related diseases, monitoring immune or treatment effects, and health status.

In a specific embodiment, the method further includes culturing and amplifying the sorted or enriched progenitor cells in a single culture, where the cultured and amplified progenitor cells can be used for cell transplantation to reduce immune rejection.

Preferably, based on the progenitor cell subpopulation lineage features, gene expression profiles, and hematopoietic hierarchy identified in this invention, combined with the method for distinguishing or identifying HPC subpopulations, it can achieve the sorting or enrichment of progenitor cells at different differentiation stages and directions.

### Ninth Aspect

The present invention provided the expression profile characteristics of the fate-determining genes for tracking and localizing progenitor cell differentiation stages.

Furthermore, the expression profile characteristics of the fate-determining genes were used to track and localization progenitor cell differentiation stages. Based on the expression patterns and dynamic changes of the fate-determining genes, the differentiation direction and stage of progenitor cells were tracked and localized.

The present invention utilized the expression profile characteristics of said genes for tracking and localization of the differentiation stages of hematopoietic stem cells and progenitor cells. By detecting or tracing the expression levels and dynamic changes of one or more genes corresponding to the target cells, the lineage differentiation stages and directions of hematopoietic stem cells and progenitor cells were tracked and localized.

The dynamic changes refer to the changes in the expression levels and characteristics of one or more marker genes or fate-determining genes associated with a specific hematopoietic progenitor cell subpopulation during its differentiation trajectory.

In this invention, the tracking and localization method also includes labeling lineage-specifically expressed genes via, through gene editing, fluorescent labeling, molecular tagging, or similar techniques. This allows for the detection of dynamic expression and changes of these markers during the differentiation process of the progenitor cells, thereby achieving progenitor cell lineage localization and tracking.

Preferably, based on the lineage-specific gene expression profiles, and hematopoietic hierarchy characteristics, combined with the method for tracking and localizing HPC subpopulations, the present invention can accurately track and locate the state, differentiation stage, and differentiation direction of progenitor cells.

### Tenth Aspect

The present invention provided applications of the expression profile characteristics of the fate-determining genes in progenitor cell reprogramming and regulation of hematopoietic progenitor cell differentiation or function.

Furthermore, the expression profile characteristics of the fate-determining genes were used to regulate the differentiation or function of hematopoietic progenitor cells. The functional regulation included regulating fate-determining genes of specific progenitor cell types, such as activating or inhibiting fate-determining genes, thereby achieving functional control over cell growth inhibition, killing, differentiation, and proliferation.

The expression profile characteristics of the fate-determining genes described in the present invention were used to regulate the differentiation trajectory or differentiation potential of hematopoietic stem cells and progenitor cells. Based on the expression profiles of fate-determining genes and hematopoietic hierarchy characteristics, the expression or activation of fate-determining genes in HPCs at specific differentiation stages or directions was modulated, thereby achieving precise regulation of differentiation or function in HSCs and HPCs.

In the present invention, the regulatory methods included, but were not limited to, the use of polypeptides, small-molecule drugs, or receptor molecules for modulation. The regulatory approaches included, but were not limited to, gene editing, gene transfection, etc. The regulatory process included, but was not limited to, temporal and spatial control of transcription factors and receptor gene activation or inhibition at different differentiation stages or directions, enabling accurate control over progenitor cell differentiation direction and stage.

In specific embodiments, regulating the differentiation stages of hematopoietic stem cells and progenitor cells involved modulating HPCs/HSCs at a particular stage, preventing further differentiation or maturation while preserving their pluripotent differentiation capacity. It also involved suppressing lineage-specific genes at differentiation stages to keep hematopoietic progenitor cells at their current differentiation stage without further differentiation or maturation; additionally, it encompassed preventing excessive differentiation.

In one embodiment, the method achieved suppression of blood tumor cell growth by introducing a substance (vector) that knocked out the HOPX gene into the tumor cells. The HOPX knockout substance could be any agent that functionally prevented the host cells from producing the HOPX gene's protein product. Typically, the knockout was performed at the genomic DNA level to ensure permanent inheritance of the knockout in subsequent cell generations. Preferably, the HOPX knockout substance was a Crispr cas9 vector designed to knockout the HOPX gene. The vector expressed a gRNA targeting the HOPX gene. The blood tumor cells included NK tumor cells (e.g., the NK92 cell line). More preferably, the target sequence of the gRNA was GACCGCGAGCGGCCCCACAG (Sequence ID No. 1).

The regulatory (intervention) approaches could be either activation or inhibition, achieved by overexpressing one or more of the aforementioned fate-determining genes in hematopoietic stem cells or somatic cells, or by suppressing the expression of one or more of these fate-determining genes. Specific methods included constructing lentiviral or adenoviral systems for transcription factor overexpression, lentiviral or adenoviral systems for transcription factor inhibition using small RNA, lentiviral or adenoviral systems for CRISPR-Cas9-mediated knockout of transcription factors via gRNA, small-molecule compound/drug-based activation or inhibition, co-culture activation, growth factor/cytokine stimulation, gene editing, and vector delivery—either individually or in combination.

The regulation of hematopoietic stem and progenitor cell differentiation trajectory specifically involved regulating differentiation across different stages or lineages; it also included regulation of progenitor cell differentiation based on all identified characteristic transcription factors and fate-determining genes.

The fate-determining genes described in this invention, when combined with progenitor cell characteristics, were used for temporally and spatially precise reprogramming of hematopoietic stem and progenitor cells with specific lineage differentiation potential. By integrating progenitor cell characteristics and hematopoietic hierarchy features, the lineage-specific and stage-specific fate-determining genes were engineered into multi-gene vectors and introduced into hematopoietic progenitor cells, pluripotent stem cells, somatic cells, or cell lines, thereby inducing reprogrammed cells with lineage-specific or multipotent differentiation capacity.

In this invention, the regulatory methods included, but were not limited to, designing multi-gene expression/suppression vectors, lentiviral systems, gene editing, small-molecule-activated gene combinations, and other approaches to obtain substances capable of reprogramming the corresponding progenitor cell types.

In one embodiment, the method involved introducing plasmids overexpressing GATA1, KLF1, and/or TAL1 into target cells to induce reprogrammed cells with erythroid characteristics. Specifically, the plasmids overexpressing GATA1, KLF1, and/or TAL1 were lentiviral vectors overexpressing these factors. The target cells included, but were not limited to, embryonic stem cells (ES), mesenchymal stem cells, or induced pluripotent stem (iPS) cells, such as HEK293T cells or human iPS (hiPS) cells. In a specific implementation, the induction process included culturing in embryoid body induction medium and erythroid induction medium. The embryoid body induction medium preferably contained differentiation-promoting factors such as BMP4, bFGF, and the small-molecule inhibitor Y-27632, while the erythroid induction medium preferably consisted of StemSpan™ SFEM II.

Preferably, based on the lineage-specific signatures, expression profiles, hematopoietic hierarchy features, combined with the characteristics of transcription factors and genes at different differentiation stages, nodes, and directions, the aforementioned reprogramming methods enabled temporally or spatially precise reprogramming of hematopoietic progenitor cells toward specific lineages and stages.

The applications of the fate-determining genes described in the first and third aspects also included optimizing the timing (e.g., specific differentiation stage) and dosage of growth factors or supplements required for progenitor cell induction culture, to achieve directed induction and differentiation control in in vitro progenitor cell culture systems. In one embodiment, based on the fate-determining gene expression profile characteristics of the target progenitor cell subpopulation, growth factors or supplements required for progenitor cell induction culture were identified, and the components, dosage, and timing of the induction culture system were determined, thereby optimizing directed induction and differentiation control in in vitro progenitor cell culture.

The main innovations of the invention addressed the following issues:

The enrichment problem of adult peripheral blood hematopoietic stem cells and progenitor cells, which was characterized by the rarity of cells, making it extremely difficult to obtain high proportions and all lineages of HPC subpopulations. The methods provided in this invention effectively solve this problem.

The identification of hematopoietic stem cells and progenitor cells had always been a challenge. Due to disputes over definitions and difficulties in identifying lineage subpopulations, the characteristics of various HPC subpopulations had never been clearly identified, representing a key obstacle in functional research, translational applications, cell transplantation, clinical applications. Based on the innovative and efficient enrichment and identification methods described above, the invention accurately and clearly identified key lineages of adult peripheral blood hematopoietic stem cells and the fate-determining genes of these lineages.

A new hematopoietic stem cell hierarchy was reconstructed, which precisely and clearly described the fate-determining factors involved in lineage branching and differentiation stages during hematopoietic stem cell lineage commitment, clarifying the lineage and differentiation trajectory of HPCs at each stage, as well as their differentiation processes. Based on the accurate, complete, and clear identification of progenitor cells, expression profiles, and the hematopoietic hierarchy, the invention enabled the application of progenitor cells, expression profiles, and hematopoietic hierarchy in various fields.

### BRIEF DESCRIPTION OF THE DRAWINGS

FIGS. 1A-1C show the analysis of the proportion of peripheral blood HSCs before and after enrichment.

FIG. 1A is the flow cytometry detection of enriched adult peripheral blood CD34-positive HSCs, with the proportion exceeding 30%. FIG. 1B is the single-cell sequencing clustering map showing the proportion of CD34-positive HSCs, where red dots represent positive cells. FIG. 1C is the statistical graph of the enrichment proportion of CD34-positive HSCs. The PBMC group is the non-mobilized group, with an average CD34-positive HSC proportion of 10.3%. The G-CSF group is mobilized adult peripheral blood, with an average CD34-positive HSC proportion of 46.1%.

FIGS. 2A-2D show the comparison of CD34+ cell proportions across samples, studies, and enrichment methods, detected at single-cell level. Red/blue dots indicate CD34+ cells.

FIG. 2A is the T-SNE map showing CD34+ cells enriched by LIN-negative depletion (same as FIG. 1B). FIG. 2B is the T-SNE map showing CD34+ cells from the GSE181989 study (PMID:35046994). FIG. 2C is the T-SNE map showing CD34+ cells enriched from cord blood by LIN-negative depletion. FIG. 2D is the T-SNE map showing CD34+ cells using other enrichment methods.

FIGS. 3A-3E show the comparison of HPCs distribution and expression profile.

FIG. 3A is the progenitor cell distribution map of the present invention, showing three distinct differentiation trajectories (arrows): myeloid, lymphoid, and neutrophil-monocyte lineages.

FIG. 3B is the UMAP map of control data GSE181989, showing limited cell data with scattered distribution. FIG. 3C is the UMAP map of control data GSE181989 and GSE117498, showing missing lineages and inconsistent data. FIG. 3D is the violin plots for marker genes (GATA2 in myeloid/MME in lymphoid progenitors) of the present invention, enabling clear subpopulation distinction. FIG. 3E is the violin plots for marker genes GATA2 and MME in control data GSE181989, showing poor subpopulation distinction.

FIG. 4 is the violin plots of marker genes for all stages of major hematopoietic progenitor lineages. Unsupervised clustering demonstrates that combinatorial markers (known and newly identified) can precisely classify and define each HPC subpopulation. Each subpopulation has several specific marker genes, indicating the enrichment-derived complete lineage integrity.

FIG. 5A is the UMAP plot of redefined lineages, displaying the three transitional progenitor subpopulations: CLPs, GAPs, and NMPs with distinct differentiation trajectories.

FIG. 5B is the distribution map (feature plots) of marker-positive cells enabling detection and distinguishing between HPC subpopulations.

FIG. 6 is the single-cell expression violin plots of HSC transcription factors and key genes, showing broad expression in all progenitor subpopulations.

FIG. 7 is the single-cell expression violin plots of widely expressed transcription factors and genes in HSCs: EIF1, PPIA, PPIB, HMGB1, CD74, PFN1, TXN, ZFP36L2, CD37, HSP90AA1, and TMSB4X.

FIGS. 8A and 8B are the expression maps of fate-determining transcription factors in myeloid progenitor lineages, revealing lineage-specific and stage-specific features.

FIG. 8A is the violin plots of KLF1, CEBPB (myeloid), and HOPX (lymphoid lineage, control). Results showed significant expression abundance (bubble color) and proportion (bubble size) of myeloid transcription factors NFE2 and GATA2 in myeloid subpopulations 2, 6, and 13 (FIG. 4), while lymphoid key gene HOPX has minimal expression in these subpopulations.

FIG. 8B is the dot plot of fate-determining transcription factors in myeloid progenitors. Early fate-determining factors (GTAT2, NEF2, LYL1, MYB, ETV6, RUNX1, TESPA1, CDK4) are co-expressed in myeloid-differentiating clusters C1 and C0; megakaryocyte-erythroid/mast cell lineage factors (TAL1, KLF1, GATA1, ZBTB16 solid-line boxe); monocyte/granulocyte lineage factors (EGR1, CEBPB, KLF4, SPI1 dashed-line box).

FIGS. 9A and 9B are the expression maps of fate-determining transcription factors in lymphoid progenitor lineages. FIG. 8A is the comparative analysis showing significantly elevated expression abundance and proportion of transcription factors in lymphoid progenitors (subpopulation 4) versus myeloid progenitors (subpopulations 2 and 6). This reveals stage-specific and lineage-specific features of transcription factors (e.g., high abundance/proportion of PRDM1 in progenitor subpopulation 9). FIG. 8B is the heatmap of gene set enrichment and key regulatory signaling networks in progenitor subpopulations. Here, X1 denotes subpopulation C1, X2 denotes C2, with other subpopulations labeled accordingly.

FIGS. 10A and 10B are composite figures of fate-determining gene expression in myeloid (MEPs) differentiation, including a heatmap and feature plots showing stage/direction distribution features of marker genes.

FIG. 10A is the heatmap of fate-determining genes for myeloid (MEPs) differentiation, showing high expression of megakaryocyte-erythroid progenitor lineage marker genes versus key marker genes in neutrophil-monocyte progenitor lineages. FIG. 10B is feature plots of marker gene expression distribution across stages/directions in progenitor differentiation-lineage commitment, revealing both stage and direction specificity.

FIGS. 11A and 11B are the heatmaps of fate-determining differential gene expression in (A) neutrophil-monocyte progenitor lineages (NMPs) and (B) common lymphoid progenitor lineages (CLPs), with distinct characteristic differences.

FIGS. 12A-12F show the qPCR validation of HSC/HPC-associated and lineage-associated marker genes in (FIG. 12A) PBMC populations and (FIGS. 12B-F) cell lines. FIG. 12C is the validation of MEP lineage-associated TFs. FIG. 12D is the validation of CLP lineage-associated genes. FIG. 12E is the validation of monocyte lineage-associated TFs. FIG. 12F is the validation of NK-associated TFs.

FIG. 13 is the violin plot of lineage-specific marker gene (partial) expression in hematopoietic progenitor subpopulations, showing only representatively redefined markers with distinct lineage and subpopulation specificity.

FIG. 14 is the single-cell definition and clustering t-SNE map of integrated third-party PBMC control group.

FIG. 15 is the violin plot of marker gene expression in PBMC control scRNA-seq data. Validation showing high consistency in expression characteristics between mature cell types and the present invention's redefined cell types, proving the accurate HPCs redefinition of the present invention.

FIG. 16 is the t-SNE map of reproducibility analysis across scRNA-seq replicates, showing consistent subpopulation distribution and lineage differentiation.

FIGS. 17A and 17B are the experimental results of hematopoietic progenitors transplantation in NSG mice.

FIG. 17A showing flow cytometry detection of the proportion of transplanted human CD19-positive cells.

FIG. 17B showing late-stage GVHD symptoms in transplanted mice.

FIGS. 18A-18E show the application validation of progenitor-specific receptor expression profiles in differentiation induction.

FIG. 18A is violin plot of receptor gene expression profiles in progenitor cells. FIG. 18B showing the CD61⁺ megakaryocyte proportion induced by receptor-matched growth factors (flow cytometry).

FIG. 18C showing the CD235a⁺ erythrocyte proportion induced by receptor-matched growth factors (flow cytometry). FIG. 18D is the red cell pellet after centrifugation of induced erythrocytes. FIG. 18E showing the proportion of CD61⁺ megakaryocytes and CD235a⁺ erythrocytes under joint induction, suggesting shared initial differentiation trajectory.

FIGS. 19A-19C showing flow cytometry results demonstrating proportional changes in CD71 and SLC40A1 expression during different erythrocyte induction stages, enabling differentiation stage localization and tracking (double labeling with CD235a).

FIG. 19A showing the proportion of CD71 and SLC40A1 cells in PBMCs (negative control). FIG. 19B showing the proportion of CD71⁺ and SLC40A1⁺ cell distribution at the early induction stage. FIG. 19C showing the proportion of CD71⁺ and SLC40A1⁺ distribution at the late induction stage.

FIGS. 20A-20C showing the sequence validation and functional analysis results of HOPX-knockout cells.

FIG. 20A showing sequencing verification of HOPX knockout efficiency using gRNA:
Upper: Partial sequence from Cas9-edited cells

(GCGGAGACCGCGAGCGGCCCCA, SEQ ID NO: 2)

Lower: Partial sequence from wild-type NK92 cells

(GGCGGAGACCGCGAGCGGCCCCA, SEQ ID NO: 3)

FIG. 20B showing inhibited cell growth post HOPX-knockout in lentivirus-transduced NK92 cells.

FIG. 20C is a violin plot showing lineage-specific gene expression profile for immunotherapeutic target screening and strategy development.

FIGS. 21A-21F are pictures of the designed multi-gene overexpression plasmid and lentiviral transduction results.

FIG. 21A is schematic of the multi-gene overexpression plasmid.

FIG. 21B showing lentiviral transduction efficiency in target cells.

FIG. 21C showing morphological features of cord blood-derived erythrocytes.

FIG. 21D is detection of reprogramming transcription factors expression during induction.

FIG. 21E showing the morphology of reprogramming-induced embryoid bodies.

FIG. 21F showing the proportion of CD235a⁺ cells after plasmid reprogramming.

FIG. 22 is a schematic representation of the classical hematopoietic hierarchy (left) and the reconstructed hematopoietic hierarchy of the present invention (right), providing a navigation map of lineage commitment trajectories, differentiation directions, and stages.

### DETAILED DESCRIPTION OF THE EMBODIMENTS

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, the preferred methods and materials are described herein. All publications and patent applications cited herein are incorporated by reference in their entirety. Nothing in this document should be construed as an admission that the present invention is not entitled to the grant of a patent by reason of prior invention, which may have been disclosed before the filing date of the present disclosure.

It should be noted that although terms such as “first,” “second,” etc., are used in the description and claims, these terms are only used to distinguish between different objects and are not intended to describe any specific order or sequence.

The term “hematopoietic progenitor cell population” as used in the invention refers to a population of cells that includes the common lymphoid progenitor subpopulation (CLPs), NK progenitor subpopulation (Pro-NK), T progenitor subpopulation (Pro-T), B progenitor subpopulation (Pro-B), plasma progenitor subpopulation (Pro-Plasma), neutrophil and monocyte progenitor subpopulation (NMPs), the first-stage megakaryocyte-erythroid progenitor subpopulation (GAPs), the second-stage megakaryocyte-erythroid progenitor subpopulation (MEPs), megakaryocyte-erythroid precursor progenitor subpopulation (Pro-ME), mast and and basophil progenitor subpopulation (MBPs), eosinophil progenitor subpopulation (Pro-Eosinophil), monocyte-macrophage progenitor subpopulation (Pro-Mac), and monocyte-dendritic progenitor subpopulation (Pro-DC). The term “common lymphoid progenitor subpopulation (CLP)” in the invention refers to a cell population that expresses the following genes: SPINK2, HOPX, HOXA9, RUNX2, LTB, IGHM, DNTT, PRSS2, SLC2A5, MME, CCR7, NKG7, LST1, CD79A, MZB1, BASP1, FLT3, and SPON1 at high levels, and expresses the following genes at extremely low levels or is negative for them: CNRIP1, FCER1A, GATA1, and S100A10. These genes constitute the marker genes for the lymphoid progenitor subpopulation.

The term “NK progenitor subpopulation” in the invention refers to a cell population that expresses the following genes: GNLY, NKG7, CD247, CCL5, FCGR3A, PRF1, GZMA, GZMB, KLRD1, KLRB1, KLRF1, CD3E, CD7, HOPX, IL2RB, TBX21, and ID2 at high levels, and expresses the following genes at extremely low levels or is negative for them: IL7R and GATA3. These genes constitute the marker genes for the NK progenitor subpopulation.

The term “T progenitor subpopulation” in the invention refers to a cell population that expresses the following genes: TCF7, IL7R, GATA3, KLRB1, CD3E, CD3D, CD7, CD247, LTB, BCL11B, and DDIT4 at high levels, and expresses the following genes at extremely low levels or is negative for them: GNLY, FCGR3A, and GZMA. These genes constitute the marker genes for the T progenitor subpopulation.

The term “B progenitor subpopulation” in the invention refers to a cell population that expresses the following genes: CD19, MS4A1, FCER2, CD79A, CD79B, IGHM, LTB, IGKC, PAX5, VPREB3, CD22, CD24, and FCRLA at high levels, and expresses the gene CD27 at extremely low levels or is negative for it. These genes constitute the marker genes for the B progenitor subpopulation.

The term “plasma progenitor subpopulation” in the invention refers to a cell population that expresses the following genes: CD27, CD38, IGKC, IGHA1, SLAMF7, CD79A, CD79B, PRDM1, IRF4, JCHAIN, and IFI30 at high levels, and expresses the following genes: MS4A1 and FCER2 at negative levels. These genes constitute the marker genes for the plasma progenitor subpopulation.

The term “NMP subpopulation” in the invention refers to a cell population that expresses the following genes: CSF3R, MPO, MGST1, IGLL1, S100A10, C1QTNF4, NPDC1, MYB, CDK4, CDCA7, CEBPA, and NPW at high levels, and expresses the following genes at extremely low levels or is negative for them: GATA2, SLC40A1, CNRIP1, and LTB. These genes constitute the marker genes for the neutrophil and monocyte progenitor subpopulation.

The term “GAP subpopulation” in the invention refers to a cell population that expresses the following genes: GATA2, NFE2, LYL1, MYB, SLC40A1, TESPA1, and CSF3R at high levels, and expresses the following genes at negative levels: GATA1 and KLF1. These genes constitute the marker genes for the megakaryocyte-erythroid lineage progenitor subpopulation.

The term “MEP subpopulation” in the invention refers to a cell population that expresses the following genes: GATA2, NFE2, LYL1, MYB, GATA1, KLF1, CSF2RB, and SLC40A1, but does not express the CSF3R gene. These genes constitute the marker genes for the megakaryocyte-erythroid progenitor subpopulation.

The term “megakaryocyte-erythroid precursor progenitor subpopulation” in the invention refers to a cell population that expresses the following genes: HBD, CDT1, MCM2, MCM6, MCM5, MCM4, MCM3, MCM7, CDCA7, CDK4, and TYMS at high levels, and expresses the following genes at extremely low levels or is negative for them: FLT3, SPINK2, HOPX, C1QTNF4, and CSF3R, and expresses the following genes at negative levels: MS4A2 and MS4A3. These genes constitute the marker genes for the megakaryocyte-erythroid precursor progenitor subpopulation.

The term “MBP subpopulation” in the invention refers to a cell population that expresses the following genes: TPSAB1, LMO4, HDC, MS4A2, TPSB2, MS4A3, KIT, PRG2, CLC, MCM2-MCM7, APOC1, MITF, and TRIB2 at high levels, and expresses the gene HBD at negative levels. These genes constitute the marker genes for the mast and basophil progenitor subpopulation.

The term “eosinophil progenitor subpopulation” in the invention refers to a cell population that expresses the following genes: CLC, HDC, RFLNB, MEIS1, and ETV6 at high levels, and expresses the following genes: MS4A2, TPSB2, and MS4A3 at negative levels. These genes constitute the marker genes for the eosinophil progenitor subpopulation.

The term “monocyte-macrophage progenitor subpopulation” in the invention refers to a cell population that expresses the following genes: EGR1, SPI1, KLF4, CEBPB, FCGR3A, CSF1R, CD68, CD86, ITGAX, FCGR2A, LYZ, LST1, EGR2, CEBPA, MAFB, TNF, BCL6, LILRB2, CD4, CD33, FCGR2A, IFI30, S100A9, NR4A1, HMOX1, C5AR1, and CD83 at high levels, and expresses the following genes at extremely low levels or is negative for them: CLEC9A, THBD, and IRF8. These genes constitute the marker genes for the macrophage progenitor subpopulation.

The term “monocyte-dendritic progenitor subpopulation” in the invention refers to a cell population that expresses the following genes: CLEC9A, ANPEP, THBD, IRF8, KLF4, CD68, CD86, ITGAX, LYZ, SPI1, LST1, DDIT4, SLAMF7, BCL6, BASP1, CD4, CD33, IFI30, and CD83 at high levels, and expresses the following genes: FCGR3A, CSF1R, and MAFB at negative levels. These genes constitute the marker genes for the dendritic progenitor subpopulation.

The “fate-determining genes” mentioned in the present invention refer to the genes that regulate the differentiation (lineage commitment) of HPC populations and various progenitor subpopulations. Specifically, the fate-determining genes that regulate the differentiation of HPC populations include SOX4, CDK6, SERPINB1, FOXP1, SPI1, XBP1, ETV6, BCL11A, RUNX1, ERG, LMO2, CD82, CYTL1, EGFL7, NRIP1, IMPDH2, LY6E, ITGA4, SPINT2, EIF1, PPIA, PPIB, HMGB1, CD74, PFN1, TXN, ZFP36L2, CD37, HSP90AA1, and TMSB4X; the fate-determining genes that regulate the differentiation of the CLP subpopulation in the opposite direction include HOPX, DDIT4, HOXA9, and RUNX2; the fate-determining genes that regulate the differentiation of the NK progenitor subpopulation include DDIT4, HOPX, TBX21, and ID2; the fate-determining genes that regulate the differentiation of the T progenitor subpopulation include TCF7, GATA3, BCL11B, and DDIT4; the fate-determining genes that regulate the differentiation of the B progenitor subpopulation include PAX5; the fate-determining genes that regulate the differentiation of the plasma progenitor subpopulation include PRDM1 and IRF4; the fate-determining genes that regulate the differentiation of the NMP subpopulation include MYB, CDK4, and CEBPA; the fate-determining genes that regulate the differentiation of the GAP subpopulation include GATA2, NFE2, LYL1, and MYB; the fate-determining genes that regulate the differentiation of the MEP subpopulation include GATA2, NFE2, LYL1, MYB, GATA1, KLF1, ZBTB16, TAL1, CDK4, and TESPA1; the fate-determining genes that regulate the differentiation of the Pro-ME subpopulation include CDK4; the fate-determining genes that regulate the differentiation of the MBP subpopulation include LMO4, CDK4, and MITF; the fate-determining genes that regulate the differentiation of the eosinophil progenitor subpopulation include ETV6; the fate-determining genes that regulate the differentiation of the monocyte-macrophage progenitor subpopulation include SPI1, KLF4, CEBPB, EGR1, EGR2, CEBPA, MAFB, BCL6, and NR4A1; the fate-determining genes that regulate the differentiation of the monocyte-dendritic progenitor subpopulation include SPI1, KLF4, IRF8, DDIT4, and BCL6.

The “signature genes” mentioned in the present invention include the aforementioned marker genes and the aforementioned fate-determining genes.

The “gene expression profile” mentioned in the present invention refers to the expression patterns (status) and dynamic characteristics of the signature genes associated with each progenitor subpopulation. This profile provided information regarding the expression of characteristic genes in a specific progenitor cell subset, including: presence/absence of expression, expression levels, and expression dynamics. Using this information, it is possible to identify or distinguish different progenitor subpopulations, or to sort or enrich hematopoietic progenitor subpopulations, or to detect the quantity and state, or prepare high-purity progenitor subpopulations, or to trace or locate specific progenitor subpopulations. Additionally, this information can be used to control the differentiation direction or potential of HSCs/HPCs through artificial regulation or intervention, or to reprogram the differentiation ability of specific or multiple lineages of hematopoietic stem cells and progenitor cells, or to optimize in vitro culture systems for HSCs/HPCs, or to inhibit the growth of progenitor cells and mature cell populations, or to control the proliferation of progenitor cells and mature cell populations, or to kill progenitor cells and mature cell populations, or to enhance the immune function of progenitor cells and mature cell populations, or to inhibit or regulate the growth of hematological tumor cells (reverse or alter the malignancy or biological behavior of hematological tumor cells in vitro), or to screen hematological tumor treatment drugs in vitro, or to construct in vitro drug screening models for hematological tumors.

The “hematopoietic hierarchy” mentioned in the present invention refers to the hierarchical structure characteristics of the hematopoietic progenitor cell populations, which are obtained by analyzing the re-identified and defined hematopoietic progenitor cell populations based on the expression profiles of the present invention. These hierarchical structure characteristics include the lineage features, differentiation direction, differentiation stages, differentiation pathways (trajectories), and branch points and nodes within the differentiation trajectories of the HPC populations.

The following detailed description of the invention, combined with specific embodiments, was provided to further clarify the invention. The embodiments given are for illustrating the invention and are not intended to limit the scope of the invention. The following examples can serve as guidelines for those skilled in the art to make further improvements, and in no way constitute a limitation on the scope of the invention.

The experimental methods in the following examples, unless otherwise specified, are standard procedures, conducted according to the techniques or conditions described in the literature of the field or as per product manuals. Materials, reagents, etc., used in the following examples, unless otherwise specified, are commercially available.

### Example 1: LIN-Negative Depletion Method for Enrichment and Identification of Human Peripheral Blood Hematopoietic Stem and Progenitor Cells

Traditional flow cytometry positive enrichment sorting and magnetic bead positive sorting for hematopoietic stem cells (HSCs) are commonly performed using bone marrow or umbilical cord blood, the stem cell state in umbilical cord blood is significantly different from that in adult hematopoietic stem cells. The proportion of hematopoietic stem cells in bone marrow is higher than in peripheral blood, and the previous studies have typically used bone marrow hematopoietic stem cells to identify and construct hematopoietic stem cell lineage trees. Although high-purity CD34-positive stem cells can be obtained, the main drawbacks of these methods include:

Based on surface markers, they cannot capture hematopoietic progenitor cell subpopulations with negative expression of stem cell surface marker proteins, resulting in the omission of key progenitor cell subpopulations.

Hematopoietic stem cells in bone marrow blood are typically in an undifferentiated (quiescent stem cell) state, making it difficult to enrich a full set of progenitor cells in various stages of differentiation of hematopoietic stem cells from blood.

In resting adult peripheral blood, hematopoietic progenitor cells are extremely scarce, and the number of effectively captured progenitor cells is minimal, preventing effective analysis.

Sampling of bone marrow hematopoietic stem cells is invasive and difficult, leading to a small sample size (usually only a few), with insufficient viable cell numbers.

Methods such as magnetic bead-based enrichment of CD34-positive hematopoietic stem cells have shown that CD34 RNA expression levels can exhibit partial negative expression in single-cell transcriptomics, leading to false-positive contamination in enrichment methods.

In view of the above, this example provides a method for enriching and identifying human hematopoietic stem cells and progenitor cells from peripheral blood. The method uses adult peripheral blood (donated by healthy volunteers from Shenzhen People's Hospital), healthy fetal umbilical cord blood, and peripheral blood mobilized with G-CSF (G-CSF-mobilized peripheral blood is obtained by mobilizing adult peripheral blood according to the following regimen: Granocyte G-CSF, 300 ug/12 hours, subcutaneously injected for 5 consecutive days, followed by peripheral blood collection) as test samples. The study was approved by the Ethics Review Committee of Shenzhen People's Hospital, and informed consent was obtained from the donors or guardians to evaluate the effectiveness of the enrichment method described in this invention. The hematopoietic progenitor cells described in this invention refer to hematopoietic stem cells at different stages of differentiation and are used in a general sense.

### 1. Method for Enriching and Preparing Human Hematopoietic Stem Cells and Progenitor Cells

1. Collect a test sample of 8 ml of blood using a sodium citrate anticoagulation tube, and transferred the blood into a 50 ml centrifuge tube.

2. Establish a LIN-negative depletion system. In this example, the RosetteSep method was used to remove non-hematopoietic cells, achieving the enrichment effect. Specifically, this method included a combination of one or more non-hematopoietic stem cell surface markers to form the LIN (CD3, CD19, CD56, CD11B, CD16, CD36, CD66b, glycophorin A, CD61, CD41, etc.) system, in combination with various removal schemes to remove non-hematopoietic stem cells in the blood, such as lymphocytes (CD3, CD19, CD20, CD56), myeloid cells including monocytes and granulocytes (CD11B, CD14, CD16), granulocytes (CD11B, CD66b), erythroid cells, and platelets (CD61, CD41, CD36, glycophorin A), etc. Alternatively, an immunomagnetic bead method for negative removal or a flow cytometry sorting method can be used in combination with the LIN-negative depletion system to remove the aforementioned non-hematopoietic stem cells, achieving the enrichment effect.

The specific LIN-negative depletion system and steps were as follows:

Preferably, the depletion system can be optimized by combining one or more of the following products: #15272, #15226, #15664, #15628, #15263, #15271HLA, #15026 (STEMCELL catalog numbers). Additionally, the use of reagents may be increased by 20% to optimize the removal of granulocytes and lymphocytes, which are typically in greater numbers. This example presented one combination scheme for reference, but different combinations may result in deviations in the proportion and population of enriched cells.

For each test sample tube, 50 μl/ml of Depletion Reagent A Cocktail (STEMCELL, catalog number 15272), 5 μl/ml of Depletion Reagent B Cocktail (STEMCELL, catalog number 15226), and 50 μl/ml of Depletion Reagent C Cocktail (STEMCELL, catalog number 15026) were added to the blood, mixed thoroughly, and incubated for 10 minutes.

Key components in Depletion Reagent A include the following antibodies: CD3, CD19, CD56, glycophorin A.

Key components in Depletion Reagent B include the following antibodies: CD3, CD19, CD11B, glycophorin A.

Key components in Depletion Reagent C include the following antibodies: CD2, CD3, CD14, CD16, CD19, CD24, CD56, CD61, CD66b, and glycophorin A. The Depletion Reagent C used alone serves as the control group.

3. After incubation, add PBS phosphate-buffered saline (SH30256.01, HyClone) equal to the blood volume to dilute it, mix thoroughly, and incubate for an additional 10 minutes.

4. Take a 50 ml centrifuge tube, add 15 ml of Ficoll (density gradient medium, GE, Cytiva), then layer the incubated and diluted blood sample on top of the Ficoll, let it stand for 5 minutes, and centrifuge at 1000 g for 30 minutes.

5. After centrifugation, transfer the remaining plasma layer and the enriched cell layer white membrane layer) to a new 50 ml centrifuge tube, then add 3-5 times the volume of PBS phosphate-buffered saline containing 0.04% BSA. Centrifuge at 800 g for 5 minutes at 4° C. to wash.

6. After centrifugation, discard the supernatant and resuspend the cell pellet in 100 μl. Add 1 ml of red blood cell lysis buffer (ACK, CS0001, Beijing Leagene Biotechnology Co., Ltd.) and lyse for 2-3 minutes. Then, add 5 times the volume of PBS phosphate-buffered saline to stop the lysis, and centrifuge at 800 g for 5 minutes at 4° C.

7. After centrifugation, discard the supernatant and resuspend the cell pellet in 50 μl. Perform cell counting, flow cytometry, or single-cell sequencing for identification.

### II. Flow Cytometry Identification of Human Hematopoietic Stem Cells and Progenitor Cells (CD34)

1. Take the hematopoietic stem cell suspension enriched in Step I and transfer it into a 15 ml centrifuge tube. Centrifuge at 300 g for 5 minutes. After centrifugation, discard the supernatant, wash once by adding 1 ml of PBS phosphate-buffered saline containing 0.04% BSA. Centrifuge at 300 g for 5 minutes, discard the supernatant, and resuspend the pellet in 60-80 μl of the final volume. Mix well.

2. Take two 1.5 ml EP tubes, add 30 μl of the cell suspension to each tube, serving as the blank control group.

3. Add the remaining cell suspension to another 1.5 ml EP tube, which will be used for the staining group. First, add 2 μl of blocking solution FC (Biolegend) to block for at least 10 minutes.

4. After blocking, add 3 μl of APC-CD34 antibody (Biolegend) and mix thoroughly. Incubate in the dark on ice for approximately 1-2 hours.

5. After incubation, add 1 ml of PBS phosphate-buffered saline containing 0.04% BSA to both the staining group and the blank control group EP tubes. Mix well, then transfer the mixed cell suspensions into two flow cytometry tubes for further analysis.

6. Turn on the analytical flow cytometer and open the CytExpert software. Perform a cleaning process according to the startup procedure.

7. After cleaning, select the corresponding fluorescence channel based on the staining, and plot the dot plot and histogram.

8. Adjust the sample injection speed, usually medium speed. If there are fewer cells, adjust it to high speed.

9. After the blank control group is loaded, observe the dot plot. Adjust the FSC and SSC values so that P1 can encompass more than 90% of the cells.

10. Observe the histogram. When the peak of the histogram is centered, adjust the values to move the peak to the left, and fine-tune it until the peak curve is reasonable.

11. Based on the analysis, gate the target cell population and record the percentage of positive cells.

### III. Proportion Analysis of Hematopoietic Stem Cells Before and After Enrichment

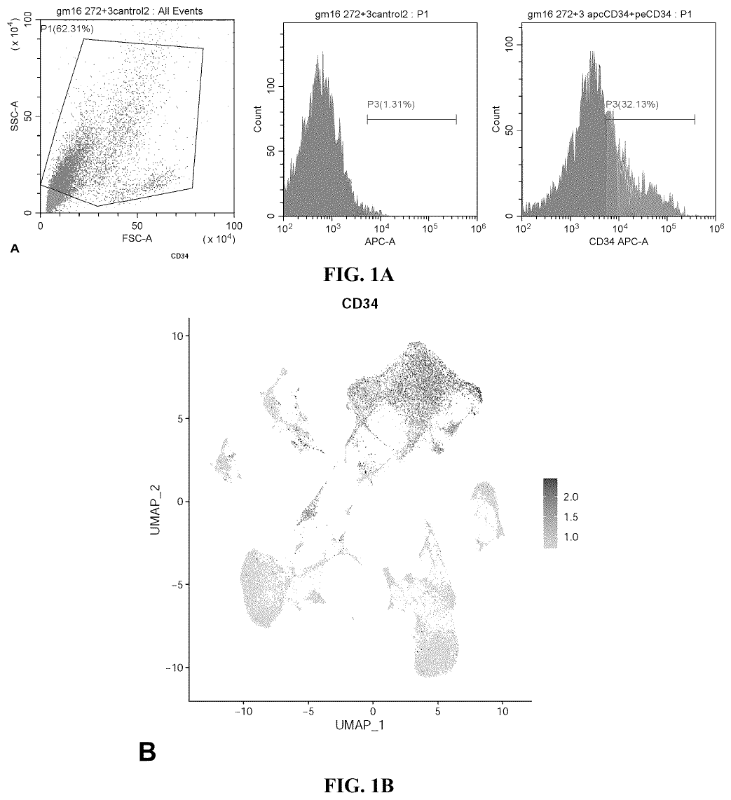

The proportion of peripheral blood CD34-positive hematopoietic stem cells is relatively low (usually less than 1%) by conventional enrichment methods. The results show that after enrichment using the LIN-negative system of the present invention, the average proportion of peripheral blood CD34-positive hematopoietic stem cells increased to 10.3%. The most obvious increase is observed in G-CSF-mobilized peripheral blood, where the proportion of CD34-positive cells significantly increases to more than 30% (FIG. 1A). Using multiple detection methods, such as single-cell sequencing (FIG. 1B), the proportion of CD34-positive cells is significantly increased (data compared to other studies, are detailed in Example 3). Statistical analysis shows that the proportion of CD34-positive hematopoietic stem cells in peripheral blood mononuclear cells (PBMCs) is on average 10.3%, while the average proportion in mobilized peripheral blood is 46.1% (FIG. 1C). Both peripheral blood and mobilized peripheral blood show a significant increase in enrichment, indicating that the enrichment method of the present invention can efficiently increase the proportion of CD34-positive hematopoietic stem cells in adult peripheral blood.

This example successfully addresses the longstanding challenge of enriching hematopoietic stem/progenitor cells from adult peripheral blood, with the following advantages:
a) The enrichment efficiency is significantly improved, with the average CD34-positive cell rate in adult peripheral blood reaching 10% (FIGS. 1A-1C).
b) Negative enrichment method reduces the loss of progenitor cell subpopulations, ensuring the integrity of progenitor cell subpopulations at different differentiation stages.
c) Enrichment of adult peripheral blood hematopoietic stem cells results in the acquisition of real progenitor cell subpopulations at different differentiation stages (distinct from in vitro cultivation).
d) Combined detection of peripheral blood hematopoietic stem cells and their mobilized counterparts significantly improved the efficiency of hematopoietic stem cell identification, enabling the acquisition of increased hematopoietic stem cell subpopulations (progenitors) at distinct differentiation stages.

### Example 2: Single-Cell Capture and Sequencing of Human Peripheral Blood Hematopoietic Stem Cells

Based on the high proportion of hematopoietic stem/progenitor cells suspensions obtained from adult peripheral blood and mobilized peripheral blood in Example 1, single-cell transcriptome sequencing was performed on all stem/progenitor cell subpopulations to capture the full transcriptomic profile. The detailed steps for single-cell capture and sequencing are as follows (10× Genomics, Standard Protocol CG000330 Rev A, V2):

1. Take the enriched hematopoietic stem/progenitor cell suspension and resuspend in PBS phosphate-buffered saline containing 0.04% BSA for counting. Adjust the cell concentration to approximately 2000 cells/μl.

2. Prepare the Master Mix (PN-1000266) on ice according to the number of samples. The volume per well is 36.3 μl, and the specific components are as follows:
RT Reagent B: 18.8 μl; Poly-dT RT Primer: 7.3 μl; Reducing Agent B: 1.9 μl; RT Enzyme C: 8.3 μl

3. Add appropriate nuclease-free water and corresponding volume of the single-cell suspension (up to 38.7 μl) to the Master Mix, and the total volume of the mixture is 75 μl in each well.

4. Mix thoroughly, then transfer 70 μl of the mixture into the sample wells of the chip. Dispense an equal volume of 50% Glycerol (Ricca Chemical Company) into unused Chip Wells.

5. Vortex and mix the gel beads thoroughly, then aspirate 50 μl of the gel beads and dispense into the bead wells of the chip. Dispense an equal volume of 50% glycerol into unused chip wells.

6. Add 45 μl of Partitioning Oil to the Oil wells of the chip. Dispense an equal volume of 50% glycerol into unused chip wells.

7. Seal the gasket, ensuring that the holes align with the wells.

8. Open the 10× Single-Cell Controller, place the assembled chip with the gasket in the tray, ensuring that the chip stays horizontal. Confirm the Chromium Chip K program on screen. Press the play button.

9. After the run is complete, remove the chip, discard the gasket, and open the chip holder. Fold the lid back at a 45-degree angle to expose the wells. Check if there are any abnormalities during the run. If everything is normal, slowly recover 100 μl of GEMs (Gel Beads in Emulsion) from the magnetic bead GEM wells.

10. Aspirate the GEMs sample and perform PCR reverse transcription (125 μl reaction system) with the following program: 53° C. for 45 minutes; 85° C. for 5 minutes; Store at 4° C.

11. After PCR reverse transcription, perform library preparation for the sample, resulting in single-cell cDNA samples.

12. Based on the 10× Genomics platform (Chromium Next GEM Single Cell 5′ GEM Kit v2), construct the library and perform next-generation sequencing to obtain the raw sequencing data and information of the single-cell transcriptome expression profile of all hematopoietic stem cell subpopulations. This single-cell transcriptome data will be used in Example 3 for data analysis to identify the hematopoietic stem cell and progenitor cell lineages and their marker genes.

### Example 3: Definition of Hematopoietic Stem Cell and Progenitor Cell Lineages in Human Peripheral Blood and Identification of Differentiation Stages

Due to the scarcity and heterogeneity of HSCs in adult peripheral blood, and the complexity of their subpopulations, it is currently difficult to effectively identify and define all subpopulations and progenitor cells at different differentiation stages.

Although single-cell sequencing technologies have been used for lineage detection and identification of HSCs, there are several shortcomings in the current studies:

The number of hematopoietic stem cells is small, and most studies use bone marrow samples.

Surface markers of hematopoietic stem cells are often used for capture and enrichment via immunomagnetic beads or flow cytometry, resulting in the loss of subpopulations at different differentiation stages.

Due to the small number of cells, classification and identification of stem cells are still based on surface markers such as CD38, FLT3 (CD135), and KIT (CD117), meaning the classification remains somewhat subjective.

In view of these issues, this invention utilizes the single-cell transcriptome expression profile sequencing data obtained from peripheral blood hematopoietic stem/progenitor cell subpopulations in Example 2. Unsupervised clustering analysis was performed on the single-cell transcriptome data to obtain the lineage subpopulations of human hematopoietic stem/progenitor cells and progenitor cell lineages at different differentiation stages. Additionally, the accuracy of progenitor cell definition and identification was validated by newly identified marker genes as well as known lineage marker genes.

### Advantages of this Example

A total of 27,566 hematopoietic stem/progenitor cells were successfully captured.

Successfully captured progenitor cell subpopulations at various differentiation stages of the lymphoid and myeloid lineages.

The atlas of progenitor cell subpopulations at different differentiation stages, including marker genes and transcription factors, was obtained at the single-cell level.

In contrast to other research methods for clustering and identifying hematopoietic stem cells, such as classifying lymphoid and myeloid cells using traditional definitions based on stem cell surface markers like CD49f, CD38, FLT3 (CD135), KIT (CD117), this example redefines and identifies more accurate and reliable hematopoietic stem/progenitor cell lineages and progenitor cell subpopulations based on an unsupervised clustering approach, which is based on the gene expression profile of the CD34-positive cell subpopulation and excludes human interference.

### I. Expression Profile Data Analysis of Human Peripheral Blood Hematopoietic Stem Cells and Progenitor Cells

The specific steps for single-cell transcriptome data analysis were as follows:

1. Quality control and filtering of next-generation sequencing raw data: The shell code was as follows:

Bash: fastp -I file.dir/sample.R1.fastq.gz -o
file.dir/sample.R1.fastq.gz -I / file.dir/sample.R2.fastq.gz -O
file.dir/sample.R2.fastq.gz

2. Data alignment by Cellranger:

Bash: cellranger count --id=sampleID
--transcriptome=dir/refdata-gex-GRCh38-2020-A --sample= sampleID,
sampleID -2 --localcores=12 --fastqs=dir/raw

3. Bioinformatics analysis: The filtering, dimensionality reduction, and cell annotation analysis of single-cell data were conducted using the R language analysis package: Seurat. The analysis methods and processes are well-known in the field. Standard analysis code and workflows are followed to ensure consistent results without special handling.

4. Extract all CD34-positive cells for unsupervised analysis and reclustering:

cd34.subset<-subset(data, CD34>=1, slot=“counts”)

5. Gene expression map plotting: Gene expression map was plotted using the VlnPlot, heatmap, and bubble plot functions. Some gene names have the prefix rna_due to software and plotting function requirements. The gene names mentioned in this invention were based on annotations and analysis from Cellranger, Seurat, and other software. The reference database for gene name annotations was GRCh38-2020 and refdata-gex-GRCh38-2020-A version 32 (Ensembl 98), with analysis software and version as cellranger-5.0.1.

Comparison of Enrichment Effect of Different Experimental Methods, Sample, and studies:

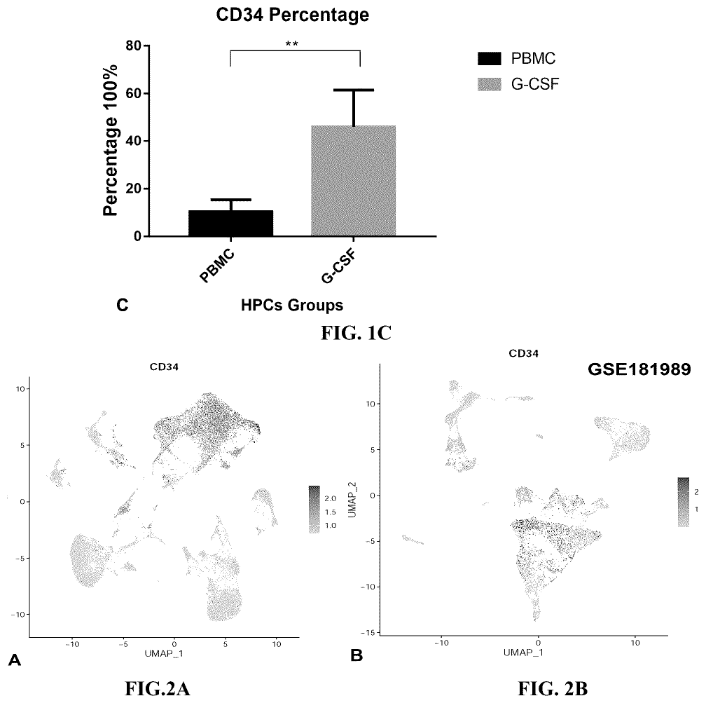

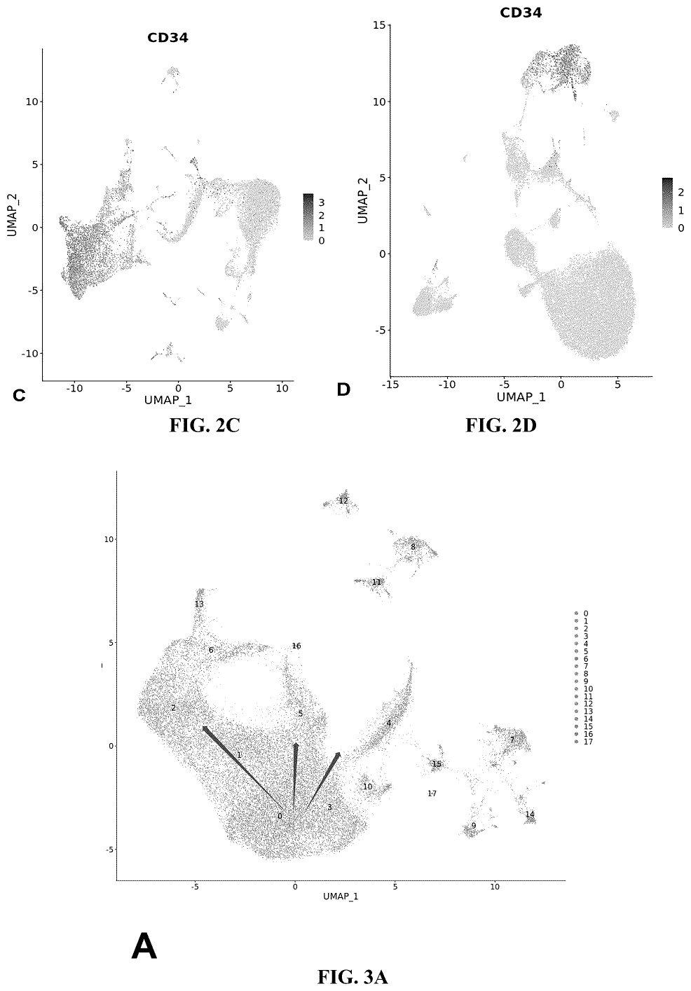

Single-cell sequencing results: The single-cell sequencing shows that the hematopoietic stem cell enrichment method using the LIN-negative depletion method from Example 1 increased the proportion of CD34-positive stem cells to more than 30% (FIG. 2A, same as FIG. 1B). When comparing the CD34 cell proportions from our enrichment method with single-cell data from the GSE181989 study (PMID: 35046994, GSE181989), the following results were obtained (FIG. 2B):
GSE181989: 1103/8904=12.4%; Our method: 6813/22174=30.7%

This comparison shows a significant improvement in CD34 cell enrichment proportion using this invention's method.

Cord blood enrichment: Using the same experimental protocol and process, the CD34-positive cell proportion in the enriched cord blood is 35.0% (FIG. 2C). When comparing this with the enrichment method using Deletion Reagent D (STEM CELL, 15026), which yields a CD34-positive cell proportion of only 6.3% (FIG. 2D), this invention's method demonstrates a higher proportion of CD34-positive cells and more complete cell subpopulations.

### Conclusion

From the above comparisons, it is evident that this invention's enrichment method significantly outperforms the GSE181989 study and other enrichment methods (e.g., STEM CELL, 15026). Furthermore, this method works well for other common stem cell blood sample types, such as cord blood, and provides consistently good enrichment results.

### II. Identification of Hematopoietic Progenitor Cell Subpopulations at Different Differentiation Stages in the Lymphoid and Myeloid Lineages

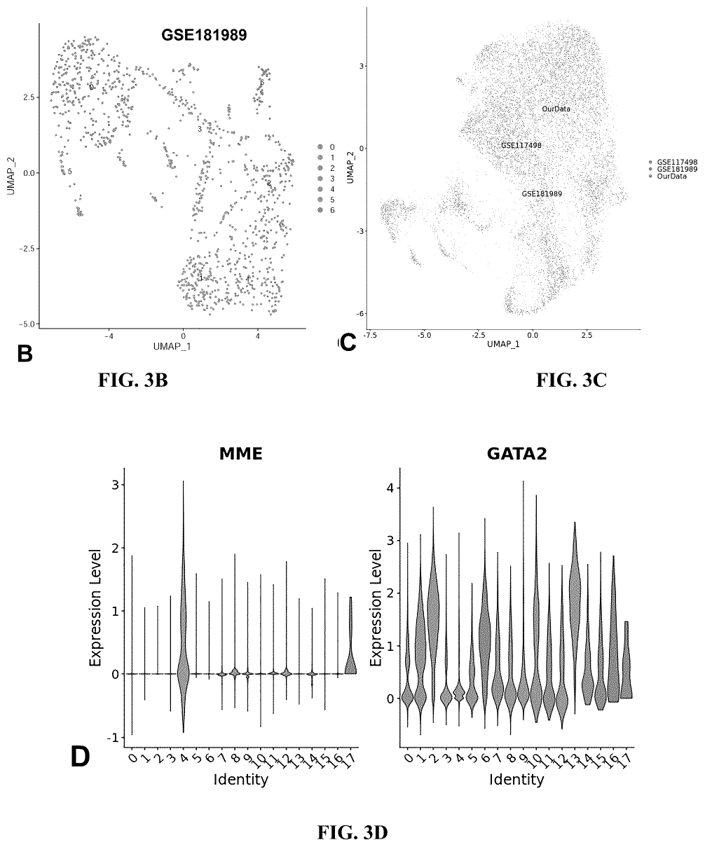

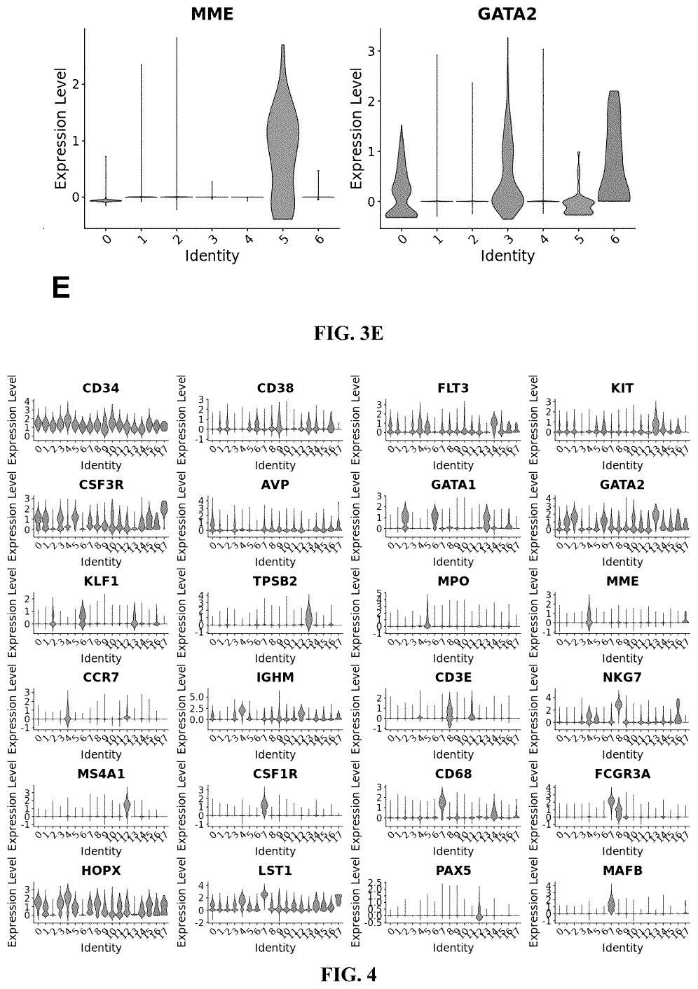

The subpopulations of progenitor cells enriched by this invention contain sufficient numbers, with key subpopulations having a total of over 3,000 cells. The hematopoietic progenitor cells follow a regular distribution pattern, showing distinct differentiation stage differences and directionality. Each progenitor cell subpopulation displays typical features with clear distinctions (FIG. 3A). The single-cell transcriptome data obtained from this invention (FIG. 3A) was compared with two published HSCs-related datasets available in the NCBI GEO database, which contained larger numbers of CD34-positive cells (GSE181989 with 1,103 cells, GSE117498 with 3,393 cells), for comparing the hematopoietic progenitor cell lineage transcriptomic atlas.

The results shows that although GSE181989 used bone marrow samples with the highest proportion of hematopoietic stem cells and captured both myeloid and lymphoid progenitor cells, the effective cell data was very limited (only 1,103 CD34-positive cells), and the distribution was scattered (FIG. 3B). The expression of GATA2 and MME within the same subpopulation made it impossible to distinguish the subpopulations effectively (FIG. 3E). GSE117498 used magnetic bead-sorted bone marrow CD34-positive cells, but it exhibited the loss of key differentiation lineages (FIG. 3C); the blue cell subpopulations in the lower left corner was not enriched by other studies (FIG. 3C), while the main cell subpopulations in the upper right, which other studies had enriched (red dots), were present in very small proportions. Inversely, the progenitor cell marker genes and characteristic expression profiles of each subpopulation enriched by this invention can clearly distinguish progenitor cell subpopulations at different differentiation stages (FIG. 3D, FIG. 4), such as GATA1 and KLF1 being expressed only in subpopulations 2, 6, and 13, while MME is specifically expressed in subpopulation 4. The expression of specific and characteristic marker genes shown in FIG. 4, based on objective gene expression profile characteristics, eliminates subjective bias and effectively distinguishes different types of progenitor cells. This enables accurate and reliable definition and identification of hematopoietic progenitor cell subpopulations at different differentiation stages in the lymphoid and myeloid lineages.

The gene expression profile in this example indicates that classic hematopoietic stem cell lineage marker genes such as CD38, FLT3, and KIT show scattered expression (FIG. 4), without significant subpopulation or differentiation stage specificity, making it impossible to effectively classify and identify progenitor cell subpopulations, especially for progenitor cell subpopulations at different differentiation stages, where the differentiation and identification efficacy is low (FIG. 4). Therefore, based on the hematopoietic stem cell and progenitor cell lineage marker genes, this example selects clear, well-known, and widely recognized other lineage marker genes, in combination with newly identified characteristic marker genes, to redefine and identify all hematopoietic progenitor cell lineages. The results show that in the unsupervised clustering expression profile established with the 27,566 CD34-positive single-cell data obtained from the enrichment, multiple specific marker genes are expressed in each subpopulation, and the cell types identified by some marker genes are consistent with classical cell type definition genes (e.g., MME, MS4A1, CD68). New identified marker genes are highly specific (e.g., GATA1, KLF1, HOPX, PAX5), effectively distinguishing progenitor cell subpopulations at different differentiation stages and directions. Specifically (FIG. 4), subpopulation C0 expresses early progenitor cell marker genes AVP and CSF3R, identified as early multipotent progenitor cells (MPCs). These early progenitor cells then differentiate into three distinct directions: Differentiation Direction 1 (FIG. 3A, subpopulation C0->C1->C2->C6 and C13) corresponds to the megakaryocyte-erythroid differentiation direction of myeloid progenitor cells, expressing typical marker genes such as GATA1, GATA2, and KLF1, with the expression trend of the classic myeloid progenitor cell marker gene KIT being consistent with this direction (FIG. 4). GATA1 and KLF1 are known classic markers and key transcription factors for megakaryocyte-erythroid differentiation, and the expression map shows that GATA1 and KLF1 are specifically expressed in megakaryocyte-erythroid progenitor subpopulations at different differentiation stages (subpopulations 2, 6, and 13), with early megakaryocyte-erythroid progenitors (subpopulation C1) not expressing them and showing negative expression in other subpopulations. The results suggest that based on GATA1 and KLF1 as marker genes, not only can we identify the positive-expressing cell subpopulations as megakaryocyte-erythroid progenitor cells, but we can also effectively distinguish different differentiation stages of megakaryocyte-erythroid progenitors. Differentiation Direction 2 (FIG. 3A, subpopulation C0->C3->C4) corresponds to the differentiation direction of lymphoid progenitor cells, expressing typical marker genes MME, CCR7, and IGHM. Differentiation Direction 3 (subpopulation C0->C5) corresponds to the neutrophil-monocyte differentiation direction, expressing typical neutrophil marker genes such as MPO.

This example innovatively uses transcription factors to define and identify progenitor cell subpopulations, which, unlike surface protein marker genes, enables more accurate definition and identification of the differentiation stage and direction of progenitor cells. Furthermore, the expression levels, expression distributions, and dynamic changes of these lineage-specific markers exhibit precise alignment with the respective progenitor cell types and their stage-specific characteristics. For example, GATA1 is highly expressed only in the megakaryocyte-erythroid progenitor subpopulations C2 and its subsequent subpopulations (expression level and distribution), and its expression level increases as the cells gradually differentiate (dynamic changes) (FIG. 4); FLT3 is negatively expressed in the megakaryocyte-erythroid differentiation direction (level and distribution); the expression of CSF3R gradually decreases and eventually disappears as the megakaryocyte-erythroid differentiation progresses (dynamic changes) (FIG. 4). In this embodiment, combining the progenitor cell lineage features, the kinetic profiles of gene expression (time), expression locations (spatial distribution), and states are clearly defined.

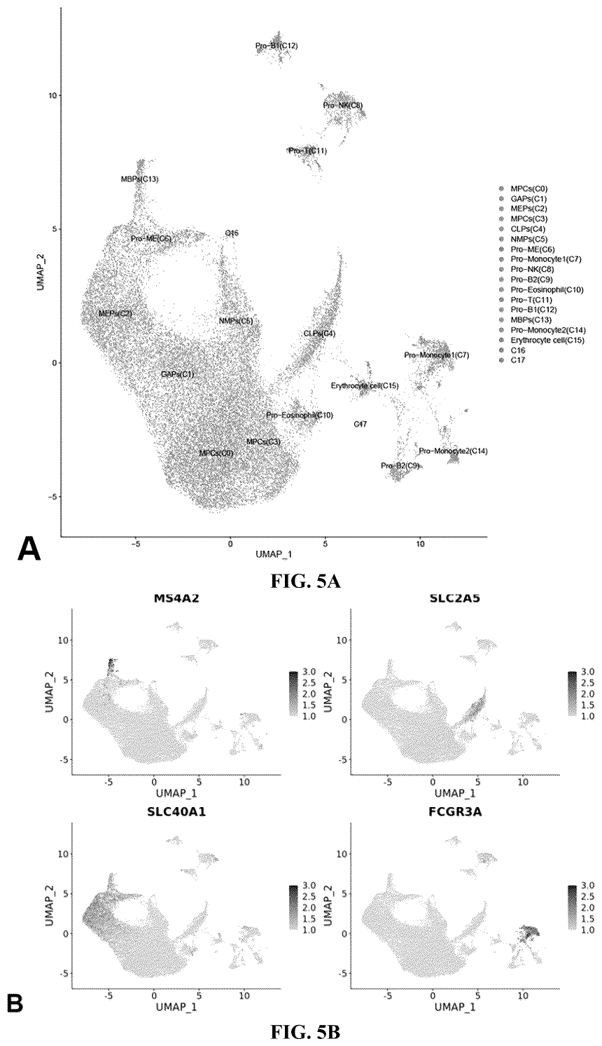

Based on the identified marker genes (Table 1 of Example), the entire hematopoietic progenitor cell lineage was redefined and identified. The specific redefinition names are as follows (FIG. 5A): Hematopoietic progenitor cells C0 and C3 subpopulations (multi-potent progenitors cells, MPCs), common lymphoid progenitor C4 subpopulation (CLPs), and T progenitor subpopulation cells (C11, Pro-T), plasma progenitor cells C9 subpopulation (Pro-B2/Pro-Plasma), NK progenitor cells C8 subpopulation (Pro-NK), and B progenitor cells C12 subpopulation (Pro-B1/Pro-B); the first-stage megakaryocyte-erythroid progenitor C1 subpopulation (GATA2 genes controlled progenitors, GAPs) and the second-stage megakaryocyte-erythroid progenitor C2 subpopulation (megakaryocytic-erythroid progenitors, MEPs), mast and basophil progenitor C13 subpopulation (mast cell or basophil progenitors, MBPs), megakaryocyte-erythroid precursor C6 subpopulation (megakaryocytic and erythroid precursor, Pro-ME); neutrophil and monocyte progenitor C5 subpopulation (neutrophilic and monocyte progenitors, NMPs) and monocyte-macrophage progenitor C7 subpopulation (Pro-monocyte1/Pro-Mac), and monocyte-dendritic progenitor C14 subpopulation (Pro-monocyte2/Pro-DC), eosinophil progenitor C10 subpopulation (Pro-Eosinophil).

This example redefined and identified the hematopoietic progenitor cell lineage subpopulations at different differentiation stages and directions in the lymphoid and myeloid lineages, specifically as follows:

Initial differentiation stage of hematopoietic progenitor cells: MPCs differentiate into three directions simultaneously. Under the regulation of GATA2, CSF3R, and others, the first differentiation branch in the myeloid differentiation direction appears, including megakaryocyte-erythroid progenitor cells (GAPs) and neutrophil and monocyte progenitor cells (NMPs). Under the regulation of HOPX, they differentiate into common lymphoid progenitor cells (CLPs).

Myeloid progenitor cell differentiation process: This process can be divided into three stages. The invention did not identify common myeloid progenitor cells (CMPs). In the first stage, they differentiate into GAPs and NMPs. The second stage corresponds to the MEP phase, after which MEPs differentiate into MBPs and Pro-ME as distinct subpopulations. NMPs subsequently differentiate into neutrophils and various types of monocyte-macrophage subpopulations.

Lymphoid progenitor cell differentiation process: This process can be divided into three stages. In the first stage, CLPs are differentiated, which further differentiate into T, B, and NK progenitor cells. T, B, and NK progenitor cells then differentiate into various types of lymphocyte precursor cells.

Clarification of the hematopoietic stem cell lineage evolution path: The process is characterized by defined differentiation stages and directions. These stages can be distinguished via specific marker genes, enabling precise identification and separation of cell subpopulations (FIG. 5B). The embodiment identified branching nodes, differentiation paths, and upstream/downstream progenitor cell groups within the differentiation process (FIG. 5A), thereby delineating the lineage evolution path.

### III. Identification of Fate Determining Transcription Factors and Key Genes for Progenitor Cell Subpopulations at Different Differentiation Stages

Since the method for inducing stem cell differentiation using the transcription factors Oct3/4, Sox2, c-Myc, and Klf4 was established by Shinya Yamanaka in 2006, various manipulations, such as overexpression, inhibition, and activation of these transcription factors, have been widely recognized for feasibility and practicality in controlling or inducing stem cell differentiation and reprogramming.

This embodiment is based on the effectively enriched hematopoietic progenitor cells (Example 1), and successfully defines and identifies the key lineage subpopulations of hematopoietic progenitor cells, their transcriptomic expression profiles (Example 2 and this embodiment), and the expression characteristics of differentiation directions and stages (spatial-temporal expression characteristics). Key transcription factors and genes involved in lineage fate determination were identified and categorized into the following three groups:

### First Category: Key Transcription Factors and Regulatory Genes for Hematopoietic Stem Cells

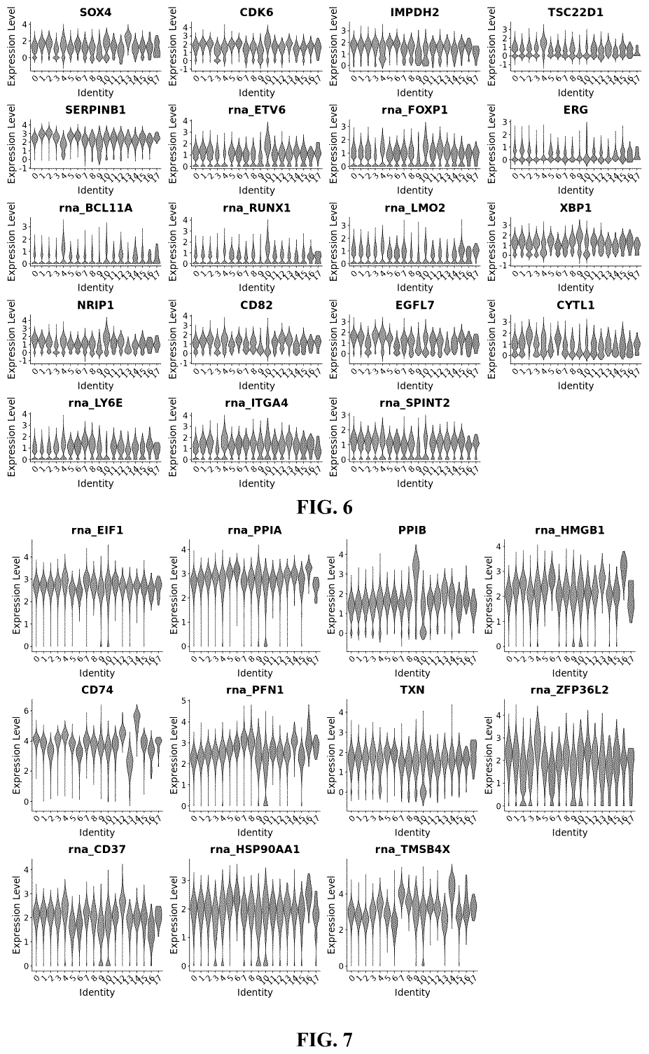

The key transcription factors include SOX4 (ENSG00000124766), CDK6, SERPINB1, FOXP1, SPI1, XBP1, ETV6, BCL11A, RUNX1, ERG, LMO2, and key regulatory genes include CD82, CYTL1, EGFL7, NRIP1, IMPDH2, LY6E, ITGA4, SPINT2. SPI1 (PU.1) is a known key transcription factor for hematopoietic stem cells. The identification criteria are as follows: these transcription factors are widely expressed in all progenitor cell subpopulations but are rarely or not expressed in mature cells. These genes exhibit stem cell expression specificity, with particularly high levels in early HSCs and early-stage progenitor cells (C0, C1, and C2 clusters). The expression profiles are shown in FIG. 6, where ETV6 and SERPINB1 exhibit myeloid progenitor cell bias, and SPI1 exhibits monocyte-macrophage progenitor cell bias.

These key transcription factors and genes were widely expressed in HPCs, including EIF1, PPIA, PPIB, HMGB1, CD74, PFN1, TXN, ZFP36L2, CD37, HSP90AA1, and TMSB4X.

The identification criteria are as follows: these transcription factors are widely and highly expressed in hematopoietic progenitor cell subpopulations, with broad and high expression in both progenitor and mature cell populations, showing no obvious specificity. These genes, along with the first category of transcription factors and genes, collectively participate in the fate determination and regulation of hematopoietic stem cells, including their function and differentiation. Cell membrane surface expression genes can be used for the identification and sorting of hematopoietic stem cells. The expression profiles of these transcription factors and key genes are shown in FIG. 7.

### Second Category: Key Transcription Factors for Megakaryocyte-Erythroid Progenitor Cells

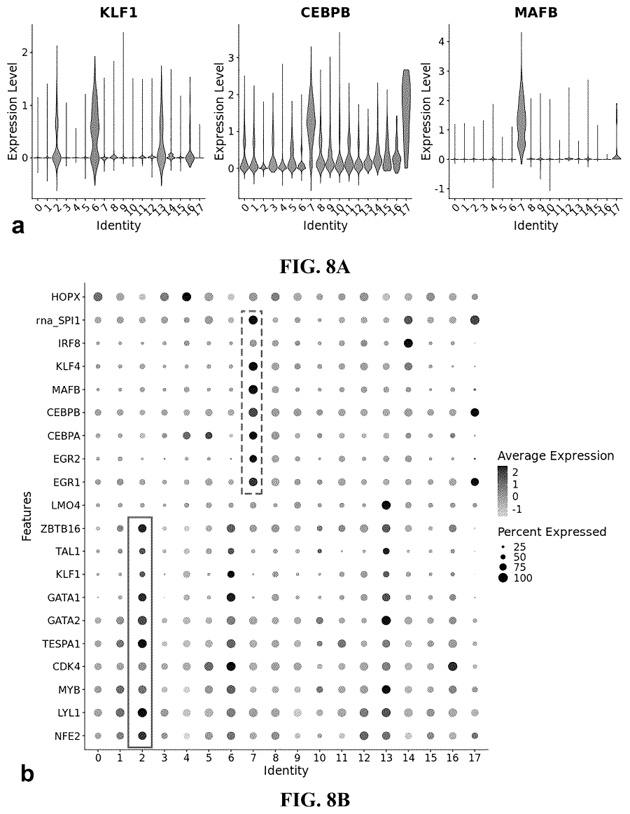

These include early megakaryocyte-erythroid progenitor (GAPs, NMPs) transcription factors (NFE2, LYL1, MYB, CDK4, TESPA1, and GATA2), megakaryocyte-erythroid and mast cell lineage transcription factors (GATA1 (ENSG00000102145), KLF1, TAL1, ZBTB16, LMO4) (FIG. 8B, solid-line frame); Neutrophil-Monocyte progenitor (NMP) transcription factors (SPI1, KLF4), including macrophage progenitor transcription factors (EGR1, EGR2, CEBPA, CEBPB, MAFB) and dendritic progenitor transcription factors (IRF8) (FIG. 8B, dashed-line frame). Their main function is to control the differentiation direction and pathway of GAPs and NMPs, and they are fate-determining factors for progenitor cells. The expression profiles are shown in FIGS. 8A and 8B, where the circle size represents the expression proportion, and the color intensity reflects expression abundance.

### Third Category: Key Transcription Factors for Lymphoid Hematopoietic Progenitor Cells

These include JUN/FOS, RUNX2 (ENSG00000124813), HOXA9, TCF4, DDIT4, HOPX, KLF10, HOXA3, and TSC22D1, which primarily control the differentiation direction and pathway of lymphoid progenitor cells and are fate-determining factors for lymphoid progenitor cells.

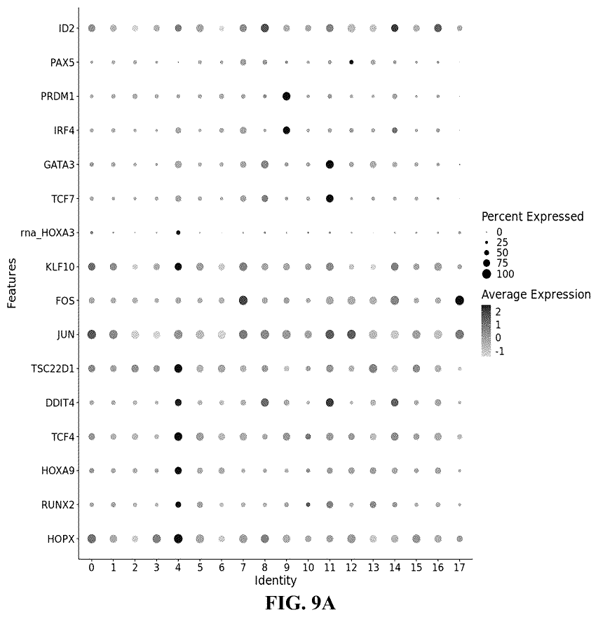

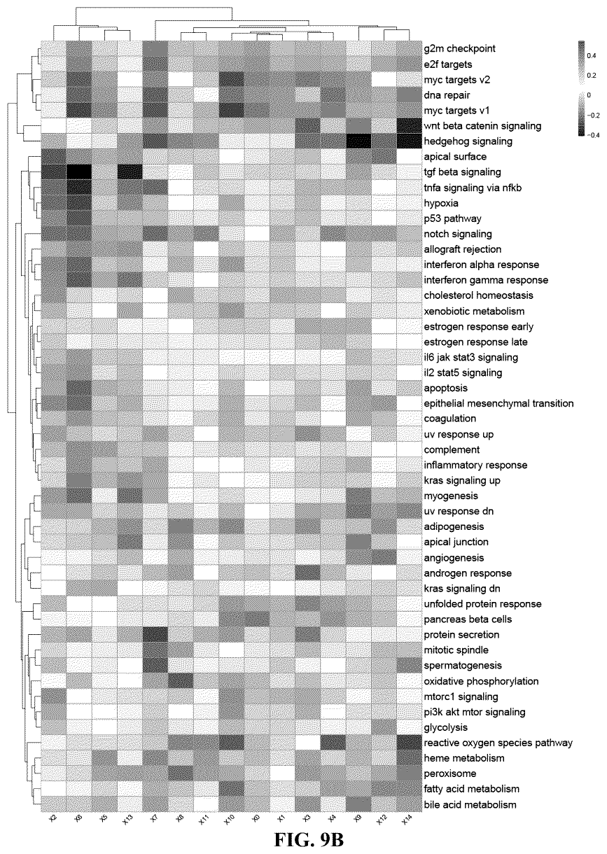

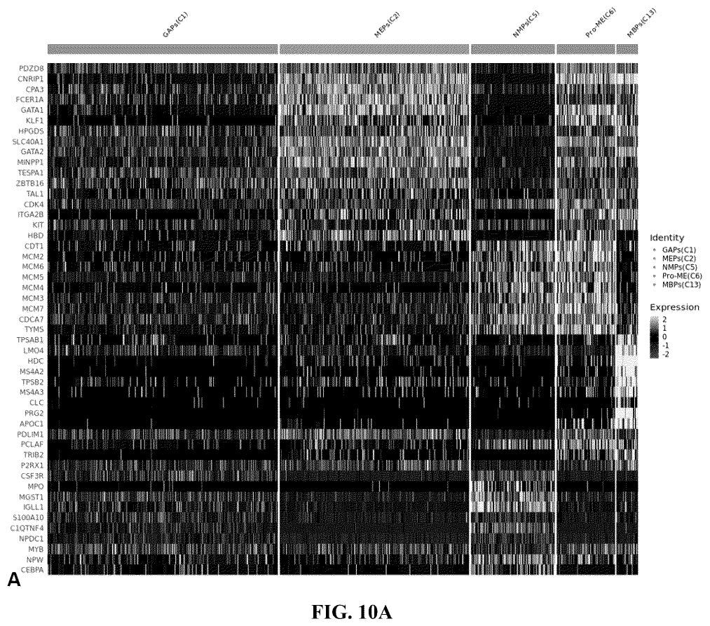

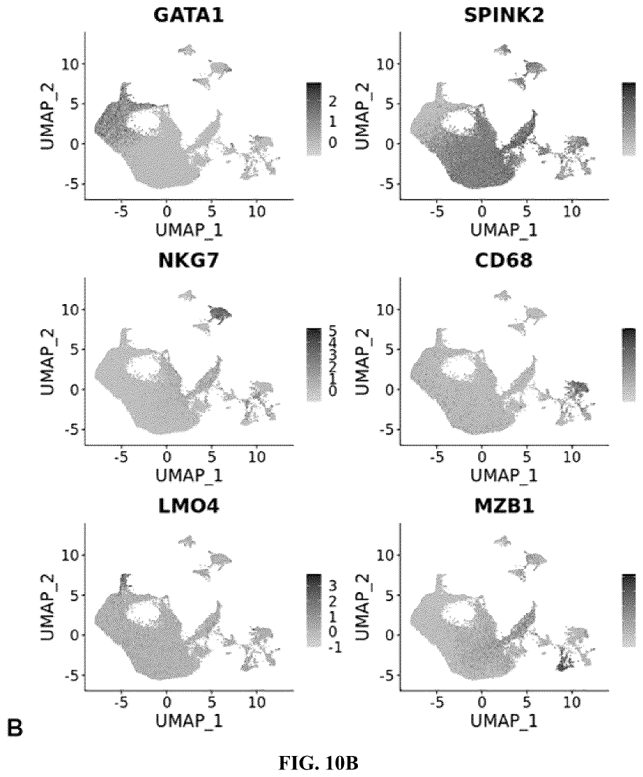

The expression profiles of key transcription factors for lymphoid progenitor cells were shown in FIG. 9, where the key transcription factors for T lymphoid progenitors are GATA3, TCF7, and BCL11B; for NK progenitors, they are HOPX, DDIT4, ID2, and TBX21; for B lymphoid progenitors, the key transcription factor is PAX5; for plasma progenitors, it is IRF4 and PRDM1. In FIG. 9, these genes were highly expressed in the differentiation trajectory of lymphoid progenitor cell subpopulation 4, but exhibit low expression in the myeloid differentiation trajectory of progenitor cell groups 1 and 2. The temporal and spatial expression features of these transcription factors (FIG. 10B) include the time points and stages of their expression during differentiation in each lineage progenitor, specific subpopulations, as well as the expression profile features and dynamic changes in expression due to activation or inhibition.

Based on the identified lineage and gene expression profiles of hematopoietic progenitor cells, Gene Set Variation Analysis (GSVA) was used to analyze the molecular expression networks of different lineage subpopulations. Gene sets and key regulatory signaling networks for different progenitor cell subpopulations were obtained (FIG. 9B). The results show that mTORC1 signaling is suppressed during megakaryocyte-erythroid progenitor differentiation, with its related inhibitory gene DEPTOR highly expressed in the megakaryocyte-erythroid progenitor lineage. In other embodiments, mTORC1 signaling inhibition through rapamycin treatment enhances differentiation towards the megakaryocyte-erythroid lineage (a well-established pathway). Similarly, the signaling expression profiles in this invention show that TGF-β is enriched in quiescent progenitor cells, and also demonstrate the expression or suppression of signaling pathways such as Notch, KRAS, and TNF-α in various progenitor cell lineages. In previously reported embodiments, inhibition of key signaling pathways, such as EGFR, KRAS, etc., has been used for drug development and target identification, therefore applied in disease treatment and control, with many successful cases reported in cancer and other diseases treatment.

By identifying the fate-determining factors that regulate or drive progenitor cell differentiation, and using well-known techniques (such as gene knockout or drug intervention), effective control of cell fate can be achieved (R. Drissen, 2016, Nat Immunol). The feasibility and effectiveness of this approach have been extensively validated (e.g., transcription factor reprogramming of stem cells and growth factor-induced stem cell culture in vitro). The main obstacle to its application lies in identifying the key fate-determining factors. Therefore, based on the preceding examples of the invention, which accurately identify various progenitor cell types, clearly define differentiation pathways, directions, and nodes, and identify fate-determining genes for various progenitors, the invention can achieve intervention and regulation of differentiation in diverse cell lineages by the intervention of key fate-determining functional genes (e.g., reprogramming transcription factors, see Example 8) or molecular signaling networks (e.g., in vitro induction via growth factor receptor activation, see Example 6) within progenitor subpopulations in combination with gene expression profile characteristics of cell subpopulations.

Specifically, at distinct differentiation stages or directions, diverse agents (e.g., peptides, small molecule drugs, receptor molecules) and techniques (e.g., gene editing, gene transfection) can be applied to regulate the activation or inhibition of transcription factors and receptor genes at defined time points or stage-specific locations. This enables precise control over the differentiation direction and developmental stages of progenitor cells.

### IV. Identification of Peripheral Blood HPC Lineage-Specific Marker Genes and Fate Determining Genes

For the various mature cell subpopulations in the hematopoietic system, many markers are known and widely recognized for identifying mature cell types, such as macrophage and monocyte markers CD68 and CD86, NK cell marker NKG7, lymphoid cell markers MME, IGHM, CCR7, key transcription factors for myeloid cells like GATA2 and KIT, erythroid marker genes HBD and KLF1, and neutrophil marker gene MPO, etc. Among the key marker genes identified for progenitor cells in this embodiment, the aforementioned known and widely recognized marker genes are included (consistent evidence), and they correspond to different stages of the same type of cell subpopulations (progenitor stage vs. mature stage). This further corroborates the accuracy and reliability of the progenitor cell subpopulations defined and identified in the previous examples of this embodiment. The novelty of the present invention lies in the application of transcription factors for precise cell type identification and lineage classification. The marker genes and fate-determining genes described in this invention are not clearly differentiated; typically, marker genes are more likely to refer to genes used for cell classification, while fate-determining genes are more likely to refer to transcription factors, growth factor receptors, and other such genes.

This embodiment identified new marker genes and transcription factors for various progenitor cell subpopulations, as shown in Table 1, to accurately define and identify progenitor cell subpopulations across different lineages, differentiation stages and directions. The criteria for screening differentially expressed marker genes and transcription factors are as follows:

Genes that are specifically expressed or highly expressed in a certain differentiation direction, with statistically significant expression levels, including the key genes and transcription factors identified above.

Genes with positive expression across all subpopulations cannot be used to distinguish subpopulations. Even if the expression level is high and the difference is statistically significant, these genes are excluded.

Genes like IGLV, IGKV, and HLA, which are known to be specific to lymphoid cell lineages, are excluded.

Differential genes that are highly expressed or specifically expressed when comparing GAPs (C2) subpopulations with lymphoid subpopulations, as well as the differential analysis of each subpopulation alone.

Genes that are widely expressed in the PBMC control group are excluded.

The expression profile characteristics of the marker genes and transcription factors include whether to express, expression levels, spatial distribution, temporal dynamics, and differentiation stage and direction linked progenitor-cell categories. (FIGS. 4-11). The characteristics of the newly identified marker genes and transcription factors for each progenitor cell subpopulation are as follows:

The marker genes provided in Table 1 can clearly distinguish progenitor cell subpopulations. Individual or combinations of multiple genes can more effectively differentiate and identify each progenitor cell population, including genes with negative or very low expression, which are used to distinguish different lineages or differentiation stages.

The expression characteristics of gene combinations as markers distinctly identify progenitor cell characteristics at different stages. For example, GATA2 alone does not effectively distinguish different stages of the megakaryocyte-erythroid cell subpopulations, but combining GATA1 with GATA2 enables differentiation stage identification of megakaryocyte-erythroid progenitor cells, achieving effective distinction of megakaryocyte-erythroid progenitors at different differentiation stages.

The expression of multiple known and widely recognized mature cell type markers is consistent with the identification results of the progenitor cell types in this embodiment, further validating the reliability of the identified markers. The technical novelty of this embodiment lies in including one or more newly identified marker genes and using multiple transcription factors to explicitly define and identify progenitor-cell subpopulations at specific differentiation stages and lineage commitment pathways.

Based on the gene features described, selecting membrane protein genes with lineage-specific expression enables the sorting, enrichment, or capture of target cell subpopulations from different lineages, achieving the enrichment and application of hematopoietic cell subpopulations with high purity. Traditional sorting typically relies on markers like CD123, CD135, CD45RA, CD34, CD38, and CD10, and the limitations of these traditional markers have already been discussed in the expression profiles (FIG. 4). Based on the specificity, directionality, and developmental stage-associated features of the gene expression profile in this invention, the method enables the effective combination of target cell sorting markers, thereby achieving precise capture, sorting, and subsequent enrichment of cells. For instance, the megakaryocyte-erythroid lineage can be sorted using a combination of (CD34+FLT3−) along with one or more membrane protein markers that are specifically and positively expressed (CNRIP1, CPA3, FCER1A, SLC40A1, CSF2RB, etc.) (FIG. 4). Adding MS4A2 or MS4A3 enables specific capture, identification, and distinction of the MBPs subpopulation within the megakaryocyte-erythroid lineage (FIG. 5B). Similarly, CD34+ MME+(or LTB+SLC2A5+) can be used to sort common lymphoid progenitor cells (CLPs), CD34+ CSF1R+ to sort macrophage progenitors, and CD34+ FCER2+ to specifically enrich the B cell progenitor subpopulation. All other lineages can also be sorted and enriched according to these principles (FIG. 4, Table 1).

Based on the correlations between the expression profile features and the progenitor cell characteristics, the marker gene expression profile enables the identification of differentiation stages, directions, and pathways for progenitor cell lineages. The marker combination comprises antibodies or peptides targeting positive/negative expression markers, which can be used to sort progenitor cell lineages by conventional flow cytometry, magnetic bead-based separation, or the rosette assay, these methods being well-established in the field.

Thus, based on the preceding embodiments of this invention, each progenitor cell type has been accurately identified, with clear differentiation paths, directions, nodes, and stages. The identification of marker genes and expression profiles for each progenitor cell subpopulation enables the use of one or more membrane protein genes from the marker genes of each lineage cell subpopulation (as shown in Table 1) to create antibody, peptide, or recombinant protein markers for distinguishing, identifying, sorting, and enriching progenitor cell subpopulations at different differentiation stages, directions, or nodes, achieving differentiation, identification, sorting, and enrichment of single or single-lineage progenitor cell subpopulations with high-purity (FIGS. 4, 5B, 10B). This also includes methods for detecting the quantity, state, and expression profiles of HPC populations or subpopulations across various lineages. This can be applied to isolate different types of progenitor cells in blood under various physiological or pathological conditions, including but not limited to blood diseases, immune conditions, or infection states. Based on highly accurate, specific expression profiles of various progenitor cell types, specific antibody combinations can be used for labeling and separating progenitor cells, addressing the challenges in subsequent research and applications of rare progenitor cells.

HSC transplantation is currently one of the effective treatments for leukemia, but remains limited by immune rejection. Therefore, identifying HSC differentiation stages and pathways, and selecting the target cell population for transplantation for specific tumor types, can effectively avoid transplanting off-target stem cells and reduce immune reactions or rejections.

TABLE 1
Key marker genes and transcription factors defining progenitor subpopulations

| Redefined | | | |
|---|---|---|---|
| progenitor | | | Negative or low |
| cell types | Marker genes | Transcription factors | expression genes |
| CLPs | SPINK2, HOPX, HOXA9, | HOPX, DDIT4, HOXA9, | CNRIP1, FCER1A, |
| (C4) | RUNX2, LTB, IGHM, DNTT, | RUNX2 | GATA1, |
| | PRSS2, SLC2A5, MME, CCR7, | | S100A10 |
| | NKG7, LST1, BASP1, CD79A, | | |
| | MZB1, FLT3, SPON1 | | |
| Pro-NK | GNLY, NKG7, CD247, CCL5, | DDIT4, HOPX, ID2, | IL7R, GATA3 |
| (C8) | FCGR3A, PRF1, GZMA, GZMB, | TBX21 | |
| | KLRD1, KLRB1, KLRF1, | | |
| | CD3E, CD7, HOPX, IL2RB, | | |
| | TBX21, ID2 | | |
| Pro-T | TCF7, IL7R, GATA3, KLRB1, | TCF7, GATA3, DDIT4, | GNLY, FCGR3A, |
| (C11) | CD3E, CD3D, CD7, CD247, | BCL11B | GZMA |
| | LTB, BCL11B, DDIT4 | | |
| Pro-B1 | CD19, | PAX5 | CD27 |
| (C12) | MS4A1, FCER2, CD79A, | | |
| | CD79B, IGHM, LTB, IGKC, | | |
| | PAX5, VPREB3, CD22, CD24, | | |
| | FCRLA | | |
| Pro-B2/ | CD27, CD38, IGKC, IGHA1, | PRDM1, IRF4 | MS4A1, FCER2 |
| Pro-plasma | SLAMF7, CD79A, CD79B, | | |
| (C9) | PRDM1, IRF4, JCHAIN, IFI30 | | |
| NMPs | CSF3R, MPO, MGST1, IGLL1, | MYB, CDK4, CEBPA | GATA2, |
| (C5) | S100A10, C1QTNF4, NPDC1, | | SLC40A1, |
| | MYB, CDK4, CDCA7, CEBPA, | | CNRIP1, LTB |
| | NPW | | |
| Lineages | CNRIP1, CPA3, FCER1A, | GATA1, ZBTB16, | FLT3, SPINK2, |
| (C2, C6, | GATA1, KLF1, HPGDS, | TAL1, | HOPX, |
| C13) | SLC40A1, GATA2, | CDK4, TESPA1, KLF1 | C1QTNF4, CSF3R |
| | ZBTB16, TAL1, TESPA1, | | |
| | MYB, CDK4, ITGA2B, | | |
| | MINPP1, PDZD8, KIT, | | |
| | CSF2RB | | |
| GAPs (C1) | GATA2, NFE2, LYL1, MYB, | GATA2, NFE2, LYL1, | GATA1, KLF1 |
| | SLC40A1, TESPA1, CSF3R | MYB | |
| MEPs (C2) | GATA2, NFE2, LYL1, MYB, | GATA2, NFE2, LYL1, | CSF3R |
| | GATA1, KLF1, CSF2RB, | MYB, GATA1, KLF1, | |
| | SLC40A1 | ZBTB16, TAL1, CDK4, | |
| | | TESPA1 | |
| Pro-ME | HBD, CDT1, MCM2, MCM6, | CDK4 | FLT3, SPINK2, |
| (C6) | MCM5, MCM4, MCM3, MCM7, | | HOPX, C1QTNF4, |
| | CDCA7, CDK4, TYMS | | CSF3R, |
| | | | MS4A2, MS4A3 |
| MBPs | TPSAB1, LMO4, HDC, MS4A2, | LMO4, CDK4, MITF | HBD |
| (C13) | TPSB2, MS4A3, KIT, PRG2, | | |
| | CLC, MCM2-MCM7, APOC1, | | |
| | MITF, TRIB2 | | |
| Pro-Eosinophil | CLC, HDC, RFLNB, MEIS1, | ETV6 | MS4A2, TPSB2, |
| (C10) | ETV6 | | MS4A3 |
| Pro-Monocytes1/ | EGR1, SPI1, KLF4, CEBPB, | SPI1, KLF4, CEBPB, | CLEC9A, THBD, |
| Pro-Mac | FCGR3A, CSF1R, CD68, CD86, | EGR1, EGR2, CEBPA, | IRF8 |
| (C7, | ITGAX, FCGR2A, LYZ, LST1, | MAFB, BCL6, NR4A1 | |
| Macrophages) | EGR2, CEBPA, MAFB, TNF, | | |
| | BCL6, LILRB2, CD4, CD33, | | |
| | FCGR2A, IFI30, S100A9, | | |
| | NR4A1, HMOX1, C5AR1, CD83 | | |
| Pro-Monocytes2/ | CLEC9A, ANPEP, THBD, | SPI1, KLF4, IRF8, | FCGR3A, CSF1R, |
| Pro-DCs | IRF8, KLF4, CD68, CD86, | DDIT4, BCL6 | MAFB |
| (C14, DCs) | ITGAX, LYZ, SPI1, LST1, | | |
| | DDIT4, SLAMF7, BCL6, | | |
| | BASP1, CD4, CD33, IFI30, | | |
| | CD83 | | |

Note: The reference database for gene name annotations: refdata-gex-GRCh38-2020-A, version 32 (Ensembl 98).

The marker genes and their expression profile features defined in Table 1 (directionality, developmental stage-associated features, etc.) enable precise identification and definition of progenitor cell lineage subpopulations. The expression profile data (matrix) of this invention includes expression profile features for many genes from various cell subpopulations. While certain embodiments exemplified in Table 1 do not exhaustively enumerate all candidate genes, any gene functionally equivalent to the disclosed expression biomarkers—including but not limited to lineage-restricted or differentiation-stage-dependent expression patterns—falls within the scope defined here. The protection scope of this invention explicitly encompasses said genes. The identified cell lineages and their characteristic biomarkers shall not be construed as limited to the exemplars enumerated in Table 1.

### Expression Profile Verification of Fate Determining Genes and Marker Genes for Lymphoid and Myeloid Progenitor Cells

Various progenitor cell lineages within hematopoietic progenitor cells are derived from the same population of HSCs. The differentiation and maturation of HSCs are not solely determined by transcription factors. During the differentiation of HSCs, key genes that determine differentiation into lymphoid progenitor cells and myeloid hematopoietic progenitor cells, in addition to the transcription factors identified above, the expression or activation of other genes also play a critical role in the differentiation direction and fate determination of cell lineages.

Based on the aforementioned steps II and III of this example, the initial differentiation stage of hematopoietic progenitor cells shows three differentiation directions simultaneously. Step III identifies the fate-determining transcription factors for three types of hematopoietic progenitor cell lineages. Step IV involves the expression profile analysis of various progenitor cell subpopulations, which redefines and identifies key marker genes for these subpopulations (Table 1). Further, differential analysis of gene expression profiles is performed to validate the expression profiles of three major differentiation directions (lineages): lymphoid (C4), megakaryocyte-erythroid (C1 and C2), and neutrophil-monocyte progenitor cells (C5), and to present genes with different expression features and their significance. Here, the specific marker genes for each lineage and progenitor cell subpopulation in the three differentiation directions were identified, providing a list of fate-determining genes for lineage differentiation and commitment. The accuracy and reliability of the redefined hematopoietic progenitor cells are validated.

Validation Results of Megakaryocyte-Erythroid Progenitor Cell Gene Expression Heatmap (FIG. 10A) and Characteristics:

18 genes, including SLC40A1, CNRIP1, CPA3, GATA2, FCER1A, MINPP1, GATA1, KLF1, ZBTB16, PDZD8, TPSAB1, HPGDS, TAL1, CDK4, ITGA2B, MINPP1, KIT, and CSF2RB, are co-expressed and significantly up-regulated in megakaryocyte-erythroid progenitor cells (C2, C6, C13 subpopulations), showing lineage-specific features.

Genes such as GATA2, NFE2, LYL1, MYB, GATA1, KLF1, CSF2RB, and SLC40A1 are expressed in megakaryocyte-erythroid progenitor subpopulation MEPs (C2 subpopulation) and do not express the CSF3R gene. Eleven genes, including HBD, CDT1, MCM2, MCM6, MCM5, MCM4, MCM3, MCM7, CDCA7, CDK4, and TYMS, are differentially expressed in megakaryocyte-erythroid precursor progenitor cells Pro-ME (C6 subpopulation). Eighteen genes, including TPSAB1, LMO4, HDC, MS4A2, TPSB2, MS4A3, KIT, PRG2, CLC, MCM2, MCM6, MCM5, MCM4, MCM3, MCM7, APOC1, MITF, and TRIB2, show significant up-regulation in mast cell and basophil progenitor cells (C13 subpopulation), demonstrating lineage-specific characteristics.

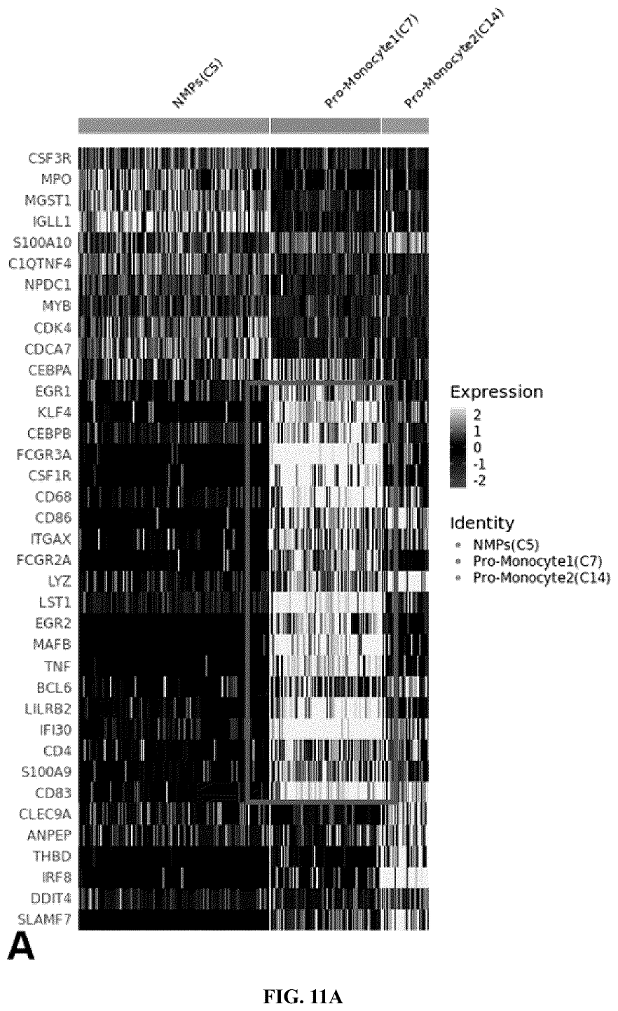

### Validation Results for Monocyte-Granulocyte Progenitor Cell Lineage Marker Genes (FIG. 11A):

IC1QTNF4, NPDC1, MPO, CSF3R, MGST1, IGLL1, NPW, S100A10, CDCA7, CEBPA, MYB, CDK4, and other genes show up-regulated expression in the neutrophil-monocyte progenitor cell differentiation direction. Among these, GATA1, KLF1, MPO, CSF3R, and GATA2 are previously established fate determining genes of myeloid lineage commitment. Distinctly from known, the identified expression profile characteristics correspond to lineage-specific differentiation stages. For example, GATA1 expression is restricted to late-stage of megakaryocyte-erythroid progenitor cells, while GATA2 does not participate in neutrophil differentiation and serves as a critical determinant governing progenitor cell lineage bifurcation into neutrophils or megakaryocyte-erythroid progenitors.

Macrophage Progenitor Cells (C7, Macrophages): EGR1, SPI1, KLF4, CEBPB, FCGR3A, CSF1R, CD68, CD86, ITGAX, FCGR2A, LYZ, LST1, EGR2, CEBPA, MAFB, TNF, BCL6, LILRB2, CD4, CD33, FCGR2A, IFI30, S100A9, NR4A1, HMOX1, C5AR1, CD83.

Dendritic Progenitor Cells (C14, DCs): CLEC9A, ANPEP, THBD, IRF8, KLF4, CD68, CD86, ITGAX, LYZ, SPI1, LST1, DDIT4, SLAMF7, BCL6, BASP1, CD4, CD33, IFI30, CD83.

Characteristics of the above expression profiles:

Lineage-Specific Expressed Genes: These genes can identify the differentiation direction of progenitor cell lineages. For example, GATA1 and TAL1 are specifically expressed in the differentiation direction of megakaryocyte-erythroid lineage (C2, C6, C13 subpopulations), and based on the expression of one or more genes, the differentiation direction towards megakaryocyte-erythroid progenitors can be clearly determined (FIG. 10B). Moreover, these lineage-specific expression features can also be used for lineage tracking.

Progenitor Cell Differentiation Stage Identification: For instance, LMO4, TPSB2, MS4A3, etc., are expressed only in mast cell and basophil progenitors. Therefore, if the expression of such genes increases significantly during progenitor cell differentiation, it indicates that the differentiation process has entered the mast cell and basophil progenitor stage (FIG. 10B).

Precise Control of Differentiation: LMO4 and MITF are key transcription factors for mast cell differentiation. By using gene knockout or small-molecule interventions to inhibit their activity, the differentiation of progenitor cells towards erythrocytes can be effectively controlled, reducing or inhibiting the formation of mast cells and controlling the precise differentiation pathway of the cells. Similarly, CSF3R and KIT are key growth factor receptors for progenitor cell differentiation. Selective activation of these receptors can control the induced differentiation of progenitor cells.

Coordinated Regulation of Lineage Commitment: The marker genes are jointly involved in cell lineage differentiation and fate determination, including expression activation, up-regulation or suppression, not being isolated events. For example, up-regulation of GATA2 in combination with suppression of the CSF3R signaling pathway control differentiation of progenitor cells toward megakaryocyte-erythroid progenitors.

Continuous Lineage Commitment in MEPs: Seventeen MEP-specific marker genes (including GATA2) display a gradually up-regulated expression profile during differentiation (FIG. 4), defining the lineage formation pattern of MEPs a continuous lineage commitment mode. This aligns with the physiological characteristic of human erythropoiesis, wherein erythrocytes undergo perpetual, high-volume renewal within the hematopoietic system.

Common Lymphoid (C4) and Lymphoid Progenitor Cell Subpopulation Marker Gene Expression Profile and Characteristics Verification Results (FIG. 11B):

Common Lymphoid Progenitor (CLP) Genes: LTB, SPINK2, HOPX, PRSS2, IGHM, DNTT, SLC2A5, MZB1, LST1, HOXA9, NKG7, BASP1, RUNX2, MME, CCR7, CD79A, SPON1.

NK Progenitor Cell Marker Genes: GNLY, NKG7, CD247, CCL5, FCGR3A, PRF1, GZMA, GZMB, KLRD1, KLRB1, KLRF1, CD3E, CD7, HOPX, IL2RB, ID2; showing lineage-specific characteristics.

T Progenitor Cell Marker Genes: TCF7, IL7R, GATA3, KLRB1, CD3E, CD3D, CD7, CD247, LTB, DDIT4; showing lineage-specific characteristics.

B Progenitor Cell Marker Genes: CD19, MS4A1, FCER2, CD79A, CD79B, IGHM, LTB, IGKC, PAX5, VPREB3, CD22, CD24, FCRLA; showing lineage-specific characteristics.

Plasma Progenitor Cell Marker Genes: CD27, CD38, IGKC, IGHA1, SLAMF7, CD79A, CD79B, PRDM1, IRF4, JCHAIN, IFI30; showing lineage-specific characteristics.

The characteristics of expression profiles of the above lymphoid progenitor cell subpopulations are as follows:

Lineage-Specific Expressed Genes: These genes can identify the differentiation direction of progenitor cell lineages. For example, SPINK2 is expressed only in lymphoid progenitor cells (FIG. 10B), and genes such as NKG7 in NK cells, GATA3 in T cells, MS4A1, CD79A in B cells, and CD27 in plasma cells show specific positive expression, indicating the differentiation direction and type of progenitor cells. These gene expression patterns can also be used for lineage tracking.

Progenitor Cell Differentiation Stage Identification: In B lymphoid progenitor cells, both B progenitors and plasma progenitors exist, and their molecular expression profiles are quite similar. The co-expression of CD79A and CD27 indicates that the differentiation direction of the progenitor cells is towards B progenitors, and the differentiation has entered the plasma progenitor stage.

Precise Control of Differentiation: PAX5 in B progenitor cells and GATA3 in T progenitor cells are key transcription factors that determine the fate of differentiation. During progenitor cell differentiation, gene knockout or small molecule interventions to inhibit their activity can effectively reduce or inhibit the formation of B or T cells, achieving precise control of the differentiation pathway. Similarly, IL2RB is a key growth factor (IL15) receptor for NK progenitor cell differentiation. Selective activation of the IL2RB receptor can control the induced differentiation of progenitor cells into NK cells.

Coordinated Regulation of Lineage Commitment: The aforementioned lineage-specific marker genes are jointly involved in cell lineage differentiation and fate determination, including expression activation, up-regulation or suppression, not being isolated events.

Stepwise Differentiation in Lymphoid Lineages: Unlike the expression profile characteristics of megakaryocyte-erythroid progenitors, the gene expression of lymphocytes mostly follows a stepwise activation pattern (FIG. 4). For instance, many marker genes for T cells, B cells, and NK cells are not expressed in the common lymphoid progenitor (CLP) subpopulation. The stepwise lineage commitment pattern aligns with the physiological and immune response characteristics of the human immune system, where various immune cells undergo significant proliferation only upon external stimuli (viral infection or pathogenic stimuli) occur.

In this embodiment, the expression profiles of marker genes and transcription factors in hematopoietic stem cells and progenitor cell subpopulations validated the characteristics of progenitor cells differentiating into three main directions (lineages). The key differential marker genes and transcription factors serve as fate-determining factors for the three different differentiation directions (lineages), including transcription factors, growth factor receptors, and the marker genes involved in fate determination (Table 1). Differentially expressed genes identified in differential expression profiles function as key regulatory determinants that dynamically modulate stem/progenitor cell differentiation tendencies and directions across distinct developmental stages during HSC differentiation and lineage tree formation. The identified expression profiles of fate-determining genes and marker genes encompass not only gene transcriptional signatures, but also differentiation stage and direction information during progenitor cell differentiation, with stage-resolved signatures, and lineage-resolved signatures (FIG. 10B). Specifically, lineage-resolved signatures are defined by genes (e.g., SLC2A5, KLF1, GATA1, HOXA9, and SPINK2) that exhibit expression exclusively along distinct differentiation trajectories of particular lineages (FIG. 10B). Stage-resolved signatures are characterized by the emergence of novel marker gene expressions at defined differentiation stages of hematopoietic stem cells. The expression trends (activation dynamics) and spatial distributions of these genes are stage-specific. Combining multiple marker genes enables precise identification of the current differentiation stage in progenitor cells, such as those in the megakaryocyte-erythroid lineage.

### Example 4: Identification of Human Hematopoietic Progenitor Cell Subpopulations and Lineages and Validation of Redefined Marker Genes

Hematopoietic progenitor cells ultimately differentiate into various mature cell lineages. Marker genes with stem cell characteristics, such as CD34, CD133, and CD117 (KIT), typically disappear during the differentiation and maturation process. However, lineage-specific marker genes, particularly those differentiation stage-resolved of progenitor cells, are still frequently expressed in mature cells of the same lineage post-maturation. For example, the marker genes CD19 and MS4A1 (CD20) for B cells, CD68 and CD86 for monocyte-macrophages, GZMA and KLRD1 (CD94) for NK cells, CD3D and CD3E for T cells, HBD for erythrocytes, and MPO for neutrophils. The expression profiles of these canonical mature cell markers significantly overlap with those in identified and redefined HPC subpopulations characterized in Example 3. (FIGS. 4, 10A-10B, and 11A-11B). The majority of cell surface antigens utilized to redefine progenitor cell subpopulations throughout Examples 3 align with established definition criteria for these populations (Table 1), thereby supporting their validity without necessitating additional experimental confirmation. This all demonstrates the reliability and accuracy of the methods for progenitor cell identification and definition in the present invention.

The innovation in the example 3 lies in the use of transcription factors to redefine progenitor cell subpopulations. The expression profile characteristics have been shown to be highly consistent with the properties of the redefined cell subpopulations (FIGS. 4, 8A-8B, and 9A-9B). Established lineage-specific transcription factors, such as GATA1, MAFB, PAX5, and GATA3, were expressed exclusively in their corresponding progenitor cell subpopulations. Further, additional transcription factors and marker genes disclosed in this invention were validated to further support the accuracy and reliability of the expression profile data and the redefined scheme.

### 1. Fluorescent Quantitative PCR Validation of Lineage-Specific Marker Genes

i. Acquisition of Different Cell Components and Tumor Cell Lines
A1. Isolation of T Cells, B Cells, NK Cells, and Monocyte-Granulocyte Cells from Peripheral Blood Using Immunomagnetic Beads (Miltenyi Biotec)

Peripheral blood mononuclear cells (PBMCs) were isolated using Ficoll (GE, Cytiva) density gradient centrifugation.

Magnetic beads were used for cell labeling and separation:
a) CD19-positive B cells were isolated using magnetic separation.
b) CD3-positive T cells were isolated.
c) CD56-positive NK cells were isolated.

### A2. Acquisition of Hematologic Tumor Cell Lines

The following hematologic tumor cell lines were obtained from cas9x Biotech(China) and cultured in corresponding standard medium according to ATCC standards:
K562 (myeloid lineage)
THP-1 (monocytic lineage)
Jurkat (lymphoid lineage)
MEG01 (megakaryocytic lineage)
NK92 (NK cell lineage)

Despite exhibiting divergent transcriptional profiles compared to normal cells, these tumor cell lines retain lineage-specific markers, which enables the validation of key transcription factors and gene expression patterns.

B. RNA Extraction and cDNA Synthesis

The magnetically separated cell components and cultured tumor cell lines were lysed with Trizol.

RNA was extracted and reverse transcribed into cDNA.

C. Fluorescent Quantitative PCR (qPCR) for Gene Expression Analysis (TAKARA)

C1. PCR Reaction System Setup (Table 2)

A 20-μL PCR reaction system was prepared in RNase/DNase-free EP tubes using reagents listed in Table 2. Primer sequences for each gene were referenced from the PrimerBank database (https://pga.mgh.harvard.edu/primerbank/links.html).

TABLE 2
PCR Reaction System

| Reagent | Amount | | Final Concentration |
|---|---|---|---|
| TB Green Premix Ex Taq II (2X) | 10 | μL | 1X |
| PCR Forward Primer (10 μM) | 0.8 | μL | 0.4 μM |
| PCR Reverse Primer (10 μM) | 0.8 | μL | 0.4 μM |
| ROX Reference Dye or Dye II (50X) | 0.4 | μL | 1X |
| cDNA | 2 | μL | / |
| Sterile Water | 6 | μL | / |
| Total Volume | 20 | μL | / |

C2. Sample and primer names were configured and SYBR Green method was used to detect gene expression levels.

C3. The configured system was placed into a QPCR instrument, parameters were configured on the ViiA 7 RUO Software.

C4. Two-step PCR conditions were used as shown in Table 3.

TABLE 3
PCR conditions

| Step | Temperature | | Time | | Cycles | Purpose |
|---|---|---|---|---|---|---|
| Stage 1 | 94° | C. | 30 | s | / | Pre-denaturation |
| Stage 2 | 94° | C. | 15 | s | 40 | PCR Amplification |
| | 60° | C. | 30 | s | | |
| Stage 3 | 4° | C. | 20 | min | / | |

C5. Gene expression Ct values were recorded and expression differences were calculated after the PCR reaction.

D. Validation of Fate-Determining Transcription Factors and Redefined Marker Genes in Human Hematopoietic Progenitor Subpopulations and Lineages

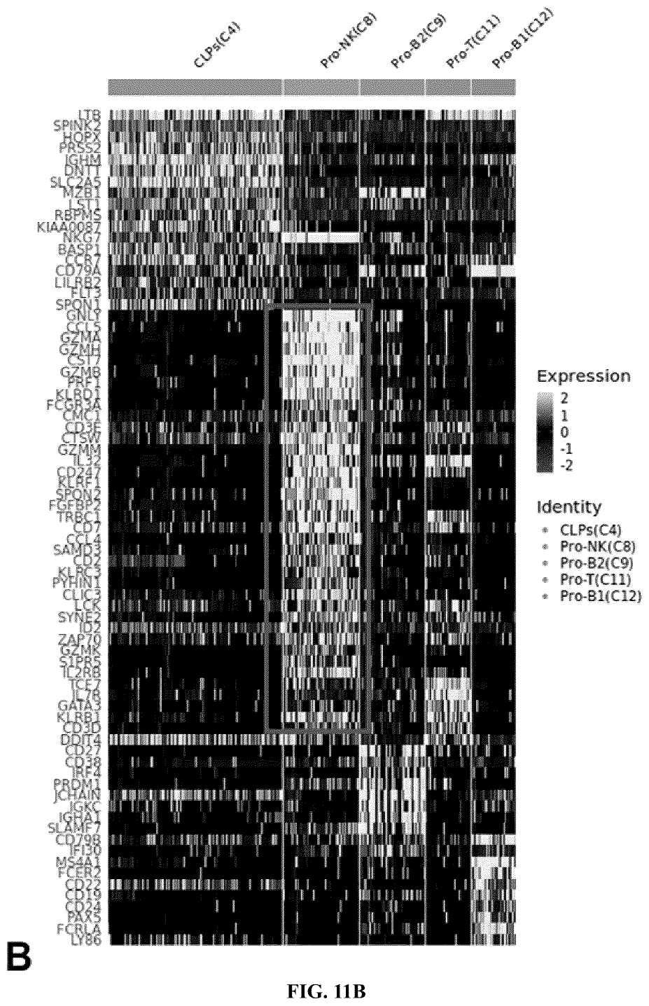

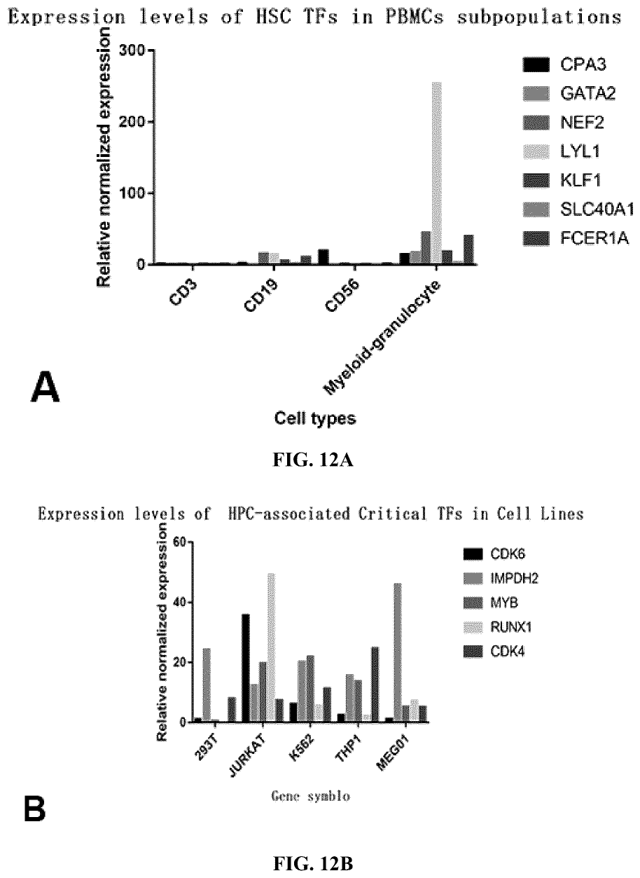

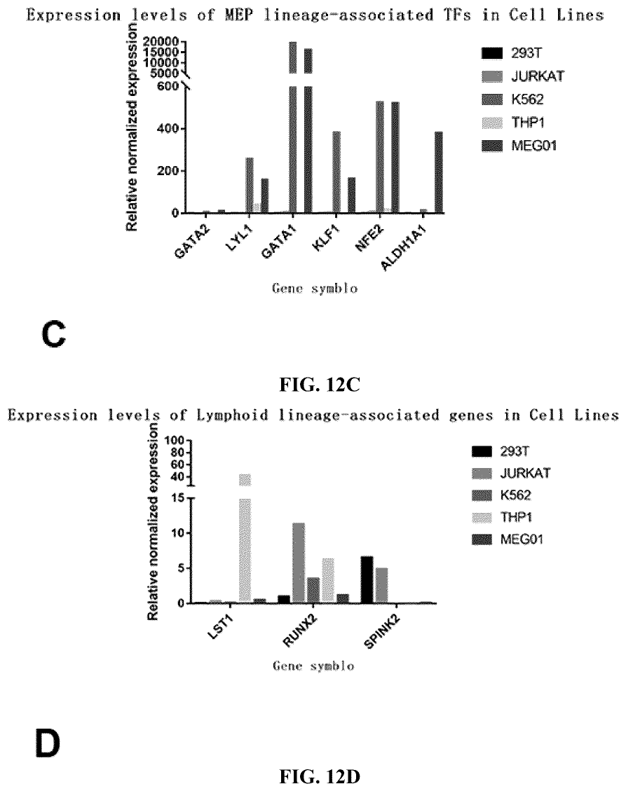

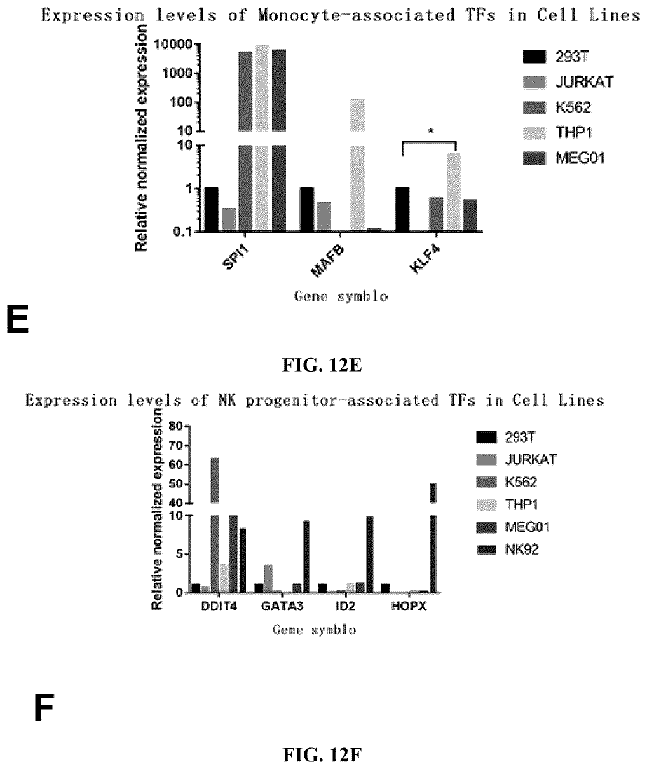

Quantitative PCR results (FIGS. 12A-12F) show that CYTL1 and EGFL7 have very low or no expression in mature hematopoietic cells, confirming them as hematopoietic stem cell (HSC) markers.

The expression of HOPX and SPINK2 in PBMCs subpopulations showed no significant difference, due to the limited sensitivity of qPCR-based profiling and unresolved transcriptional heterogeneity in magnetically isolated subpopulations. (FIG. 12A).

The third-party single-cell transcriptomic expression atlases of PBMCs were subjected to transcriptomic validation with potential sample variability and experimental bias excluded (refer to Step III of this Example). The expression of aforementioned myeloid progenitor marker genes (CPA3, LYL1, GATA2, NFE2, and KLF1) showed elevated expression levels in peripheral blood myeloid subpopulations and significant up-regulation trends in mature myeloid cells. These results were consistent with progenitor-specific expression profiles identified via single-cell RNA sequencing (FIGS. 8A-8B, 10A-10B).

### Lymphoid and Myeloid Cell Lineage Expression Validation:

Critical transcription factors for hematopoietic stem cells (First Category in Step III of Example 3) exhibited ubiquitously high expression levels across four cell lines with statistically significant differences (e.g., RUNX1, CDK6, MYB, IMPDH2, CDK4; FIG. 12B, normalized to CDK6 expression in 293T cells [set as 1]), thereby validating their role as key determinants of fate specification in hematopoietic stem cells and lineage-committed blood cells.

Myeloid-associated transcription factors (NEF2, LYL1, GATA2, KLF1, GATA1) and the gene ALDH1A1 (Second Category in Step III of Example 3) demonstrated significantly elevated expression in myeloid cell lines K562 and MEG-01 (megakaryocytic lineage), with GATA1 showing a >3000-fold increase compared to non-myeloid controls (FIG. 12C; baseline expression defined in HEK293T).

Lymphoid lineage-associated genes (RUNX2, LST1, SPINK2; Third Category in Step III of Example 3) demonstrated significantly higher expression levels in the lymphoid cell line Jurkat compared to myeloid counterparts, exhibiting definitive lymphoid lineage-specific signatures (FIG. 12D; normalized to β-actin).

Monocyte-associated transcription factors (SPI1, MAFB, KLF4) and the gene LST1 (Second Category in Step III of Example 3) were markedly up-regulated in the monocytic cell line THP-1 (FIG. 12E; baseline expression measured in HEK293T).

NK progenitor-associated transcription factors (HOPX, DDIT4, ID2) showed prominent over-expression (q<0.01, FDR-corrected) in NK cell lines (FIG. 12F; RNA-seq TPM>50).

### Conclusion

Comprehensive validation of gene expression levels across PBMCs and hematopoietic cell lines confirmed that the expression profiles of transcription factors and marker genes (FIGS. 4, 6, 7) exhibited high concordance with transcriptomic expression profiles, exhibiting high lineage-specific expression signatures, thereby substantiating the accuracy and reliability of the proposed methodology of marker genes and transcription factors used for progenitor cell redefinition.

Validation of HPC Lineage-Specific Marker Genes in third-party single-cell transcriptomic expression atlases

PBMCs contain multiple cell subpopulations, while cell lines exhibit unique characteristics and can only partially reflect gene expression profiles. The use of quantitative PCR (qPCR) to validate complex cell populations has significant limitations, particularly in its inability to verify individual or rare progenitor lineage-specific genes. Therefore, representative or newly identified marker genes (Table 1) were validated and their expression landscapes were profiled within HSC transcriptomes.

Distinct lineage marker gene expression patterns were observed, such as:

DNTT, which is exclusively expressed in common lymphoid progenitors (CLP).

CSF1R, IRF8, and MAFB, which are specifically expressed in monocytic progenitors.

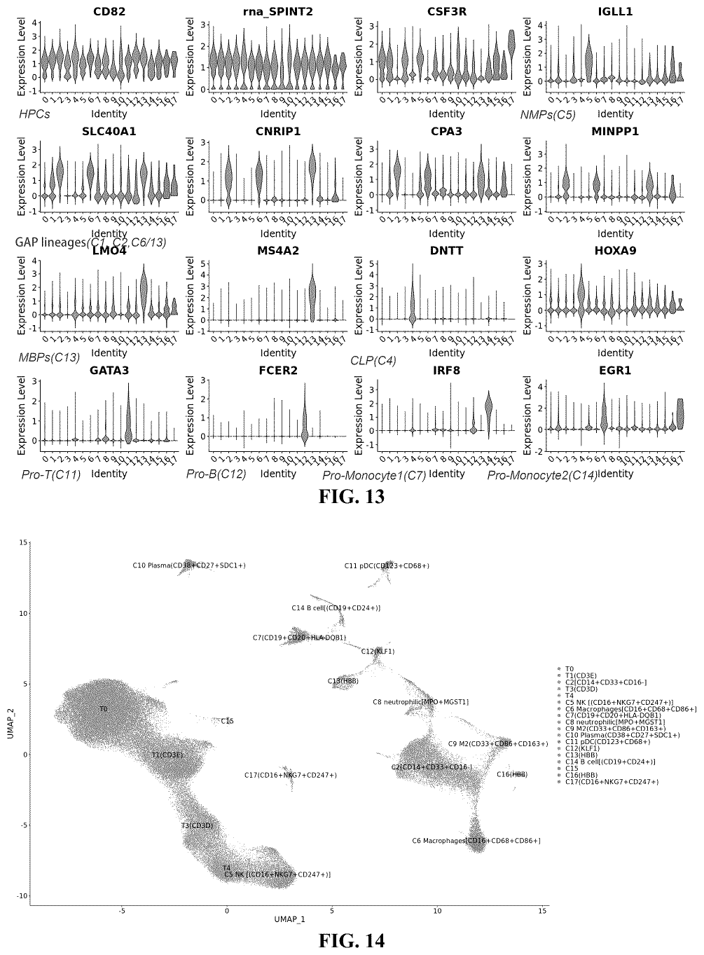

KLF1 and GATA1, which are uniquely expressed in mid-to-late-stage megakaryocyte-erythroid progenitors (FIG. 13).

The specificity of expression distribution and abundance indicates that the identified marker genes are reliable and representative (FIGS. 4 and 13). These marker genes for various HSC lineages and developmental stages exhibit clear stage-specific and lineage-specific characteristics.

Third-party public datasets with large-scale and high-cell-count data were selected as control data to exclude potential experimental and sample biases, thereby validating the accuracy and reliability of the lineage identification and definitions established in this invention. Single-cell data from multiple adult PBMC samples, representing terminally differentiated hematopoietic stem cells, were utilized in a comparative analysis to validate the identified lineage-specific marker genes.

Two single-cell PBMC datasets, GSE120221 and GSE96583 (including GSM2560245, GSM2560246, GSM2560247), were downloaded from NCBI's Gene Expression Omnibus (GEO) and integrated as a control group (data processing followed standard protocols, as referenced in Example 3). A total of 71,842 single-cell samples were obtained for the control group. Cell subpopulations were annotated and defined based on annotated subpopulation genes and well-known marker genes (FIG. 14) with known analysis protocol in the art.

### Validation of Identified Lineage-Specific Marker Gene Expression in Control PBMC Data

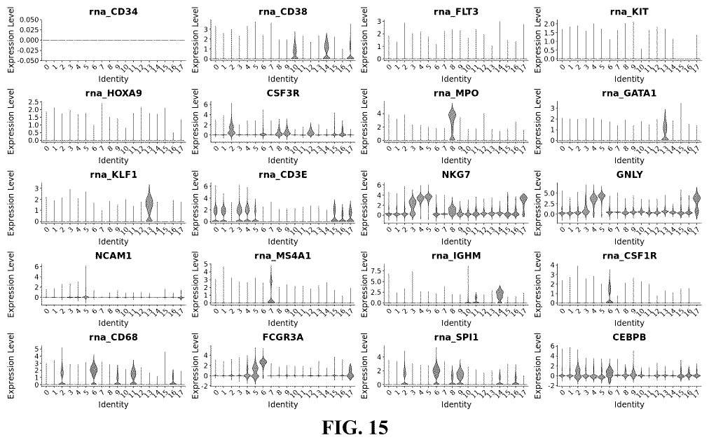

The control group PBMC data validating the expression profiles of identified genes demonstrated the following characteristics (FIG. 15):

CD34 was negatively expressed, representing mature hematopoietic cell populations.

HOXA9 was minimally expressed and identified as a lymphoid HSC-specific transcription factor.

MPO and CSF3R were co-expressed in mature neutrophil subpopulation C8 (FIG. 14).

GATA1 and KLF1 were expressed in megakaryocyte-erythroid subpopulation C13 (HBB positive).

NK cell markers NKG7 and GNLY were expressed in NCAM1-positive subpopulations C5 and C17.

Monocyte-macrophage populations positively expressed CSF1R, CD68, and FCGR3A (CD16), with transcription factors SPI1 and CEBPB showing lineage-specific expression (FIG. 15).

Comparative analysis with third-party data demonstrated that the marker genes of the aforementioned mature blood cell types were consistent with the expression profiles of the progenitor cell lineage-specific markers identified in this embodiment. Therefore, through comparative analysis of the single-cell data in this embodiment and control group data, the reliability and accuracy of the progenitor lineage identification and redefinition-redefined via transcription factors and lineage-specific marker genes-were validated.

### Reliability of Single-Cell Transcriptomic Expression Atlases for HSC

Single-cell sequencing technology has been widely applied to identify differentially expressed genes and novel distinct cell subpopulations. The reliability of these technical datasets has gained widespread recognition, particularly in the identification of critical cell subpopulations and their signature genes.

The preceding embodiments of the present invention confirmed the following:

Hematopoietic stem cells (HSCs) and progenitor cells undergo three major distinct lineage differentiation trajectories, each with distinct characteristics.

Stage-specific characteristics are observed within the same lineage differentiation trajectory.

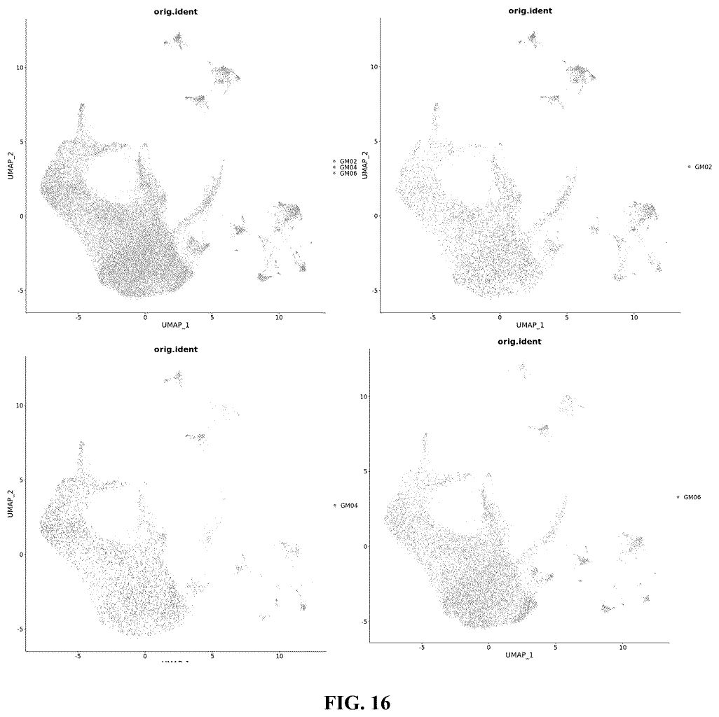

Comparative reproducibility analysis of single-cell sequencing samples revealed high consistency across multiple samples in terms of cellular distribution patterns, clustering quantity, and lineage trajectories, indicating excellent data reproducibility and consistency (FIG. 16). Both specific cellular subset distribution and clustering reproducibility were observed. The data obtained from single samples and multiple samples exhibit consistent differentiation trajectories, stages, and lineage distributions of HSCs and progenitor cells, validating the accuracy and reliability of the expression profiles.

The HSC expression profiles and signature characteristics has been historically hampered by extreme scarcity of HSCs, resulting in challenges in obtaining stable and reliable data. The statistical analysis of the cell counts across progenitor cell subsets in this embodiment revealed that the key cell subpopulations identified by the present invention—C0, C1, C2, C3, and C4—all exhibited cell counts exceeding 1,000. The cell counts for subpopulations C0-C16 were as follows: 6,505, 4,533, 3,726, 3,094, 1,781, 1,654, 1,147, 950, 772, 656, 617, 454, 449, 420, 403, 306, and 77, respectively. In this context, C15, C16, and C17 are erythrocytes or unclassified cell clusters (their exclusion via parameterized filtering does not affect the expression profile characteristics of other subpopulations). Based on the technical principles of single-cell sequencing, where individual cells are treated as discrete events, the expression profiles and defining features of key hematopoietic stem cells and progenitor cells identified in this embodiment may be regarded as demonstrating exceptional reproducibility and consistency with over 1,000 experimental replicates. Consequently, the findings exhibit high credibility and reliable data integrity.

### Summary of Examples 1-4

In summary, as demonstrated by the aforementioned Examples 1-4, the present invention has enriched and identified highly reliable, consistent, and reproducible expression and characteristics profiles of HSCs and multi-lineage progenitor cell subpopulations. Furthermore, it has identified lineage- and stage-specific transcription factors and genes governing fate determination in HSCs and progenitor subpopulations, while also providing a novel framework for the reclassification, precise definition, and signature gene characterization of progenitor cells.

In contrast to conventional classification and definition methods based on HSC surface markers (CD49f, CD38, FLT3/CD135, KIT/CD117), the present invention employed unsupervised clustering approaches that enable accurate and reliable classification, identification and redefinition of HSCs lineages and progenitor cell subpopulations based on highly enriched HSCs captured in Example 1. This approach eliminated subjective biases through a data-driven methodology (Examples 2 and 3). The reliability and accuracy of marker genes, transcription factors, and expression profiles were validated through multiple approaches (Example 4), including:

PCR verification of peripheral blood and cell lines (Example 4, Step I).

Comparative verification of expression profile data with third-party datasets (Example 4, Step II).

Evaluation of the data reliability of expression profile generated by this invention.

These validations comprehensively demonstrated the reliability and accuracy of the redefined progenitor cell expression profiles, confirming the precision and credibility of the progenitor cell lineage subpopulations and fate-determining genes identified and defined by the present invention.

Building on the advantages and validated outcomes of the aforementioned enrichment, identification, and definition methods, the present invention unambiguously identified transcription factors and fate-determining genes of key HSC lineages. A detailed list of genes is provided in Example 3 (Table 1). Notably, while some genes are known markers, the genes identified in this invention incorporate not only expression profiles but also differentiation stage-specific and direction-specific patterns in stem/progenitor cells. Crucially, although some genes are previously documented, their characteristics in progenitor cells—including novel lineage-specific signatures, differentiation stage-specific signatures, and unique expression dynamics—represent groundbreaking discoveries. For example, GATA2, a known myeloid transcription factor in HSCs, is for the first time precisely defined herein in terms of its emergence timing during differentiation, temporal expression trends, and spatial distribution patterns.

### Example 5: Validation of Human Lymphoid Hematopoietic Progenitor Cell Differentiation Potential

To assess the lymphoid differentiation potential of enriched HSCs, an in vivo transplantation experiment was conducted using immunodeficient mice.

### Experimental Methods

Experimental Model: Immunodeficient mice (6-8 weeks old) of the M-NSG strain (NOD-Prkdc^{scid}Il2rg^{em1}/Smoc) from Shanghai Model Organisms Center (China) were used. Mice were irradiated with a dose of 2-3 Gy prior to transplantation.

### Cell Transplantation:

a) The enriched HSCs from Example 1 were resuspended in PBS.
b) The cell suspension (50,000-100,000 cells per mouse) was administered via tail vein injection into irradiated mice.

### Post-Transplantation Monitoring:

a) Eight weeks post-injection, blood was collected from the submandibular vein for flow cytometry analysis.
b) Blood samples were collected every two weeks thereafter.

### Flow Cytometry Analysis:

a) CD19-positive lymphocytes differentiation status and proportion were analyzed to identify and validate lymphoid progenitor cells.

### II. Experimental Results

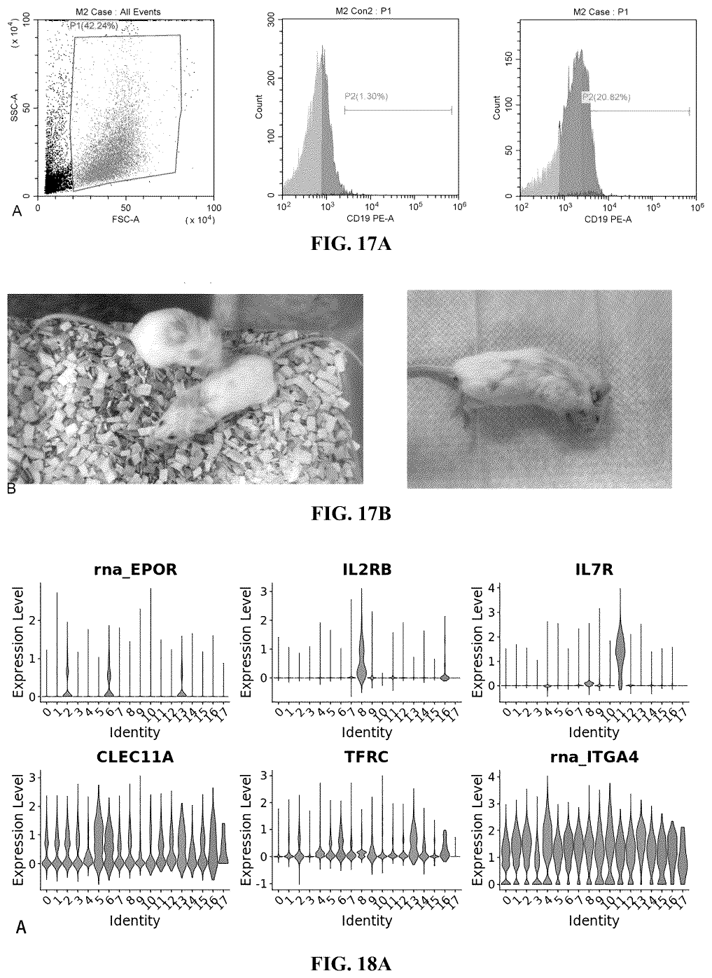

8-16 weeks post-transplantation, flow cytometry analysis of submandibular blood samples showed a significant increase in CD19-positive cells, indicating robust lymphoid differentiation potential of the enriched HSCs (FIG. 17A).

These findings confirm that the enriched HSC population possesses true hematopoietic stem cell characteristics and further validate the reliability of the progenitor cell enrichment method used for single-cell sequencing and gene identification.

Since the HSC enrichment method in Example 1 used a negative deletion approach, the obtained cell suspension was not composed exclusively of pure HSCs. Consequently, at later transplantation stages, some mice exhibited graft-versus-host disease (GvHD) phenotypes (FIG. 17B).

Based on the preceding Examples 1-4, the present invention successfully and accurately identified various types of hematopoietic progenitor cells, clarified differentiation pathways, lineage directions, and key nodes. Additionally, genes governing progenitor cell fate determination were identified. This innovative approach overcomes long-standing challenges in hematopoietic progenitor identification and fate determination. In summary, by unraveling the complexity of HSC composition, defining their characteristics, differentiation determinants, and pathways, precise control and application of HSCs become feasible.

Furthermore, this invention integrates hematopoietic progenitor characterization, fate-determining gene expression profiles, and the hierarchy of hematopoiesis (see Example 9), enabling diverse applications. The following sections will elaborate on potential applications and their significance.

### Example 6: Application of Human HPC Marker Genes in Induced Differentiation and Lineage Differentiation Tracking and Localization

HSCs are scarce, and obtaining large quantities from umbilical cord blood or bone marrow is challenging. While in vitro culture has made progress over the years, its large-scale clinical applications remain significantly challenged due to limitations in growth cycle and amplification potential. Therefore, understanding the fate determination and influencing factors of HSC differentiation is critical for advancing in vitro HSC culture, especially for isolating specific progenitor cell types and achieving precise culture, induction, and differentiation control at different lineage stages.

### I. Validation of Progenitor Cell Differentiation and Induction

In Example 3 (Step IV), marker genes and transcription factors defining newly characterized hematopoietic progenitor cell lineages were identified (Table 1). This embodiment validated and elucidated the application of expression profiles of growth factor receptors and marker genes in lineage-specific cells for the induction, differentiation, and culture of progenitor cells.

In previously reported implementations, the neutrophil receptor CSF3R—a receptor for the stimulatory factor G-CSF—has been extensively utilized to promote neutrophil proliferation and peripheral blood mobilization. Expression profiling results demonstrated its specific expression in early MPC progenitors and neutrophil-monocyte progenitors (FIG. 13), consistent with its established application characteristics. CSF1R, the receptor for M-CSF (an essential growth factor in monocyte culture), is specifically expressed in macrophage progenitors (Pro-monocytel, C7) within the monocytic lineage, as evidenced by expression profiling in FIG. 4.

Growth factor receptor expression profiling reveals lineage-specific patterns:

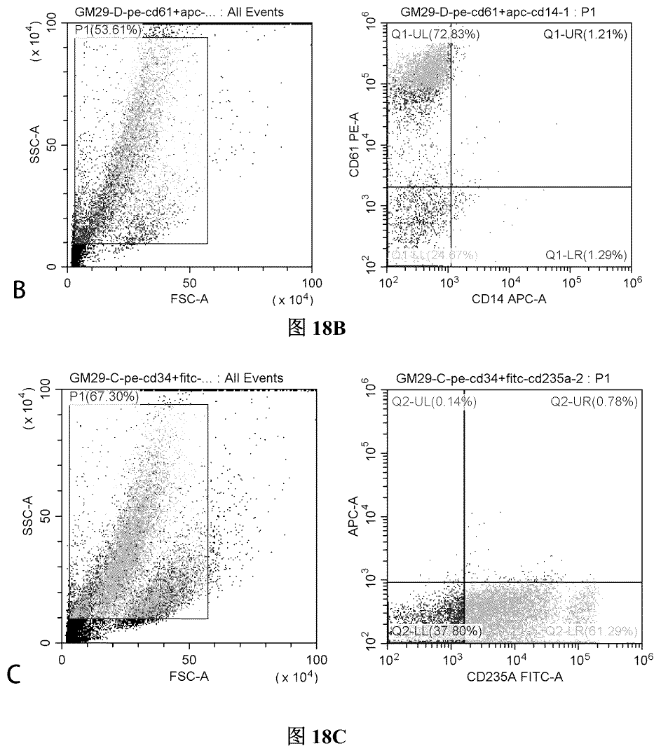

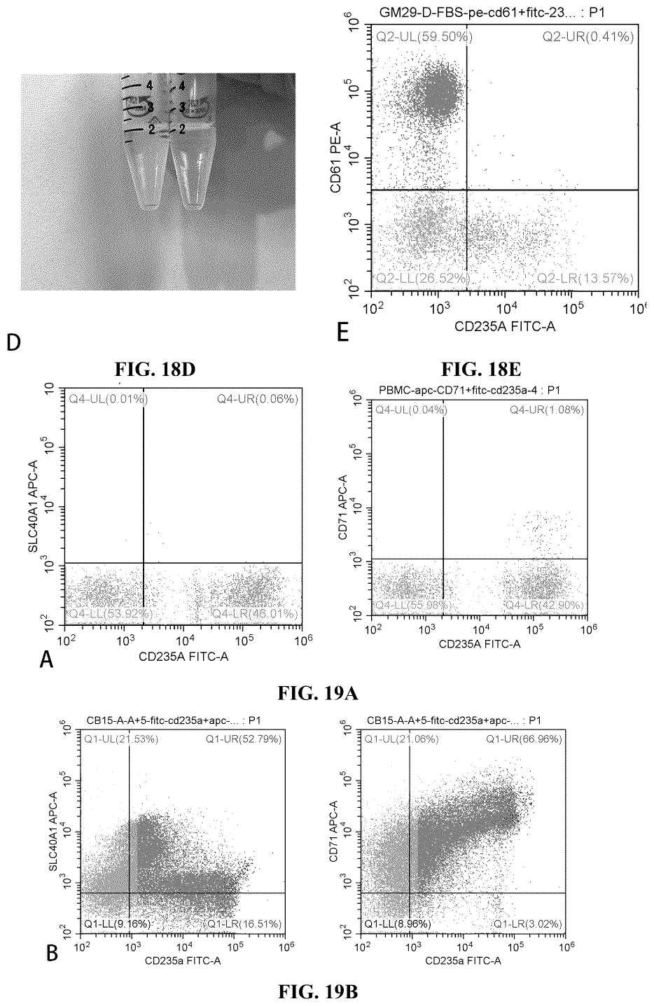

The IL7 receptor (IL7R), critical growth factor for lymphocyte development, shows highly specific expression in T-lymphoid progenitors (FIG. 18A);

IL2RB, the receptor for IL15 (a key growth factor for NK cells), is specifically expressed in NK progenitors (FIG. 18A);

EPOR, the receptor for erythropoietin (EPO, a key growth factor for erythrocytes), exhibits exclusive expression in megakaryocyte-erythroid progenitors (FIG. 18A);
SCGF (CLECI1A), a burst-promoting factor for erythroid colonies, is highly expressed in erythroid progenitors (FIG. 18A).

The in vitro induction and differentiation of hematopoietic progenitor cells has been extensively studied, with numerous well-established culture systems. The application of growth factors required for induction culture of the aforementioned lineage-specific cell populations is well-characterized, consensus-recognized, and extensively validated through years. Our newly identified expression profiles and established redefinition for progenitor cells in this invention (Table 1) aligned with the well-characterized applications of growth factors in the field. Additionally, the expression profiles of growth factor receptors exhibit precise concordance with lineage-specific definitions of progenitor cells—as exemplified by the exclusive expression of IL2RB in the newly defined NK progenitor subpopulation. These results further corroborate the accuracy and potential applications of the redefined progenitor subpopulations/expression profiles in cell induction and differentiation. Based on the reliable expression profiles of known receptors and genes, we can effectively identify the growth factors and supplementary substances required for novel progenitor lineage induction and differentiation. In specific embodiments, for instance, the newly identified SLC40A1 is associated with iron metabolism, and its high expression characteristic aligns with the substantial iron demand for erythrocyte growth. Additionally, ITGA4, which is widely expressed across all types of progenitor cells (FIG. 18A), serves as a fibronectin receptor. Therefore, incorporating fibronectin into hematopoietic progenitor cell cultures promotes the growth and developmental processes of hematopoietic cells.

Additionally, the stage-specific and direction-specific characteristics of the expression profiles established in this invention provided practical guidance for the timing and stages of growth factor utilization. Specific implementation examples include:

The late emergence of IL2RB expression suggested that supplementation should be initiated only upon differentiation into the NK precursor progenitor stage.

FLT3 exhibits minimal or negative expression in megakaryocyte-erythroid progenitors (FIG. 4); therefore, supplementation with FLT3L ligand is not required in their induction culture.

The KIT gene, encoding the receptor for stem cell factor (SCF), showed elevated expression during late branching stages of megakaryocyte-erythroid differentiation, particularly in mast cell progenitors (FIG. 4). Consequently, SCF dosage should be reduced in the later phases of megakaryocyte-erythroid induction culture to effectively suppress mast cell differentiation.

By leveraging the expression profiles of growth factor receptors, ligands, or metabolic intermediate genes across progenitor subpopulations, the addition of corresponding growth factors, chemokines, ligands, or intermediates during in vitro culture can refine methodologies and systems for progenitor cell expansion. This approach provides stage-specific and dosage-guided optimization, enabling precise induction and enhanced differentiation control in vitro.

This invention can be used for in vitro induction culture of hematopoietic progenitor cells and system optimization. Specifically, by clarifying of the gene expression profiles and characteristics of the various progenitor marker genes, growth factor receptors and metabolism-related ligands, in combination with hematopoietic hierarchy structure, this invention enable optimize or select for in vitro culture components or systems, ultimately achieving improvement in in vitro induction. In summary, the expression profiles and the redefined progenitor marker genes (e.g., growth factor receptors and ligands) in this invention enable precise guidance for lineage-specific progenitor induction, differentiation, and developmental regulation. Current applications of hematopoietic stem cells (HSCs) focus on anti-aging and immune enhancement. The progenitor cell atlas and expression profiles established herein will advance large-scale in vitro expansion of HSCs, accelerating their utilization in health management strategies such as anti-aging therapies and personalized immunity enhancement.

II. In Vitro Induction of Erythrocyte Differentiation from HPCs: Verification of Differentiation Direction and the Application of SLC40A1 in Lineage Differentiation Stage Tracking and Localization

During the in vitro induction of erythrocyte differentiation, some cells differentiate into mast cells, sharing key transcription factors such as GATA1 and KLF1. Additionally, megakaryocytes and erythrocytes originate from a common progenitor cell type. These two points are well-documented in hematopoietic differentiation studies. The previous examples demonstrate that megakaryocyte-erythroid progenitors (MEP) serve as common progenitors for both megakaryocytes and erythrocytes. During the third stage of differentiation, they generate branch cells known as MBPs (mast cell-basophil progenitors), whose expression and differentiation characteristics align with known hematopoietic differentiation features, confirming the accuracy of the defined differentiation stages and pathways of megakaryocyte-erythroid progenitors described in this invention.

To further validate the lineage differentiation tracking and localization application of SLC40A1, a gene from the marker gene list (Table 1), an example verification was conducted for its role in the differentiation stages of erythroid progenitor cells. CD71 (TFRC) is a well-established surface protein commonly used for detecting erythrocyte differentiation. Its expression disappears as erythrocytes mature, making it a widely used marker for erythroid differentiation stage localization. However, due to its lack of specificity (FIG. 18A), as it is also expressed in common lymphocytes, its effectiveness in distinguishing erythrocyte differentiation stages is limited.

### Experimental Procedure

### Hematopoietic Stem Cell Enrichment:

1. Hematopoietic stem cells were enriched from 1.5 mL of umbilical cord blood using the protocol described in Example 1 (STEMCELL, 15026), resuspended in PBS.
2. In Vitro Culture of Hematopoietic Stem Cells:
3. The cells were cultured in hematopoietic stem cell medium (StemSpan™ SFEM II, Catalog #09605).

### Induction Culture Conditions:

Three Different Culture Conditions were Tested:

Culture System A: Megakaryocyte culture medium (StemSpan™ M, Catalog #02696) containing rhSCF, TPO, and rhIL-6.

Culture System B: Erythroid induction culture medium (StemSpan™ SFEM II, Catalog #09605) containing rhSCF (10 ng/mL, HY-P70781, MCE), IL3 (10 ng/mL, Nearshore Protein), EPO (20 ng/mL, C001, MCE), as well as additional components TSI (PB180429, Procell) and fibronectin (1 μg/mL, FN F8180, Solarbio).

Culture System C: Combined megakaryocyte and erythrocyte induction culture medium (StemSpan™ M, Catalog #02696) supplemented with EPO.
4. Flow Cytometry Analysis:
After 7 to 25 days of induction culture, the following parameters were analyzed:

### Cell Morphology and Growth Status

Expression and changes in various progenitor cell markers:APC-CD34 (hematopoietic stem cells) (343608, BioLegend); PE-CD61 (megakaryocyte progenitor cells) (336406, BioLegend); FITC-CD235A (erythrocyte marker) (306610, BioLegend); APC-CD71 (551374, BD); Alexa Fluor 647-SLC40A1 (FAB92924R, R&D). The changes of aforementioned parameters were used to identify and verify the differentiation ability of megakaryocyte-erythroid progenitor cells, and the application of the marker gene SLC40A1 in tracking and localizing the stage of lineage differentiation.
5. Peripheral Blood Analysis:
Mature erythrocytes were isolated from peripheral blood, and the expression of SLC40A1 was analyzed by flow cytometry.

### Experimental Results

The validation results demonstrated that megakaryocyte-specific medium (StemSpan™ M, Catalog #02696) effectively induced the generation of a high proportion (72.8%) of CD61+ megakaryocytes (FIG. 18B), indicating that hematopoietic stem cells (HSCs) preconditioned with SCF (KIT receptor ligand) exhibit differentiation potential toward the megakaryocyte-erythroid lineage (FIG. 18B). Upon stimulation with erythroid lineage-specific growth factors (EPO and SCF, targeting receptors EPOR and KIT) for 7 days, the cultured progenitor cells exhibited robust proliferation. Flow cytometry analysis revealed a significant decrease in the proportion of CD34+ progenitor cells by day 7, confirming substantial differentiation. By day 14, the proportion of CD235A+ erythroid cells increased dramatically to 61.3% (FIG. 18C), and the primary cell pellet displayed a characteristic red hue (FIG. 18D), consistent with hemoglobinization. Notably, dual induction using a combined megakaryocyte-erythrocyte culture medium simultaneously generated CD235A+ erythroid cells (13.6%) and CD61+ megakaryocytes (59.5%) (FIG. 18E). This observation establishes that both lineages can originate from a common progenitor population, corroborating the lineage differentiation trajectory proposed in Example 3.

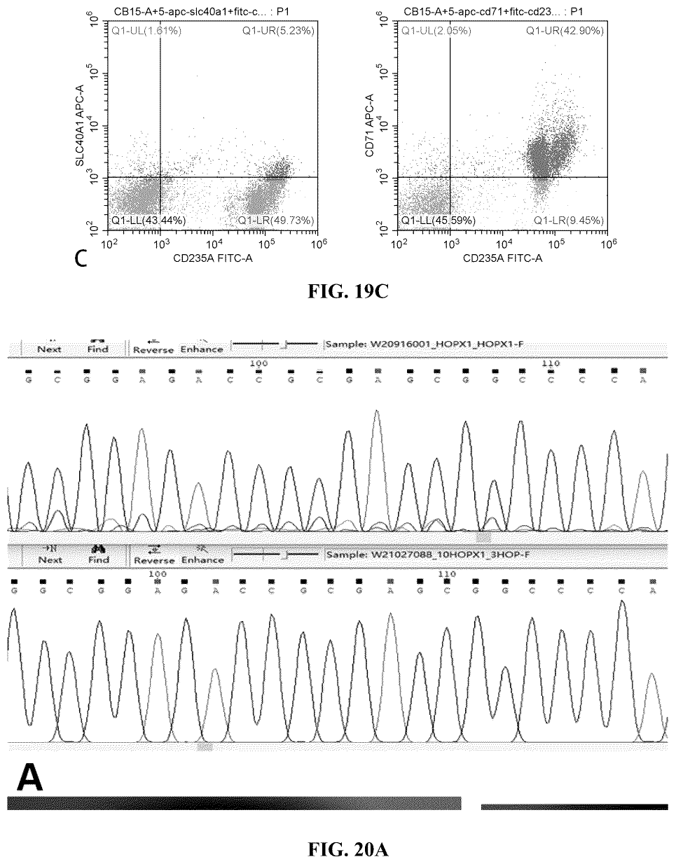

Flow cytometry analysis of whole peripheral blood revealed that within the CD235A+(erythroid marker) cell population, SLC40A1 expression was undetectable, and negligible expression (0.06%) was observed in PBMCs. Additionally, CD71 was also absent in these populations (FIG. 19A). These findings indicate that SLC40A1 serves as a stage-specific marker gene during progenitor cell differentiation, with its expression pattern aligning precisely with transcriptomic profiles and demonstrating superior specificity compared to CD71.

Flow cytometry analysis of SLC40A1 expression dynamics at Day 3, 7, and 14 during erythroid induction revealed distinct differentiation stages:

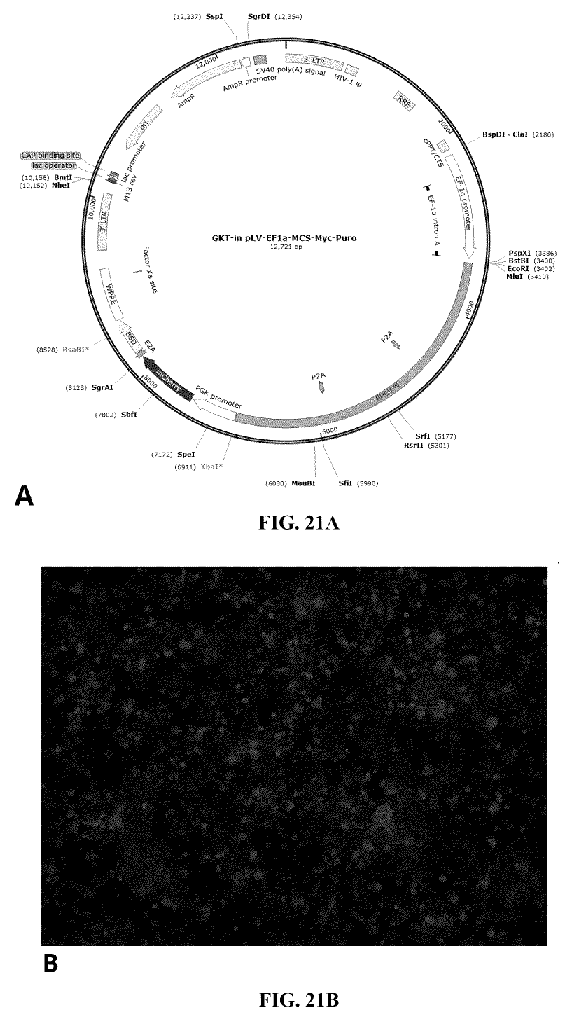

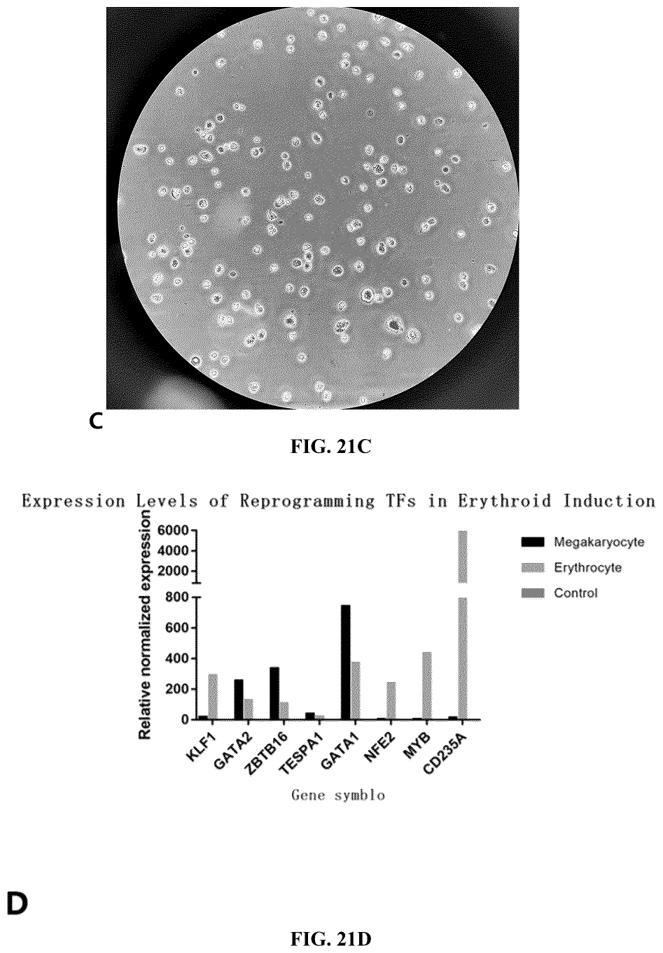

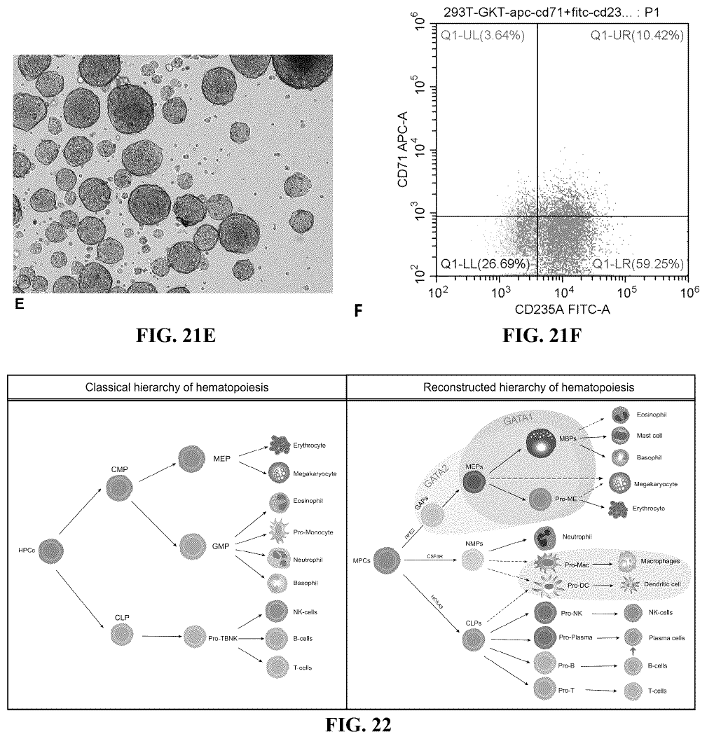

Day 3: SLC40A1 exhibited a tri-modal distribution in flow cytometry profiles (FIG. 19B), indicating heterogeneity in erythroid subpopulations at early differentiation stages. Morphologically distinct erythroblast subsets at these stages are shown in FIG. 21C.

CD71, a classical marker, remained persistently positive throughout differentiation, with only quantitative changes in expression levels, demonstrating its limited utility in stage discrimination compared to SLC40A1. This suggests that most cells at this phase were early progenitors (proerythroblasts/basophilic erythroblasts).

Day 14: SLC40A1 expression declined to undetectable levels, while mature erythroid subsets displayed a CD235A+SLC40A1-CD71+ profile (FIG. 19C). The disappearance of SLC40A1 expression, concomitant with down-regulation of CD71 and up-regulation of CD235A, indicates that the cells have progressed into the polychromatic erythroblasts or orthochromatic erythroblast stages of differentiation (FIG. 19C).

Terminal Maturation: Complete loss of SLC40A1 and CD71, accompanied by enucleation, marked the transition to reticulocytes and mature erythrocytes (FIG. 19A).

### Specific Application in Lineage Tracking and Localization

Flow cytometric profiling of SLC40A1 dynamics during in vitro erythroid induction revealed stage-specific expression patterns:

Early hematopoietic progenitors (CD34⁺): SLC40A1 expression is low/undetectable.

Induction phase: SLC40A1 expression increases, marking transition to early erythroid differentiation stages (proerythroblast or basophilic erythroblast stages; CD235A+CD71+SLC40A1+).

Terminal maturation phase: CD235A-high populations expand, while SLC40A1 becomes undetectable (CD235A+CD71+SLC40A1−), indicating entry into late differentiation (polychromatic/orthochromatic erythroblast stages).

This embodiment of erythroid induction corroborated findings from Example 3, demonstrating that lineage differentiation direction- and stage-specific biomarkers (Table 1), when combined with the expression profiles and dynamic changes of one or more markers (e.g., CD235A, CD71, SLC40A1), enable precise discrimination of HPC differentiation stages, thus can be applied to track and localize HPCs during differentiation. Therefore, by detecting the expression profiles (via antibody-based assays) and dynamic changes of characteristic genes, the differentiation stages of progenitor cells can be precisely mapped. Furthermore, lineage-specific genes are labeled using methodologies such as gene editing, fluorescent tagging, or molecular labeling. Subsequent detection and tracing of the expression profiles and dynamic changes of these labeled molecules during progenitor differentiation can define the stages and directions of HPCs differentiation, thereby enabling tracking and spatial-temporal localization of progenitor cell lineages. Therefore, once cell types are defined (identified and classified), their marker genes are validated (cellular signatures), and their differentiation pathway and hierarchy (spatial-temporal positioning and stage specificity) is resolved, a method for lineage-specific localization and tracking of progenitor cells can be established. This method integrates features of HPCs, expression profiles of marker genes, and hematopoietic hierarchy (see Example 9) to detect or trace the dynamic expression patterns of lineage-specific genes. Specifically, this involves labeling lineage-specific genes via gene editing, fluorescent tagging, molecular labeling, or analogous techniques. By monitoring and tracking the temporal-spatial dynamics and expression changes of these labeled molecules during progenitor differentiation, the method achieves precise tracking and localization of progenitor cell populations.

### Example 7: Application of Transcription Factor and Fate-Determining Gene Intervention in Human HPC Subpopulations for Immunotherapy and Target Selection

This example illustrated and validated the application value of transcription factors, genes, and expression profiles in immunotherapy regimen such as immunotherapy, CAR-T therapy target development, PD-1-based treatments, and precision tumor therapy.

Abnormal differentiation and proliferation of HSCs are key factors leading to leukemia. The aforementioned examples have identified lineage-specific key transcription factors governing hematopoietic stem cell (HSCs) fate determination. Therefore, intervening in these key transcription factors and fate-determining genes can regulate the growth and differentiation of specific hematopoietic lineages, allowing precise control over differentiation and proliferation of HSCs. This provided an effective approach for developing leukemia treatments. Consequently, intervening lineage-specific key transcription factors and fate-determining genes holds significant potential for treating related hematologic malignancies. In this embodiment, we performed transcription factor knockout experiments in the NK92 and K562 tumor cell lines, as well as the Jurkat lymphoid tumor cell line, to verify the therapeutic potential of key transcription factors of identified progenitor cells in cancer treatment.

Methods of intervening transcription factors and fate-determining genes as mentioned above include(s) any one or more of gene overexpression, endogenous gene activation, gene expression inhibition, gene editing, gene knockout, and exogenous activation, wherein endogenous gene activation includes one or more of activation or inhibition via small molecules or drugs, co-culture activation, growth factor and inflammatory factor activation, gene editing, substitution of gene function-related intermediate metabolites, delivery of ligands, antibodies, and vector. Furthermore, intervening transcription factor can be achieved by constructing lentiviral or adenoviral systems of overexpression, small RNA (siRNA) interference-based inhibition of transcription factor, or gRNA lentiviral or adenoviral systems of CriSPR-Cas9-based knockout systems of expression of transcription factor.

I. Construction of gRNA Gene Knockout Vectors

The gRNA target sequence in the pCas001-LentiCRISPR V2-U6-sgRNA-HOPX-Cas9-p2A-puro vector (documented in Neville E. Sanjana, Ophir Shalem, Feng Zhang, “Improved vectors and genome-wide libraries for CRISPR screening,” Nat Methods, 2014 August; 11(8):783-784) was replaced with GACCGCGAGCGGCCCCACAG (SEQ ID No. 1), creating a gRNA knockout vector for the transcription factor HOPX.

Similarly, we constructed gRNA knockout vectors for other key transcription factors in HSCs, such as SPI1, ETV6, BCL11A, RUNX1, CDK4, in myeloid hematopoietic progenitor such as NFE2, LYL1, MYB, TESPA1, GATA2, KIT, KLF1, TAL1, and ZBTB16, as well as in lymphoid hematopoietic progenitors such as JUN/FOS, RUNX2, HOXA9, TCF4, DDIT4, BASP1, HOPX, KLF10, HOXA3, and TSC22D1. The gRNA design was performed using the CHOPCHOP tool (http://chopchop.cbu.uib.no/). The full list of transcription factors and genes is detailed in Example 3, Step III, and Table 1.

### II. 293T Cell Culture and Lentivirus Packaging

1. 293T cells were cultured in DMEM with 10% FBS until reaching 60-70% confluence.

2. One hour before transfection, the medium was replaced with 10 mL of serum-free medium.

3. Preparation of mixtures of target plasmid, packaging plasmids and transfection reagent:
a) 1200 μL Opti-DMEM (serum-free) was mixed with 10 μg target plasmid (HOPX<500 ng/μL) and 12 μg viral packaging plasmids (psPAX2 and pMD2.G) at a weight ratio of 5:4:2 (10 μg:8 μg:4 μg) in sterile 5 ml EP tubes, fully mixed.
b) 66 μL PEI (1 μg/μL) transfection reagent was added at a volume ratio of the plasmid and PEI of 3:1, and incubated at room temperature for 15 min.
c) Culture dish was fetched out, and the prepared transfection mixture was added in culture dish, gently mixed, labeled, and incubated in incubator.

4. After 6-8 hours, the medium was removed, washed by 4 ml PBS once, then replaced with fresh complete medium, for 48 hours

5. Viral supernatants were collected at 48h and 72h after transfection (collected at 24h, replaced by fresh medium), centrifuged at 2000 g for 30 min at 4° C. after collecting, supernatant was collected, filtered with a 0.22 μm filter, and stored in 40 ml ultracentrifuge tubes.

6. Virus concentration: the viral supernatant was mixed with PEG8000mix at a ratio of the viral supernatant: PEG8000mix=10:1, add the PEG8000mix mixed liquor: [PEG8000(10×): 20× NaCl (3 mol/L)]=1:20, or the Lenti-X Concentrator was used to concentrate and added at a ratio of Lenti-X Concentrator: viral supernatant=1:3; shaking up and down at 4° C. overnight and concentrated.

7. Centrifugating at 2000 g for 20 min at 4° C., supernatant was removed, and resuspend with 100 μL PBS, put on ice for 30 min. And it was put in 1.5 ml tubes after resuspension, storing at −80° C.

### III. Infection of Target Cells

1. Target cells in condition (NK92 tumor cells) were seeded at 1×10⁵ cells/mL in 6-well plates, the numbers of inoculated cells were slightly different due to the growth rate of the cells, generally ensuring 50-70% confluence at the time of viral infection the next day.

2. The most commonly used working concentration of polybrene is 6˜8 μg/ml (diluted with Opti-DMEM), generally 6 μg/ml.

3. Plated the cells according to the experimental requirements (such as 12-well plate). The density of cells was about 50% on the second day. The cells were incubated overnight at 37° C.

4. Prepared the mixture of complete medium and Polybrene (Solebo), and the final concentration of Polybrene was the optimal final concentration (6 ug/ml) after exploration. Before infection, removed the virus from the refrigerator and quickly melt it in a 37° C. water bath, Polybrene was diluted in Opti-DMEM, and the desired virus stock was added, mixed, and stayed at room temperature for 15 min. The original cells medium was sucked out, and ½ volume of fresh medium was added, and 1 ml Polybrene/medium mixture was placed in each well.

5. On the day after infection (about 24 hours), the culture medium containing virus was sucked out and replaced with fresh complete culture medium, and the culture was continued at 37° C. Between 48 and 72 hours after infection, the efficiency of GFP expression could be observed by fluorescence microscopy for viruses with GFP reporter gene, and for viruses with Puromycin resistance gene, fresh complete culture medium containing appropriate concentration of Puromycin was replaced, stably transduced cell lines were selected, finally stable HOPX knockout NK92 cell lines were obtained.

6. Genomic DNA was extracted, and Sanger sequencing was conducted to verify gene knockout efficiency.

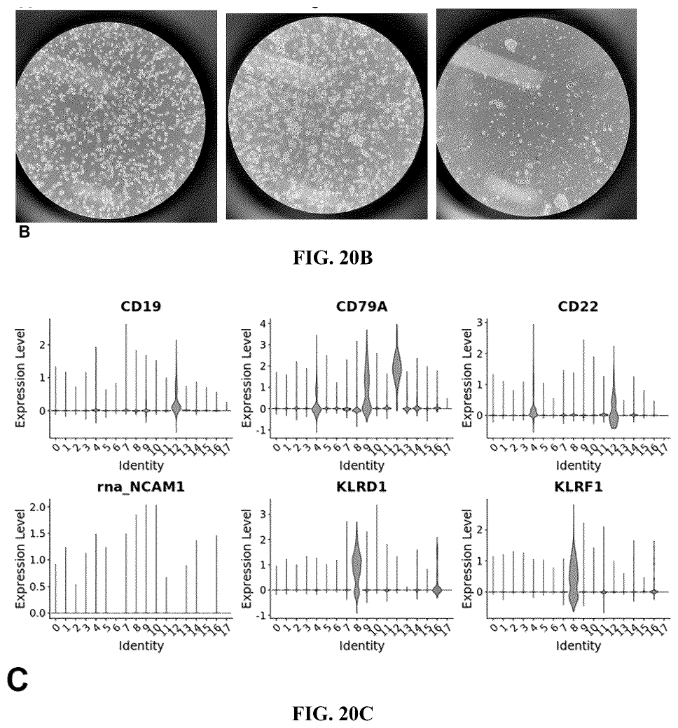

Sequencing results showed that: After transient transfection of cells with the lentiviral vector containing the gRNA sequence of the HOPX gene GACCGCGAGCGGCCCCACAG (SEQ ID No.1), mixed peaks were observed at the target site in the sequencing chromatogram (FIG. 20A), demonstrating effective HOPX gene knockout by the gRNA.

### IV. Effect of HOPX Knockout on the Growth of NK92 Cancer Cell Line

Following the knockout of the key transcription factor HOPX in NK92 cells via effective vector of gRNA, after selection with puromycin, part of positive HOPX-knockout NK92 cells exhibited growth arrest in vitro, with a subset undergoing progressive apoptosis (FIG. 20B). This indicated that HOPX knockout effectively inhibits the growth of NK tumor cells and regulates the differentiation and expansion of NK progenitor cells, highlighting its therapeutic potential for targeting NK-cell malignancies.

The expression profiles of transcription factors and target gene described in Examples 3, 4, and 5 above have been verified to be lineage specific and to be critical fate determining factors during progenitor cell differentiation. Therefore, similarly, by intervening the transcription factors of corresponding lineage (listed in Table 1 and Example 3) in detailed embodiments, the growth inhibition, apoptosis, and the fate control of differentiation and proliferation of various HPCs are achieved. This has significant therapeutic potential for treating hematologic malignancies and related disorders.

One of the primary characteristics of hematologic malignancies and various hematopoietic cell abnormalities is the abnormality of HSCs, whose abnormal biological behaviors exhibit malignant clonal expansion, proliferation, metastasis, and invasion. Due to the heterogeneity of HPCs, their exact identification classification can't be carried out, and their expression profiles and pathways remain unclear, making that the genetic, molecular, and signaling abnormalities of abnormal biological behaviors of HPCs in cancer patients and various hematologic disorders remain inaccurate and vague. Since all blood cells originate from progenitor cells, the present invention has identified the genes of key transcription factors that determine the fate of specific hematopoietic lineages and branching sub-lineage progenitors (FIG. 8, FIG. 9). By regulating these genes, cell fate and biological behavior can be controlled, finally providing an application for disease treatment and management. Furthermore, based on the redefined HPC subpopulations, aforementioned gene expression profiles, and the established hematopoietic hierarchy (see Example 9), by comparing the characteristics of HPCs in hematologic disorders and that identified in the present invention, the classification of hematologic disorders can be clarified, and the key molecular mechanisms, signaling networks, gene or protein resulting in various abnormal biological behaviors of hematopoietic cells in patients are identified, for example, by comparing the expression profiles and cellular characteristics of megakaryocytic-erythroid progenitor cells (MEP cells) in this invention with those of MEP-lineage hematopoietic tumor progenitors, it can be identified that which genes or signals in which progenitor subpopulation(s) in the specific differentiation stages exhibit aberrant expression or activation that ultimately lead to pathological biological behaviors; This approach can further be applied to modulation, blockade, or disruption of the responding abnormal biological behavior that may attenuate, eliminate, or restore normal cellular function by targeting responding genes or signals, ultimately achieving disease control and treatment. In summary, through the groundbreaking innovation of this invention, the inventors have established a novel normal control cohort encompassing HPCs across lineages (including gene expression profiles, cellular characteristics, and hematopoietic hierarchy). By contrasting with these characters of progenitor cells in disease cohorts (hematologic malignancy patients), this methodology enables precise identification and clarification of key genes and pathogenic features driving cellular abnormalities.

Therefore, the substance described in the aforementioned embodiments is introduced in vitro into hematologic tumor cells to modulate molecules, signaling networks, and genes or proteins associated with abnormal biological behaviors, thereby reversing or altering the malignancy of the hematologic tumor cells. Alternatively, a prophylactically or therapeutically effective amount of aforementioned substance is administered to a subject to regulate said molecules, signaling networks, and genes or proteins associated with abnormal biological behaviors, thereby reducing or eliminating hematologic tumor cells in the hematopoietic of subjects, or diminishing their malignancy or reducing proliferation, metastasis, invasiveness, or inducing cell death.

Alternatively, HPCs obtained from the subject, and targeting abnormal characteristics of tumor progenitor cells, they are modified in vitro by tailoring aforementioned substance to reprogram the HPCs toward a “normalized” state. The reprogrammed cells are cultured to make the hematopoietic progenitor cells to differentiate into hematopoietic cells in a more normalized state, which are then reinfused into the subject. This rectifies defects in hematopoietic progenitor cells used for autologous hematopoietic stem cell transplantation, thereby improving therapeutic efficacy.

Ultimately, the invention achieves prevention or treatment of hematologic tumors and various hematologic disorders in subjects.

Therefore, based on the lineage-specific fate-determining genes, their expression signatures (expression profiles), and hematopoietic hierarchy described in the foregoing examples (3-4) (see Example 9), the invention provides a method to intervene the specific fate-determining genes of progenitor cell subpopulations through distinct substances, regulate growth inhibition, elimination, and control of differentiation and proliferation of corresponding progenitor cell subsets and mature cell populations, modulating malignancy or pathological biological behaviors in both progenitor and mature cells. The methodology holds therapeutic potential for treating and controlling various hematologic tumors.

Second, in the field of immunotherapy, the present invention develops more effective target genes and treatment regimens to new multiple hematologic tumor cell lineages. Specific embodiments include: B-cell lineage marker genes, such as CD19, CD79A, CD22 and so on, exhibit stage-specific expression patterns, CD19 is expressed only in late-stage progenitors and mature B lymphocytes (FIG. 20C); CD19-targeted CAR-T therapy effectively eliminates CD19+ mature B cells in diffuse large B-cell lymphoma (DLBCL), While it is unable to eliminate “seed” populations (progenitor cells) and CD19-negative progenitor cells during differentiation of B lymphocytes for the possible reason that CD19 is not expressed in early common lymphoid progenitors (CLPs, C4 subpopulation) and is lowly expressed in pro-B cells (C9), leading cancer cells to continue to expand and recurrence after treatment.

CD79A/CD79B genes, as demonstrated by the lineage, expression signatures of characteristic genes and hematopoietic hierarchy data of progenitor subpopulations identified in this invention, show high positivity in different steps throughout B-cell development, including plasmablast progenitors (FIG. 20C). Targeting CD79A thus provides superior efficacy and reduced recurrence risk in Immunotherapy for B-lymphoid tumors compared to CD19. Similarly, CAR-NK therapies relying on CD56 (NCAM1), which exhibits weak progenitor expression, show limited efficacy, alternative targets such as KLRF1 and KLRD1 (CD94) demonstrate improved therapeutic outcomes. In summary, the lineage-specific expression profile genes in progenitor cells identified in this invention (Table 1) enable more precise and effective selection of immunotherapeutic targets and regimens for various tumors (granulocytic, lymphoid, monocytic, erythroid, and other lineages). iii. Third, regarding PD-1 immune checkpoint-based therapies, the expression profiling data in this invention—featuring precisely redefined progenitor cell types and their differentiation stages—enables development of novel immune checkpoint molecules, and clarification of the expression profiling of immune checkpoint molecules, guiding development of enhanced immune function and improvement of immunomodulatory strategies.

Lastly, the expression profiling of signature gene in the present invention provides guidance and applications for the precise development of tumor therapeutic targets. In specific embodiments, such as FLT3, which is commonly used as a therapeutic target in myeloid leukemia (AML), the aforementioned experimental data demonstrate that FLT3 shows nearly negative expression across the entire spectrum of megakaryocytic-erythroid progenitor cells (FIG. 4), therefore, when considering therapies for megakaryocytic-erythroid lineage tumors or Hypertrophic-basophilic tumors in myeloid leukemia, FLT3 should be excluded as a therapeutic target, which guides precise medication and treatment. Similarly, CD38 is widely expressed across various progenitor cell populations, exhibiting lineage-specific differences in expression abundance, and its lineage-specific expression characteristics should be considered when selecting it as a therapeutic target.

Therefore, based on the lineage, expression characteristics of characteristic genes and hematopoietic hierarchy (Example 9) of progenitor subpopulations described in the aforementioned Examples 3-4), the present invention achieves a method for precisely selecting and optimizing characteristic gene targets of progenitor subpopulations. This approach is applicable to the optimization of therapeutic strategies, such as CAR (chimeric antigen receptor) design or small-molecule drug development. The therapeutic strategies include, but not limited to, various methodologies such as introducing regulatory substances into progenitor and mature cell populations, co-culturing with these cell populations, or modifying them with the aforementioned substances, all of which collectively facilitate more efficient and comprehensive growth inhibition, targeted elimination, or immune function enhancement for specific progenitor and mature cell subpopulations.

In summary, the characteristics of expression profile of each progenitor cell lineage in this invention, including characteristics, stages, lineage direction, etc., combined with the expression characteristics and hematopoietic hierarchy of mature cells of the same type, provide critical guidance and applied value for precision immunotherapy as well as therapeutic target and treatment regimen in hematologic tumors. Based on the characteristics of each progenitor cell lineage, this invention provides a method that by utilizing lineage-specific gene expression profiles of progenitor cells of the same type and their corresponding mature cells (e.g., the lineage-specific target gene CD79A of B progenitor cells and B cells), selecting corresponding characteristic target genes for designing chimeric antigen receptors (CARs) or small-molecule drugs, or for pharmaceutical development, achieving the growth inhibition, elimination, or enhancement of immune functions of their corresponding progenitor cells (e.g., B progenitor cells and B cells), specifically include, but are not limited to, editing, inhibiting, or activating lineage-specific genes; employing small-molecule or pharmaceutical interventions; and utilizing chimeric antigen receptors (CARs) to suppress, eliminate, or functionally enhance targeted progenitor subpopulations.

### Example 8: Application of Transcription Factors in the Reprogramming of HPC Subpopulations

a) Since Shinya Yamanaka established the induced pluripotent stem cell (iPSC) system in 2006, stem cell reprogramming via transcription factors has been widely recognized as a feasible and well-established approach in the field. By transfecting transcription factors that determine fate of HPCs, it is possible to reprogram them into lineage-specific cells, such as myeloid or lymphoid cells, that can further induce or differentiate into corresponding cell types. Currently, HSC reprogramming is favored for large-scale expansion, but genetic reprogramming carries potential tumorigenic risks. Therefore, identifying the transcription factors that determine fate of HPCs could help optimize and improve reprogramming strategies, allowing for temporal and spatial control of cell reprogramming, in combination with growth factor-induced culture, which can reduce the number of genes introduced for reprogramming and lower the risk of tumor formation.

This Example describes experimental protocols for reprogramming multipotent erythroid HPCs through overexpression and transfection of key reprogramming transcription factors. The reprogramming or intervention methods include either overexpressing one or more of the aforementioned fate-determining transcription factors and fate-determining genes in HSCs or somatic cells, or inhibiting the expression of one or more of the aforementioned fate-determining transcription factors and fate-determining genes in HSCs or somatic cells. These approaches may be implemented through one or more of the following methodologies: constructing lentiviral or adenoviral systems of overexpression, small RNA (siRNA) interference-based inhibition of transcription factor, or gRNA lentiviral or adenoviral systems of CriSPR-Cas9-based knockout systems of expression of transcription factor, small-molecule compound or pharmaceutical-mediated activation or inhibition, cell co-culture activation, growth factor/cytokine-induced activation, gene editing technologies and vector delivery systems.

### I. Transcription Factor Reprogramming Strategies

1. The cDNA sequences of GATA1 (NM_002049.4), KLF1 (NM_006563.5), and TAL1 (NM_001290403.2) (based on the transcript sequence) were inserted into the multiple cloning site of an overexpression vector (FIG. 21A), creating the multi-gene overexpression vector pLV-EF1a-GATA1-P2A-KLF1-P2A-TAL1-PGK-BSD-P2A-mCherry (FIG. 21A). The genes are linked via the P2A sequence (CGCGCCAAGCGCGGCAGCGGCGCCACCAACTTCAGCCTGCTGA AGCAGGCCGGCGACGTGGAGGAGAACCCCGGCCCC) (SEQ ID No. 4). If the number of co-expressed is large, multiple vectors can be co-transfected; The vectors conducted in this invention are multi-gene combination expression vectors, if timed and location-specific expression reprogramming is required, it can be achieved using inducible promoters (e.g., tetracycline-inducible promoters) or conducting multi-gene combination expression vectors or optogenetic techniques (light control), which are well known in the field.

2. Packaging and preparation of Lentiviral vector and corresponding virus followed Step 2 of Example 7.

3. Lentiviral transduction followed Step 3 of Example 7.

4. HEK293T cells (AST), embryonic stem (ES) cells, or human iPSC cells were transduced with lentiviruses containing the three genes (GATA1, KLF1, TAL1) to conduct a reprogrammed cell line with erythroid differentiation potential.

5. After transfection was completed, positive cells were screened using Puromycin (MCE) or Blast (HY-K1054, MCE), then transferred to suspension culture (StemSpan™ SFEM II, Catalog #09605) in vitro and induced into embryoid bodies (by adding BMP4 (HY-P7007A, MCE), bFGF (HY-P7330A, MCE), and small molecule inhibitor(Y-27632)).

6. Following approximately 48 hours of formation of an embryoid body, replacing with erythroid induction medium for reprogramming induction according to the step of erythroid induction described in Example 6 (for culturing reprogrammed cells of different lineages, supplement with lineage-specific growth factor combinations). Enhanced efficacy can be achieved by supplementing the reprogramming induction process with medium supernatant collected from umbilical cord hematopoietic stem cell cultures after 14 days of cultivation, obtained through centrifugation at 1,000×g for 20 minutes with subsequent pellet removal.

7. After 14-21 days induced differentiation culture, flow cytometry was used to assess CD235A, SLC40A1, and CD71 expression levels.

### II. Verification of Expression of Reprogramming Transcription Factor During Induction and Differentiation of Megakaryocyte and Erythroid

Cells were collected at different differentiation stages (7-day intervals) during induced differentiation in Example 6, including induced erythroid cells (erythrocyte morphology shown in FIG. 21C), megakaryocytes, and adherent cells under equivalent induction conditions as a control group (the differentiation of non-megakaryocyte or erythroid cells resulting from the induction system). Quantitative PCR (qPCR) was performed to analyze expression-level changes of reprogramming transcription factors (e.g., GATA1, KLF1, TAL1) to validate the changes of reprogramming transcription factors.

1. Megakaryocyte and erythroid induction cultures followed Example 6.

2. Cells were collected at different differentiation stages, and their RNA was extracted, reverse transcribed into cDNA, and analyzed via Q-PCR (as described in Example 4).

Under the megakaryocyte-erythroid induction culture system, qPCR results demonstrated a progressive elevation in the expression levels of reprogramming transcription factors after 7 days of induction erythroid and megakaryocyte (FIG. 21D), indicating marked activation of these transcription factors during lineage differentiation, governing cellular differentiation direction and played a key role in fate determination. Combining the expression profiles of transcription factors (FIGS. 8, and 9) with the changes of expression of transcription factors induced in vitro (FIG. 21D) in previous examples, it confirms the deterministic regulatory role of transcription factors in maturation and differentiation of erythroid. These findings further validate that these fate-determining transcription factors are functionally applicable to lineage reprogramming across diverse progenitor cell categories.

Erythroid Reprogramming Strategy and Validation of Expression of Erythroid marker CD235A by Flow Cytometry

Multi-transcription factor reprogramming technology is a well-established and widely recognized feasible technical approach within the field. Its characteristic lies in selecting one or more transcription factors from reprogramming transcription factors identified in Example 3 in the present invention, constructing them into polygenic expression vectors to establish corresponding reprogramming systems. The technical implementation and methodology of reprogramming are exemplified as follows: after lentiviral transduction with overexpression vectors of transcription factors of stem cells, significant cellular fluorescence confirms successful construction of the overexpression system (FIG. 21B). After lentiviral transfection with overexpressed transcription factors (GATA1+KLF1+TAL1), reprogrammed cells form well-defined embryoid structures under an erythroid lineage induction culture system (Example 6, FIG. 21E). Flow cytometry analysis demonstrates that CD235A expressed positively (FIG. 21F) but CD71 expressed negatively in reprogrammed cells, indicating that they have differentiation potential of partial erythroid characteristics.

Research has demonstrated that reprogramming to induced pluripotent stem (IPS) cells with megakaryocytic differentiation potential can be achieved through the three transcription factors GATA1, FLI1, and TAL1 (T. Moreau et al., 2016, Nature Communications). Similarly, reprogramming of fibroblasts to IPS cells with erythroid differentiation potential can be achieved through six transcription factors: GATA1, TAL1, LMO2, c-Myc, KLF1, and MYB (S. Capellera-Garcia et al., 2016, Cell Reports). Reprogramming to IPS cells with multi-lineages differentiation potential can be achieved through five transcription factors: ERG, GATA2, LMO2, RUNX1c, and SCL (TAL1) (K. Batta et al., 2014, Cell Reports). Reprogramming to CD34-positive (CD34⁺) IPS cells can be achieved through four transcription factors: GATA2, GFI1B, c-FOS, and ETV6 (C.-F. Pereira et al., 2013, Cell Stem Cell).

The validated and effective transcription factors for reprogramming of HPCs referenced above are the sets and combinations of transcription factors identified in the previous examples of the present invention, as specifically manifested by either: the combination of broadly expressed transcription factors in progenitor cells with lineage-specific transcription factors to achieve reprogramming toprogenitor cells with pluripotency; or the use of lineage-specific transcription factors alone to achieve reprogramming to cells of specialized types. The specific transcription factors are as follows: key transcription factors specifically expressed in progenitor cells identified in Step 3 of Example 3 in the present invention (Class I): CDK6, SOX4, SPI1, ETV6, SERPINB1, RUNX1, TSC22D1, BCL11A, FOXP1, NRIP1, IMPDH2, XBP1, ERG, LMO2, etc. Key transcription factors in megakaryocytic-erythroid progenitor factors: NFE2, LYL1, MYB, TESPA1, GATA2, KLF1, TAL1, ZBTB16, CDK4. Key transcription factors in lymphoid progenitor factors: JUN/FOS, RUNX2, HOXA9, TCF4, DDIT4, HOPX, KLF10, HOXA3, TSC22D1, etc. Other specific transcription factors of progenitor subpopulations of each stage and direction are detailed in Example 3. Six-transcription factor reprogramming system includes Class I factor LMO2 plus other four Class II factors identified in present invention; Five-transcription factor reprogramming system includes Class I factors (ERG, LMO2, RUNX1) plus Class II factor TAL1, enabling conduction of IPS with multilineage differentiation potential for the reason that this reprogramming system includes multiple Class I transcription factors.

Three-transcription factor system includes megakaryocytic-erythroid factors, enabling reprogramming to IPS cells with megakaryocytic differentiation potential. The combinatorial strategy of transcription factors with differentiation potential of erythrocytes of reprogramming has been a direction that the academic community has been exploring for a long time, with the ultimate goal of achieving erythrocytes with normal function by large-scale induction and differentiation in vitro and “artificial hematopoietic” in vitro, demonstrating exceptional application value and significance.

In summary, based on the characters of expression of transcription factor of the progenitor subpopulations in aforementioned Examples 3-4 and the hematopoietic hierarchy (Example 9), one or the combinations of multiple key transcription factors in each hematopoietic progenitor cell subpopulation and fate-determining transcription factors of each lineage identified in the present invention were proven feasible and effective when applied to reprogramming IPS of different types of hematopoietic cells. In specific implementations, coordinated overexpression of three distinct classes of transcription factors coupled with suppression of transcription factors of other lineages (e.g., LMO4 inhibition to block mast cell differentiation) forms a polygenic overexpression/suppression vector (multigene expression composition). This enables reprogramming of distinct types of progenitor cells, ultimately applicable to cell culture, transplantation, and therapeutics in vitro. Based on the fate-determining transcription factor expression profiles and the lineage hierarchy of cell subpopulations of various lineages (see Example 9), by choosing lineage-specific transcription factors, it enables reprogramming of progenitor cell subpopulations at different differentiation stages and directions. Based on the identification of characteristic transcription factors and their expression profiles of various progenitor lineages, the present invention provides a composition for inducing reprogramming of progenitor cells, specifically comprising precise selection and design of multigene expression or suppression vectors based on one or more transcription factors of distinct progenitor subpopulations, thereby achieving a reprogramming methodology for corresponding categories of progenitor cells.

The present invention does not conduct large-scale validation of the specific reprogramming effects for all transcription factors. Based on the fact that reprogramming technology is well-known in the field and is feasible, and its feasibility and effectiveness have been proven by many past examples, the genes described in the present invention. If further validation is needed, the applicant may subsequently provide corresponding experimental evidence and protocols.

### Example 9: Reconstruction of the HSC Differentiation Lineage Tree (Hematopoietic Hierarchy)

Cell subpopulations of the hematopoietic system originate from HSCs through a stepwise differentiation and maturation process. The formation of classical HSC differentiation lineage tree (Hematopoietic Hierarchy) that is well recognized in the field is a tree-like differentiation model, where HSCs initially differentiate into common lymphoid progenitors (CLPs) and common myeloid progenitors (CMPs) (FIG. 22, left part), followed by further differentiation into various progenitor cell subpopulations (K. Akashi, 2000, Nature). Although it is widely accepted that HSCs population exhibit heterogeneity and has different differentiation stages, cells are extremely rare due to the enrichment problem of HPCs in adult peripheral hematopoietic, making it extremely difficult to obtain a high proportion of HSCs and more difficult to capture hematopoietic progenitor cell subpopulations with a complete and relatively comprehensive lineage. As a result, the entire lineage of HPCs of adult peripheral hematopoietic cannot be fully captured or accurately identified; The identification and classification of HSCs into lymphoid or myeloid lineages mainly relies on in vitro culture experiments and functional assays, leading to ongoing debates regarding the definition and identification of hematopoietic progenitor cell subpopulations-especially for those at distinct differentiation stages with very low cell numbers. For a long period, progenitor cells at distinct differentiation stages do not have well-defined boundaries. Research based on new technologies reveals that progenitor cells tend to differentiate continuously rather than through stepwise stages (L. Velten et al., 2017, Nat Cell Biol.). Consequently, the accuracy and reliability of the traditional hematopoietic progenitor cell tree-model are challenged.

Compared to peripheral hematopoietic, hematopoietic in bone marrow contains a higher proportion of HSCs, and previous studies typically utilized bone marrow-derived HSCs to identify and construct HSC lineage tree. However, HPCs in bone marrow have the following limitations:

Sampling from bone marrow is invasive and difficult, limiting the sample size to only a few cases in studies (L. Velten, 2017, Nat Cell Biol), with insufficient numbers of viable cells (the number of cells of identified subpopulations that contained most cells is only a few hundred). 2.HSCs in hematopoietic in the bone marrow are usually in an undifferentiated (quiescent) state, making it difficult to capture progenitor cells at different differentiation stages from HSCs in hematopoietic in the bone marrow.

Although single-cell sequencing technology has been applied to HSC lineage detection and identification, but its identification or research methodologies exhibit the following limitations: (1) scarcity of HSCs; (2) capture and enrichment via immunomagnetic beads or flow cytometry based on surface markers of HSCs, resulting in loss of subpopulations at distinct differentiation stages; and (3) continued reliance on surface markers CD38, FLT3 (CD135), and KIT (CD117) for cell classification and identification due to insufficient cell quantities, thereby rendering definitions and categorization inherently subjective.

The present invention has innovatively solved the above-mentioned difficulties and deficiencies, achieving the following beneficial effects (FIG. 5A):

1. Overcoming the enrichment challenge of HSCs of adult peripheral hematopoietic (Example 1), with the following advantages:
a. Significantly increased enrichment ratio: the average CD34-positive rate in adult peripheral hematopoietic reaches 10% (FIG. 1).
b. Negative enrichment method ensures the integrity of subpopulations of progenitor cells at different differentiation stages.
c. Enrichment of HSCs in adult peripheral hematopoietic allow for the capture of real progenitor cell subpopulations at different differentiation stages of HSCs.
d. Combined detection of HSCs of mobilized and non-mobilized peripheral hematopoietic increased detection rates while obtaining a greater number of HSCs subpopulations at distinct differentiation stages.

2. In contrast to traditional classification and identification based on HSC surface markers CD49f, CD38, FLT3/CD135, KIT/CD117, the present invention utilized an unsupervised clustering method based on high proportion and quantity of HSCs captured in the aforementioned Example 1. Based on the data, subjective factors were excluded. More accurate and credible HSCs lineages and subpopulations were clustered and identified (Examples 2-3). Furthermore, they underwent comprehensive multidimensional validation, including expression profiles, redefinition, functional experiments, technical methodologies, and application scenarios (Examples 4-8). The results showed that part of marker genes identified across lineages overlapped with known and recognized cell lineage markers, the data of the expression profiles demonstrated high reproducibility and accuracy, and expression profile characteristics aligned with experimental expression data. These results confirmed the precision and reliability of lineage subpopulations identified by the present invention.

3. Based on the advantages of the aforementioned identified and redefined progenitor cells and reliable data of expression profiles characteristics, the present invention further clearly identified transcription factors and fate-determining genes in key HSCs lineages, with specific gene listings provided in Example 3 and Table 1. Some genes serve as known marker genes, it should be noted that the genes identified by the present invention not only included the expression information of the genes, but also showed the information of differentiation stage and differentiation direction of the gene expression in stem cells. This new characteristics with lineage characteristics and differentiation stage characteristics, such as GATA2, although known as a key myeloid transcription factor in HSCs, the present invention clearly defined the differentiation stages, expression trend and expression distribution appeared in HPCs for the first time.

In summary, given the limitations of the existing model of differentiation of HPCs and the new breakthroughs in this invention, as well as the effective identification of subpopulation lineages and differentiation stages of HSCs, especially clarifying the intermediate differentiation stages of multiple lineages, such as the intermediate differentiation stage of megakaryocyte-erythroid lineage GAP progenitor cells, this example summarized and reconstructed the HSC differentiation lineage model (hematopoietic hierarchy) based on all the data from the previous examples: the reconstructed hematopoietic hierarchy in this example (FIG. 22, right part), which is different from the traditional differentiation model (S. E. W. Jacobsen, 2019, Nat Cell Bio, FIG. 22, left part), better explained the gradual differentiation process of HSCs and progenitor cells (HSPC) lineage commitment, clearly presented the differentiation of distinct stages and directions of HSCs and upstream and downstream cell types and the fate-determining factors of progenitor cells of different lineages simultaneously, clarified the differentiation trajectories of progenitor cells of different lineages and showed the ins and outs of the formation of cells of each lineage in the hematopoietic system (FIG. 22, right part).

The differences and advantages of hematopoietic hierarchy in the present invention from classical models are specifically reflected in the following aspects:

1. The construction of the hematopoietic hierarchy was based on the data of more complete lineages and HSCs populations of different differentiation stages in adult peripheral hematopoietic: the lineage subpopulations were more complete, the effective cells of differentiation subpopulations were more, and the data were more reliable (FIGS. 1, 2, 3, 5).

2. The identification of lineage subpopulations was based on unsupervised clustering that objectively relied on the data of expression and the information of multi-gene expression; It differs from the classic model which subjectively based on the expression of only a few surface proteins of HSCs as the identification standard (FIGS. 3 and 4); this model is more credible and reliable.

3. Identification of lineage subpopulations at distinct differentiation stages and directions demonstrated high precision and reliability. it is manifested in the presence of a considerable number of known and recognized marker genes in characteristic genes (FIG. 4); at the same time, a large number of new characteristic and phased genes have been identified (FIGS. 6-11).

4. For the first time, we clarified that HPCs differentiated into three lineages at an early (priming) stage: CLPs, NMPs, and GAPs. It is generally believed that intermediate progenitor cells (Common Myeloid Progenitors, CMPs) exist and have the differentiation ability towards multiple directions during the differentiation process of traditional differentiation models. this hematopoietic hierarchy was definitively identified, fate-determining genes (e.g., GATA2) guide the emergence of branches of NMPs and GAPs during megakaryocyte-erythroid lineage differentiation (FIGS. 3, 4, 8). These genes were only expressed in the differentiation direction of GAPs, and the expression showed a trend of progressively increase. The invention revealed that CMPs did not exist, instead, they were redefined as two distinct progenitor classes that is NMPs and GAPs. Similar types were observed for signature genes such as SLC40A1 (FIGS. 8, 10). Likewise, HOPX, HOXA9, and TCF4 determined the CLP differentiation trajectory (FIGS. 9, 11).

5. For the first time, we identified two distinct intermediate-stage progenitor cells and characteristics during early lineage differentiation of megakaryocyte-erythroid progenitor cells: GAPs and MEPs. The intermediate state progenitor cells were identified; specifically, the expression of genes such as GATA1 and KLF1 only began to appear at the MEPs stage: they possess lineage characteristics and differentiation stage characteristics.

6. The Inventor identified that two branches that is the megakaryocyte-erythroid progenitor (Pro-ME) branch and the mast cell-basophil progenitor cells (MBPs) branch respectively appeared in the late stage of differentiation of megakaryocyte-erythroid progenitor cells. Genes including KIT, CDK4, and LMO4 were key factors on differentiation and fate determination of branches (FIGS. 4, 5, 8), which differs from the classic and controversial differentiation model, this hematopoietic hierarchy can clearly identify genes of lineage characteristics and differentiation stage characteristics of differentiation branches and precisely mapped the exact nodes at which branches appeared.

7. The Inventor demonstrated that the branches and pathways of differentiation mast cell-basophil progenitor cells (MBPs) and neutrophil-monocyte progenitor cells (NMPs) are totally different. Classical models propose that granulocytes are derived from the same type of HPCs. Different from both the classical and controversial differentiation models, this hematopoietic hierarchy deciphered the origins of each progenitor lineage by identifying lineage-specific and differentiation stage-specific genes of different branches. For example, GATA1 and KLF1 were always expressed throughout the differentiation path from MPC to MBP, the expression characteristics of mast cell-basophil progenitor cells were significantly different from those of neutrophil progenitor cells in NMPs. The differentiation path shows that the differentiation process of neutrophils is the most straightforward and the shortest, matching their characteristic of responding quickly to physiological and pathological needs.

8. The differentiation trajectory of lymphoid progenitor cells was consistent with that of the classical model, the main difference lied in the first-time identification of a new class of B-lineage progenitor cells: plasma progenitors.

9. The hematopoietic hierarchy described in the present invention provided a breakthrough resolution to major unresolved challenges that had persisted for over half a century in HSCs research (1961 to the present): the ambiguity surrounding differentiation trajectories during differentiation of lineages and the relationships between upstream and downstream progenitor cells. It has uniformly resolved major controversies of existing differentiation models. Furthermore, it identified lineage-specific and differentiation stage-specific transcription factors and fate-determining genes.

10. Based on the characteristics of expression profiles and differentiation trajectories of progenitor cells in the present invention, the megakaryocyte-erythroid progenitor lineages exhibited a continuous lineage formation pattern; the differentiation processes of lymphoid progenitor and monocyte progenitor cells were stepwise.

11. It was specifically noted that this hematopoietic hierarchy cannot completely resolve the identification of all differentiation lineages of all hematopoietic progenitor lineages. Specifically included, eosinophils could not definitively identify the lineage trajectory and differentiation direction. The differentiation direction and trajectory of monocyte-macrophage cells remained inadequately supported by evidence, with the possibility of originating from multiple sources. These unresolved points were indicated by dashed lines in Figures. The present invention established clearly the primary differentiation trajectories of progenitor lineages, achieved identification of most differentiation lineages of HPCs, and clarified the differentiation trajectories for the majority cells.

12. The specific hematopoietic hierarchy that the present invention newly established provided an accurate “hierarchy roadmap” for the differentiation of HSCs and HPCs, enabling various applications of signature genes and expression profiles, achieving not only the knowing of characteristics of progenitor cells and maker genes, but also the clarity of the spatiotemporal trajectory and localization of the aforementioned characteristics in differentiation of progenitor cells.

In summary, this invention first successfully enriched various lineages of HPCs, making it possible to identify and accurately redefine the progenitor cells of each lineage and to obtain lineage-specific gene expression profiles. The characteristics of lineages and gene expression further clarified interrelationships among progenitor cell subpopulations, serving as the foundation for the subsequent reconstruction of the hematopoietic hierarchy. Standalone gene expression profiles offer limited utility, the hematopoietic hierarchy constructed in the present invention achieved a qualitative leap in applications of characteristics of gene expression. The gene expression profiles corresponded to cellular lineage characteristics, as well as the hematopoietic hierarchy. Consequently, accurate regulation to cells can control them to differentiate towards desired direction or arrest at specific stages by controlling differentiation switches (fate-determining genes) at special branchpoints (where the hierarchy functions as a roadmap). Specifically, first, the cell differentiation nodes or stages were identified through the gene expression characteristics (identification), then, the fate-determining genes were regulated, finally, the effective control of differentiation path and stage of the cells was achieved, For example, inhibiting GATA1 will significantly reduce or even stop the differentiation towards megakaryocyte-erythroid lineage of cells (R. Drissen, Nat Immunol. 2016). The expression characteristics of genes enable the precise localization of the pathways and space-time for each progenitor cell subpopulation in the hematopoietic hierarchy, thereby making their application value more clear and achievable. In simple terms, knowing the control paths and clearly identifying the control switches makes it possible to achieve the control of differentiated paths. Based on the accurate identification of the fate-determining genes of each lineage by the present invention, it ultimately enables timed and targeted gene expression, as well as the spatiotemporal control of differentiation of progenitor cells.

Taking into account the advantages of the aforementioned examples and the breakthrough features of the present invention, combined with the characteristic genes of different differentiation stages and different lineages identified by the present invention, as well as the new differentiation model of HPCs lineages constructed in the present invention, the following application prospects should be recognized as protectable:

1. The transcription factors and fate-determining genes could be employed for controlling HSC differentiation trajectories, namely fate determination, which specifically includes based on spatiotemporal expression characteristics of transcription factors, control the activation or inhibition of transcription factors at specific times and locations in different differentiation stages or directions, enabling precise regulation of differentiation directions and stages of progenitor cells as exemplified in the control in differentiation directions and stages of transcription factors of the lymphoid lineage, the neutrophil-monocyte lineage, and the megakaryocyte-red hematopoietic cell lineage; which specifically also includes the reprogramming of hemopoietic stem cells by transcription factors and its applications in transplantation and treatment.

2. The fate-determining genes and characteristic genes could be utilized to identify HSC differentiation stages and determine differentiation directions of hemopoietic stem cells, which specifically manifested that the expression of characteristic genes exhibited distinct lineage specificity and stage (temporal) specificity, Therefore, the current differentiation stages and the directions which hemopoietic stem cells was about to differentiate could be identified or clarified by these genes during the process of induction or differentiation of hemopoietic stem cells, for example, the appearance or increase in the expression of genes GATA1 and SLC40A1 indicated that the differentiation direction of megakaryocyte-erythroid lineage had entered into the differentiation stage of MEPs.

3. The fate-determining genes and characteristic genes could be applied in lineage trace of HSCs differentiation, such as SLC40A1 and GATA2 which were expressed not only in early stages, but also in specific late stages of differentiation megakaryocyte-erythroid progenitor cells.

4. The fate-determining genes and characteristic genes could be utilized to regulate the differentiation stages of HSCs, maintaining cells at specific stages to preserve their differentiation pluripotency. For example, inhibition of GATA1 expression prevented progenitor cells of lymphatic system from differentiating into megakaryocyte-erythroid progenitor cells during their differentiation process. specifically, specifically, it also included characteristic genes which inhibited lineages at differentiation stages, thereby maintaining HPCs at differentiation stages without further differentiation or maturation, thus preserving their multipotency of differentiation.

5. The characteristic genes enabled selection of genes encoding membrane proteins with lineage-specific expression, enabling the sorting and enrichment of targeted progenitor cell subpopulations of different lineages, achieving high-purity enrichment of hematopoietic cell subpopulations. Consequently, subsequent monoculture and transplantation of target progenitor cells were achieved, avoiding interference from other cells, reducing contamination risks and mitigating immune reactions.

6. The gene expression characteristics enabled accurate identification and classification of progenitor cell subpopulations across lineages. Therefore, abnormalities in the quantity and state of specific progenitor cell subpopulations correlated with disease or immune status. Based on this, the genes and expression profiles described in the present invention can accurately identify and classify progenitor cells, having diagnostic value for diseases as well as application value in monitoring immunity and health status. Its principle and basis are similar to diagnosis and application of diseases and immunity in the routine hematopoietic test based on percentage and quantity of lymphocytes and granulocytes in the current clinical diagnosis process, or to application of circulating tumor cells in cancer diagnosis. In the present invention the more accurate identification and classification of progenitor cells would enable more precise applications.

7. One of the main characteristics of neoplastic hematologic disorder is abnormalities of hemopoietic stem cells, different types of hematological tumors have different abnormal states of HPCs, including the expression profile of cells and the cell state. Based on the precise classification of HPCs and expression profiles, as well as hematopoietic hierarchy, by comparing the characteristics of HPCs in hematopoietic cancer with the characteristics of HPCs identified in the present invention, the class of hematopoietic cancers could be identified, achieving the accurate classification and typing of hematopoietic tumors, which could apply to guide precision medicine and targeted therapies of different hematopoietic cancers.

8. Based on the above items 1-7 and the hematopoietic hierarchy reconstructed in this example (FIG. 22, right part), the present invention found application and study in induction, intervention, therapy, reprogramming, and transplantation of HPCs, especially application of diseases such as tumors of the hematopoietic system, aging, health management and immunity.

Special Statement: It should be specifically noted that the various applications described in the present invention were effectively enabled based on the progenitor cell lineages characteristics, marker gene characteristics, and hematopoietic hierarchy identified in the present invention. The conventional technical solutions and methods involved in these application scenarios-such as antibody design and synthesis for cell sorting, transfection protocols for genetic reprogramming, cell lineage tracing methods, and growth factor supplementation for induction culture—were well known in the field. Although there may be some differences in the details, they did not affect the ultimate scope or outcomes of practical applications.

It should be noted that since the number of genes involved in the present invention is quite large, only representative gene expression profile images and results—including expression validation, functional assays, induction culture experiments, and reprogramming experiments-were selectively presented in some examples herein. It should be understood that the methods and protocols used in the examples were universally applicable, although results might vary, equivalent validation could be performed for any other gene. The expression profiles of all genes, alongside their stage-specific and directional characteristics during progenitor cell differentiation, had been comprehensively validated through multi-angle analyses in the examples. Thus, the expression features and application value of genes not explicitly shown remained equally reliable and deserved support as evidenced by the example in various perspectives and aspects.

The original data derived from the present invention was an expression matrix, which could not be fully appended to this application or described comprehensively and accurately in word, it contains the expression profiles of all genes; The original data can be plotted and provided at any time as needed to present the expression profile results of any of the genes mentioned in this application, in order to verify reliability and authenticity, under necessary circumstances and when the third party complies with confidentiality, data could be provided for patent examination purposes. Genes that do not have the expression profile characteristics listed should not affect the scope and content of rights claimed in this application due to the absence of listed completely.

The foregoing provided a detailed description of the present invention. For those skilled in the art, it could be implemented within a broad range under equivalent parameters, concentrations, and conditions without departing from its spirit and scope. Although specific examples (involving representative genes) were provided, it should be understood that further improvements could be made to the present invention. In summary, this application intended to encompass any modifications, applications, or adaptations of the invention-including those that may extend beyond the explicitly disclosed scope but are achieved using conventional technologies known in the field.

### INDUSTRIAL APPLICABILITY

The present invention has established an efficient method for the enrichment and identification of rare hematopoietic stem and progenitor cells, obtaining highly intact HSPC populations. Based on the lineage characteristics and expression profiles characteristics of highly intact HSPC, it identified characteristic expression profiles of fate-determining genes and marker genes of progenitor subpopulations across distinct lineages, and reconstructed a progenitor differentiation lineage tree model (hematopoietic hierarchy), accurately depicting the fate-determining factors governing lineage branching and differentiation stages, lineage formation paths and patterns during the process of differentiation and formation of hematopoietic progenitor. By integrating the characteristics of progenitor cell lineages, the characteristics of marker genes and hematopoietic hierarchy identified in the present invention, the developed technical methods and protocols enabled the effective achievement of applications including, but not limited to: isolation, identification, sorting, or enrichment of hematopoietic progenitor subpopulations; quantification of hematopoietic progenitor subpopulations; preparation of high-purity progenitor cell subpopulations; inducting directed differentiation of HSCs or HPCs; controlling over HSPC differentiation direction or potential; reprogramming of HSPCs with specific or multi-lineage differentiation capacity; lineage tracing or localization; cell transplantation or cell immunotherapy; prevention or treatment of hematopoietic tumors; reversing or altering biological behavior of hematopoietic tumor cells, or inhibiting their growth; construction of in vitro drug screening models for hematopoietic tumors, or development or screening of preventive or therapeutic agents for hematopoietic tumors; resistance to or delay of aging; enhancement of immune function; preparation of hematopoietic cell or hematopoietic component products.

What is claimed is:

1. A method for identifying differentiation stages and fate-determining genes of hematopoietic progenitor cells, comprising:
(1) enriching and identifying hematopoietic progenitor cell populations in various differentiation stages and lineages;
(2) establishing gene expression profiles for each hematopoietic progenitor cell subpopulation,
(3) constructing a dynamic change and expression localization correlations between expression profile characteristics of the fate-determining gene and characteristics of the progenitor cell subpopulations; wherein the correlations comprise: the correlation between expression level, activation or inhibition state of the genes and the differentiation stages and directions, and trajectories of the progenitor cells;
(4) distinguishing lineage hierarchy characteristics comprising differentiation stages, differentiation trajectories, branches and nodes of the progenitor cell subpopulations in three different lineage directions of CLPs, GAPs and NMPs;
(5) constructing a trilineage hematopoietic hierarchy to identify differentiation stages and regulating fate-determining genes of hematopoietic progenitor cells.

2. The method of claim 1, wherein the step (1) comprises: a process of identifying the intermediate transitional progenitor cell subpopulations GAPs and NMPs and their specific marker genes.

3. The method of claim 1, wherein the method further comprises: identifying differentiation trajectories of each of the progenitor cell subpopulations, and the spatiotemporal characteristics of the fate-determining genes in the hematopoietic hierarchy.

4. The method of claim 1, wherein the step (3) comprises: identifying the activated and inhibited fate-determining genes at different differentiation stages of progenitor cells.

5. The method of claim 1, wherein the step (4) comprises: locating a lineage direction and differentiation stage of each progenitor cell subpopulation in the hematopoietic hierarchy.

6. The method of claim 1, wherein the hematopoietic progenitor cell populations comprise at least one selected from the group of: common lymphoid progenitor subpopulation (CLP), NK progenitor subpopulation (Pro-NK), T progenitor subpopulation (Pro-T), B progenitor subpopulation (Pro-B), plasma progenitor subpopulation (Pro-Plasma), neutrophil and monocyte progenitor subpopulation (NMP), megakaryocyte-erythroid lineage progenitor subpopulation (GAP), megakaryocyte-erythroid progenitor subpopulation (MEP), megakaryocyte-erythroid precursor progenitor subpopulation (Pro-ME), mast cell and basophil progenitor subpopulation (MBP), eosinophil progenitor subpopulation (Pro-Eosinophil), monocyte-macrophage progenitor subpopulation (Pro-Mac), and monocyte-dendritic cell progenitor subpopulation (Pro-DC);
wherein the common lymphoid progenitor subpopulation (CLP) expresses at least one gene selected from the group consisting of SPINK2, HOPX, HOXA9, RUNX2, LTB, IGHM, DNTT, PRSS2, SLC2A5, MME, CCR7, NKG7, LST1, BASP1, CD79A, MZB1, FLT3, and SPON1, while is negative for: CNRIP1, FCER1A, GATA1, and S100A10;
the NK progenitor subpopulation (Pro-NK) expresses at least one gene selected from the group consisting of: GNLY, NKG7, CD247, CCL5, FCGR3A, PRF1, GZMA, GZMB, KLRD1, KLRB1, KLRF1, CD3E, CD7, HOPX, IL2RB, TBX21, and ID2, while is substantially negative for IL7R and GATA3;
the T progenitor subpopulation (Pro-T) expresses at least one gene selected from the group consisting of: TCF7, IL7R, GATA3, KLRB1, CD3E, CD3D, CD7, CD247, LTB, BCL11B, and DDIT4, while is substantially negative for GNLY, FCGR3A, and GZMA;
the B progenitor subpopulation (Pro-B) expresses at least one gene selected from the group consisting of: CD19, MS4A1, FCER2, CD79A, CD79B, IGHM, LTB, IGKC, PAX5, VPREB3, CD22, CD24, and FCRLA, while is substantially negative for CD27;
the plasma progenitor subpopulation (Pro-Plasma) expresses at least one gene selected from the group consisting of: CD27, CD38, IGKC, IGHA1, SLAMF7, CD79A, CD79B, PRDM1, IRF4, JCHAIN, and IFI30, while is substantially negative for MS4A1 and FCER2;
the neutrophil and monocyte progenitor subpopulation (NMP) expresses at least one gene selected from the group consisting of: CSF3R, MPO, MGST1, IGLL1, S100A10, C1QTNF4, NPDC1, MYB, CDK4, CDCA7, CEBPA, and NPW, while is substantially negative for: GATA2, SLC40A1, CNRIP1, and LTB;
the megakaryocyte-erythroid lineage progenitor subpopulation (GAP) expresses at least one gene selected from the group consisting of: GATA2, NFE2, LYL1, MYB, SLC40A1, TESPA1, and CSF3R, while is substantially negative for: GATA1 and KLF1;
the megakaryocyte-erythroid progenitor subpopulation (MEP) expresses at least one gene selected from the group consisting of: GATA2, NFE2, LYL1, MYB, GATA1, KLF1, CSF2RB, and SLC40A1, while is substantially negative for CSF3R;
the megakaryocyte-erythroid precursor progenitor subpopulation (Pro-ME) expresses at least one gene selected from the group consisting of: HBD, CDT1, MCM2, MCM6, MCM5, MCM4, MCM3, MCM7, CDCA7, CDK4, and TYMS, while is substantially negative for: FLT3, SPINK2, HOPX, C1QTNF4, CSF3R, MS4A2 and MS4A3;
the mast cell and basophil progenitor subpopulation (MBP) expresses at least one gene selected from the group consisting of: TPSAB1, LMO4, HDC, MS4A2, TPSB2, MS4A3, KIT, PRG2, CLC, MCM2-MCM7, APOC1, MITF, and TRIB2, while is substantially negative for HBD;
the eosinophil progenitor subpopulation (Pro-Eosinophil) expresses at least one gene selected from the group consisting of: CLC, HDC, RFLNB, MEIS1, and ETV6, while is substantially negative for MS4A2, TPSB2, and MS4A3;
the monocyte-macrophage progenitor subpopulation (Pro-Mac) expresses at least one gene selected from the group consisting of: EGR1, SPI1, KLF4, CEBPB, FCGR3A, CSF1R, CD68, CD86, ITGAX, FCGR2A, LYZ, LST1, EGR2, CEBPA, MAFB, TNF, BCL6, LILRB2, CD4, CD33, FCGR2A, IFI30, S100A9, NR4A1, HMOX1, C5AR1, and CD83, while is substantially negative for: CLEC9A, THBD, and IRF8;
the monocyte-dendritic progenitor subpopulation (Pro-DC) expresses at least one gene selected from the group consisting of: CLEC9A, ANPEP, THBD, IRF8, KLF4, CD68, CD86, ITGAX, LYZ, SPI1, LST1, DDIT4, SLAMF7, BCL6, BASP1, CD4, CD33, IFI30, and CD83, while is substantially negative for: FCGR3A, CSF1R, and MAFB.

7. The method of claim 1, wherein the hematopoietic progenitor cell populations are obtained from peripheral blood and peripheral blood samples mobilized by G-CSF, and after the enrichment, CD34-positive hematopoietic progenitor cell populations reach to 10% proportion in average.

8. The method of claim 1, further comprising: identifying fate-determining genes which regulate various differentiation stages and directions of each progenitor subpopulations, and defining their spatiotemporal expression patterns and dynamic activation/inhibition features;
wherein the fate-determining genes comprise:
the fate-determining genes for the hematopoietic progenitor cell populations comprise: SOX4, CDK6, SERPINB1, FOXP1, SPI1, XBP1, ETV6, BCL11A, RUNX1, ERG, LMO2, CD82, CYTL1, EGFL7, NRIP1, IMPDH2, LY6E, ITGA4, SPINT2, EIF1, PPIA, PPIB, HMGB1, CD74, PFN1, TXN, ZFP36L2, CD37, HSP90AA1, and TMSB4X;
the fate-determining genes for the CLP subpopulation comprise: HOPX, DDIT4, HOXA9, and RUNX2;
the fate-determining genes for the Pro-NK comprise: DDIT4, HOPX, TBX21, and ID2;
the fate-determining genes for the Pro-T comprise: TCF7, GATA3, BCL11B, and DDIT4;
the fate-determining genes for the Pro-B comprise PAX5;
the fate-determining genes for the Pro-Plasma comprise: PRDM1 and IRF4;
the fate-determining genes for the NMP comprise: MYB, CDK4, and CEBPA;
the fate-determining genes for the GAP comprise: GATA2, NFE2, LYL1, and MYB;
the fate-determining genes for the MEP comprise: GATA2, NFE2, LYL1, MYB, GATA1, KLF1, ZBTB16, TAL1, CDK4, and TESPA1;
the fate-determining gene for the Pro-ME comprises CDK4;
the fate-determining genes for the MBP comprise: LMO4, CDK4, and MITF;
the fate-determining gene for the Pro-Eosinophil subpopulation comprises ETV6;
the fate-determining genes for the Pro-Mac comprise: SPI1, KLF4, CEBPB, EGR1, EGR2, CEBPA, MAFB, BCL6, and NR4A1;
the fate-determining genes for the Pro-DC comprise: SPI1, KLF4, IRF8, DDIT4, and BCL6.

9. The method of claim 1, wherein the expression profile characteristics of the fate-determining genes are configured for tracking and localizing of progenitor cells differentiation stages, so as to track and localize differentiation directions and stages of progenitor cells based on the expression profile and dynamic changes of the fate-determining genes.

10. The method of claim 1, wherein the fate-determining genes comprise: SLC40A1, CD71 and CD235A, for achieving a differentiation stage localization of erythrocyte progenitor cells during induction and differentiation process.

11. The method of claim 1, wherein the expression profile characteristics of the fate-determining genes are configured for regulation of the differentiation or function of the hematopoietic progenitor cell, wherein the function regulation comprises: regulating fate-determining genes of progenitor cell types, activating or inhibiting fate-determining genes, thereby achieving functional control over cell growth inhibition, killing, differentiation, and proliferation.

12. The method of claim 11, wherein by means of sgRNA vector inhibition of fate-determining gene HOPX, so as to inhibit the growth of NK92 tumor cell.

13. The method of claim 1, wherein the fate-determining genes comprise: a combination of progenitor cell characteristics, for temporally and spatially reprogramming hematopoietic progenitor cells with specific lineage differentiation potentials; by combining multiple transcription factors, or inhibiting transcription factors of other lineages, a multi-gene overexpression or inhibition vector is constructed to achieve the reprogramming of progenitor cells with different types of differentiation potentials, and finally used for in vitro cell culture, transplantation or treatment.

14. The method of claim 13, wherein the method comprises an operation of obtaining reprogrammed cells with erythrocyte differentiation potential by constructing three transcription factor GATA1\KLF1\TAL1 co-expression vector.

15. The method of claim 1, wherein the fate-determining gene expression profile characteristics are configured for identifying the growth factors or supplements for progenitor cell induction culture, determining the additive components, dosage and use time of the progenitor cell induction culture system, and achieving the optimization of directional induction and differentiation control of in vitro progenitor cell induction culture.

16. The method of claim 15, wherein the method comprises: increasing the differentiation proportion of erythrocytes while reducing the differentiation proportion of non-target cells by reducing a dosage of SCF factor in the late-phase culture during erythroid progenitor cell induction, based on expression characteristics of EPOR and KIT.


SEQUENCE LISTING (4 sequences)

SEQ ID NO: 1 - DNA, 20 bp, synthetic construct
  source 1..20: mol_type = other DNA
gaccgcgagcggccccacag

SEQ ID NO: 2 - DNA, 22 bp, synthetic construct
  source 1..22: mol_type = other DNA
gcggagaccgcgagcggcccca

SEQ ID NO: 3 - DNA, 23 bp, synthetic construct
  source 1..23: mol_type = other DNA
ggcggagaccgcgagcggcccca

SEQ ID NO: 4 - DNA, 78 bp, synthetic construct
  source 1..78: mol_type = other DNA
cgcgccaagcgcggcagcggcgccaccaacttcagcctgctgaagcaggccggcgacgtggaggagaaccccggcccc